SEQUENCE LISTING (12 sequences)

SEQ ID NO: 1 - protein, 8 aa, unidentified
LLESDYFR

SEQ ID NO: 2 - protein, 14 aa, unidentified
LGAVDESLSEETQK

SEQ ID NO: 3 - protein, 20 aa, unidentified
ATGTSSGANSEESTAAEFCR

SEQ ID NO: 4 - skipped

SEQ ID NO: 5 - protein, 11 aa, unidentified
EDLNYHDPTVK

SEQ ID NO: 6 - skipped

SEQ ID NO: 7 - protein, 15 aa, unidentified
DIVPGDIVEIAVGDK

SEQ ID NO: 8 - protein, 47 aa, unidentified
EDNPDFEGVDCAIFESPYPMTMALSVLVTIEMCNALNSLSENQSLLR

SEQ ID NO: 9 - protein, 7 aa, unidentified
AVLQWTK

SEQ ID NO: 10 - protein, 30 aa, unidentified
HDDSSDNFCEADDIQSPEAEYVDLLLNPER

SEQ ID NO: 11 - protein, 12 aa, unidentified
FDGILTEGEGPR

SEQ ID NO: 12 - protein, 7 aa, unidentified
NLLQNIH

(19) United States
(12) Patent Application Publication
(10) Pub. No.: US 2024/0366624 A1
(43) Pub. Date: Nov. 7, 2024

(54) METHODS AND COMPOSITIONS FOR TREATING THROMBOTIC DISEASE

(71) Applicant: Jaehyung Cho, St. Louis, MO (US)

(72) Inventor: Jaehyung Cho, St. Louis, MO (US)

(73) Assignee: Washington University, St. Louis, MO (US)

(21) Appl. No.: 18/586,471

(22) Filed: Feb. 24, 2024

Related U.S. Application Data
(60) Provisional application No. 63/486,680, filed on Feb. 24, 2023.

(51) Int. Cl. A61K 31/5415; A61K 45/00; A61P 7/02; A61P 9/10

(52) U.S. Cl. CPC A61K 31/5415; A61K 45/00; A61P 7/02; A61P 9/10

(57) ABSTRACT

Compositions and methods of treating a thrombotic disease, disorder, or condition in a subject in need thereof are provided. Exemplary compositions comprise at least one ERO1α inhibitor. Exemplary methods comprise administering to the subject at least one ERO1α inhibitor. Methods of inhibiting ERO1α in a subject having a thrombotic disease, and methods of reducing platelet thrombus formation in subject having a thrombotic disease, are also provided.

### CROSS-REFERENCE TO RELATED APPLICATIONS

This application claims priority from U.S. Provisional Application Ser. No. 63/486,680 filed 24 Feb. 2023, which is incorporated herein by reference in its entirety.

### STATEMENT REGARDING FEDERALLY SPONSORED RESEARCH OR DEVELOPMENT

This invention was made with government support under HL146559 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020389-US-NP_2024-02-24_Sequence-Listing.xml” created on 24 Feb. 2024; 10,159 bytes). The subject matter of the Sequence Listing is incorporated herein by reference in its entirety.

### FIELD

The present disclosure generally relates to methods of treating a thrombotic disease, disorder, or condition using an ERO1α inhibitor, and compositions thereof.

### BACKGROUND

Thrombotic and thromboinflammatory diseases, including atherothrombosis and ischemic stroke, are the leading cause of death in the United States. Although many antiplatelet and anti-inflammatory therapies targeting signaling molecules or receptor-ligand interactions have been used for disease treatment, these drugs increase the risk of major bleeding and impair immune responses. Thus, there is a need for alternative and improved antithromboinflammatory therapeutic methods and compositions.

### BRIEF DESCRIPTION OF THE DISCLOSURE

In one aspect of the present disclosure, a method of inhibiting ERO1α in a subject having a thrombotic disease is provided. The method comprises administering to a subject at least one ERO1α inhibitor.

In some embodiments, the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.

In some embodiments, administering the at least one ERO1α inhibitor reduces at least one of: platelet Ca2+ release, ERO1α interaction with STIM1, ERO1α interaction with SERCA2, and Cys49-Cys56 disulfide bond reoxidation in STIM1. In some embodiments, administering the at least one ERO1α inhibitor does not significantly affect at least one of: initial platelet adhesion, fibrin generation, blood loss, bleeding times, and hemostasis in the subject.

In some embodiments, the at least one ERO1α inhibitor is selected from B12, B12-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B12-22. In some embodiments, the at least one ERO1α inhibitor is B12-5. In some embodiments, the at least one ERO1α inhibitor does not comprise an anti-ERO1α antibody.

In another aspect of the present disclosure, a method of reducing platelet thrombus formation in subject having a thrombotic disease is provided. The method comprises administering to the subject at least one ERO1α inhibitor.

In some embodiments, the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.

In some embodiments, administering the at least one ERO1α inhibitor reduces at least one of: platelet Ca2+ release, ERO1α interaction with STIM1, ERO1α interaction with SERCA2, and Cys49-Cys56 disulfide bond reoxidation in STIM1. In some embodiments, administering the at least one ERO1α inhibitor does not significantly affect at least one of: initial platelet adhesion, fibrin generation, blood loss, bleeding times, and hemostasis in the subject.

In some embodiments, the at least one ERO1α inhibitor is selected from B12, B12-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B12-22. In some embodiments, the at least one ERO1α inhibitor is B12-5. In some embodiments, the at least one ERO1α inhibitor does not comprise an anti-ERO1α antibody.

In a further aspect of the present disclosure, a composition for treating a thrombotic disease in a subject in need thereof is provided. The composition comprises at least one ERO1α inhibitor.

In some embodiments, the at least one ERO1α inhibitor is selected from B12, B112-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B12-22. In some embodiments, the at least one ERO1α inhibitor is B12-5. In some embodiments, the at least one ERO1α inhibitor does not comprise an anti-ERO1α antibody. In other embodiments, the composition does not comprise an anti-ERO1α antibody. In some embodiments, the at least one ERO1α inhibitor has an IC50 value of less than about 13 μM.

In some embodiments, the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1(A-D) is an exemplary embodiment of PDI interacting with ERO1α in platelets in accordance with the present disclosure. Human platelets were treated with or without thrombin for 5 minutes and immunoprecipitated with biotinylated polyclonal anti-PDI antibodies or control IgG. After trypsin digestion, mass spectrometric analysis was conducted. FIG. 1A: Volcano plots of mass spectrometric results show proteins that bind to PDI in resting and activated platelets. FIG. 1B: Examples of the identification of the tryptic peptides LLESDYFR (SEQ ID NO: 1) and LGAVDESLSEETQK (SEQ ID NO: 2) from ERO1α in which the parent ion is observed at 521.77 and 753.37 m/z, respectively, and the parent ion mass error is 4.3 and 3.5 ppm, respectively. FIG. 1C: The peptide sequences of ERO1α that were detected after trypsin digestion. FIG. 1D: After immunoprecipitation with antibodies, the bound fraction was immunoblotted with the indicated antibodies, followed by densitometry (the band density of ERO1a/that of PDI). The data represent the mean±SD (n=3). P value determined by unpaired Student's t-test.

FIG. 2(A-K) is an exemplary embodiment of the crucial role of ERO1α (endoplasmic reticulum oxidoreductase 1a) in cremaster arteriolar and carotid arterial thrombosis without affecting tail bleeding times in mice in accordance with the present disclosure. FIG. 2 (A-C): DyLight 649-conjugated anti-CD42c and Alexa 488-conjugated rabbit IgG or polyclonal anti-ERO1α antibodies (0.3 μg/g body weight [BW]) were injected into wild type (WT) mice for intravital microscopy. Platelet accumulation and extracellular Ero1α were detected at the site of laser-induced cremaster arteriolar injury. FIG. 2A: Representative images. FIG. 2B and FIG. 2C: The median integrated fluorescence intensities of anti-CD42c (F platelet) and anti-ERO1α antibodies (F Ero1α; n=41-43 arterioles in 6 WT mice per group). FIG. 2(D-F): Intravital microscopy with WT control and Ero1α conditional knockout (CKO) and knockout (KO) mice. After laser-induced cremaster arteriolar injury, platelet accumulation and fibrin generation were detected by injection of DyLight 649-conjugated anti-CD42c and Alexa 488-conjugated anti-fibrin antibodies, respectively. FIG. 2D: Representative images. FIG. 2E and FIG. 2F: The median integrated fluorescence intensities of anti-CD42c (F platelet) and anti-fibrin (F fibrin) antibodies (n=36-44 arterioles in 6 mice per group). Quantification of the antibody signal at a different time point after laser injury. FIG. 2G and FIG. 2H: Ero1α KO mice were treated with recombinant wtERO1α or mutant ERO1α (m ERO1a) (Cys94Ser), 4 μg/g BW. After laser injury, platelets and fibrin were detected as described above (n=36-38 arterioles in 6 mice per group). FIG. 2I: The time to occlusion (TTO) was measured by a Doppler blood flow meter in WT control, Ero1α CKO/KO, and β3 KO mice after the application of 6.5% FeCl₃ to a carotid artery (n=8-11 mice per group). FIG. 2J and FIG. 2K: After amputating the tail tip (5 mm), tail bleeding times and hemoglobin (Hb) contents were measured in WT control, Ero1α CKO/KO, and β3 KO mice (n=8-9 mice per group). Bars represent the median value. P values were determined by Mann-Whitney U test (FIG. 2E, FIG. 2F, and FIG. 2(1-K)) or ANOVA and Dunn test (FIG. 2G and FIG. 2H).

FIG. 3(A-H) is an exemplary embodiment of megakaryocyte-specific or global deletion of Ero1α not affecting the expression of other proteins in accordance with the present disclosure. FIG. 3(A-E): Generation of Ero1α CKO and global KO mice. FIG. 3A: Targeting construct and homologous recombination. FIG. 3B: Southern blotting of tail genomic DNA. FIG. 3C: PCR analysis of WT and Ero1α CKO and KO mice. FIG. 3D, FIG. 3E: Immunoblotting with lysates of platelets, neutrophils, or cardiac endothelial cells (EC) (n=3 WT control and 3 CKO/KO mice). FIG. 3F, FIG. 3G, FIG. 3H: Flow cytometry was performed to measure the surface expression of αIIbβ3 integrin, glycoprotein Ibα (GPIbα), and GPVI on WT and Ero1α-null platelets treated with or without 0.025 U/ml thrombin. The geometric mean fluorescence intensity (MFI) of antibodies is shown as the mean±SD (n=3)

FIG. 4(A-G) is an exemplary embodiment of identification of novel monoclonal anti-ERO1α antibodies in accordance with the present disclosure. FIG. 4A: Electrophoresis of recombinant human ERO1α. FIG. 4B: Human ERO1α was injected into 5 mice, and antisera were tested in ELISA using immobilized ERO1α. FIG. 4C: After cell fusion, the supernatants of hybridomas were obtained and used for ELISA and isotyping. FIG. 4D: Four selected antibodies (2 μg) were stained on an SDS-PAGE gel under non-reduced and reduced conditions. FIG. 4E: The four antibodies were immunoblotted with 50 ng of wtERO1α, activity-null mutant ERO1α Cys94Ser (mERO1α), and wtPDI. FIG. 4F: Immunoblotting of anti-ERO1α antibodies (15E9) with recombinant human ERO1α and lysates of WT and Ero1α-null platelets and human platelets. FIG. 4G: The effect of novel anti-ERO1α antibodies on ERO1α activity. The data represent the mean±SD (n=3). P values determined by ANOVA and Dunnett's test. Representative gels and blots from three independent experiments.

FIG. 5(A-C) is an exemplary embodiment of ERO1α not being detected on the surface and in the releasate of activated platelets in accordance with the present disclosure. The surface amount of ERO1α was detected on mouse (FIG. 5A) and human (FIG. 5B) platelets in flow cytometry using 15E9. FIG. 5C: The releasate (Sup) and pellet of activated mouse (mplt) and human platelets (hplt) were immunoblotted with the indicated antibodies. Representative blots (n=3).

FIG. 6(A-B) is an exemplary embodiment of ERO1α molecule copy numbers in one human and mouse platelet in accordance with the present disclosure. Recombinant human ERO1α (6-80×10¹⁵ mole) and lysates of human (FIG. 6A) and mouse (FIG. 6B) platelets (10-160×10⁵ platelets) were immunoblotted with a monoclonal anti-ERO1α antibody (15E9). Using a standard curve, the number of ERO1α molecules was calculated from Avogadro's Constant (6.0×10²³ molecules/mole).

FIG. 7(A-L) is an exemplary embodiment of crucial intracellular ERO1α (endoplasmic reticulum oxidoreductase 1a) for platelet activation, aggregation, and thrombus formation in accordance with the present disclosure. FIG. 7A: Ex vivo platelet thrombus formation in a flow chamber assay. Adherent and aggregated platelets were stained with rhodamine-conjugated phalloidin and imaged by confocal microscopy. Representative images (bar, 10 μm). Surface coverage and thrombus volume were measured (mean±SD, n=4). FIG. 7(B-E): Aggregation and ATP secretion of wild type (WT) and Ero1α-null platelets were induced by 0.025 U/mL thrombin, 0.05 μg/mL CRP (collagen-related peptide), 0.5 μM A23187, or 2.5 μM ADP. Platelet aggregation (i) and ATP secretion (ii). FIG. 7F: Ero1α-null platelets were pretreated with 50 μg/mL wtERO1α or wtPDI and then with 0.025 U/mL thrombin. Quantification graphs of aggregation and ATP secretion are presented as the mean±SD (n=3-4). FIG. 7G and FIG. 7H: P-selectin exposure and αIIbβ3 integrin activation of WT and Ero1α-null platelets were analyzed by flow cytometry. The data are presented as the geometric mean fluorescence intensity (MFI) value (mean±SD, n=3-4). FIG. 7I: WT and Ero1α-null platelets were treated with or without 0.025 U/mL thrombin for 1 minute in an aggregometer. The lysates were immunoprecipitated with control IgG or anti-β3 antibodies and subjected to immunoblotting with antibodies against talin1 or β3 and densitometric analysis (mean±SD, n=3). FIG. 7J: WT and Ero1α-null platelets were treated with or without 0.025 U/mL thrombin, followed by transmission electron microscopy. The original magnification was 34 800, and the extension was 313 000. Bar, 500 nm. FIG. 7K and FIG. 7L: Intravital microscopy was performed to determine the effect of 15E9 (1-3 μg/g body weight [BW]) and a blocking anti-PDI (protein disulfide isomerase) antibody (BD34, 3 μg/g BW) on platelet thrombus formation and fibrin generation at the site of laser-induced cremaster arteriolar injury (n=27-40 arterioles in 6 mice per group). The n indicates biological replicates. P values determined by unpaired Student t test (FIG. 7(A-E) and FIG. 7(G-1)), ANOVA and Dunnett test (FIG. 7F), or Mann-Whitney U test (FIG. 7K and FIG. 7L).

FIG. 8 is an exemplary embodiment of deletion of Ero1α not inhibiting platelet aggregation induced by a high concentration of agonists in accordance with the present disclosure. WT and Ero1α-null platelets were treated with 0.1 U/ml thrombin, 0.2 μg/ml CRP, or 2 μM A23187. Platelet aggregation was measured using an aggregometer.

FIG. 9(A-C) is an exemplary embodiment of EN460 inhibiting thrombin-induced aggregation of mouse and human platelets in accordance with the present disclosure. (A) Human (FIG. 9A), WT (FIG. 9B), and Ero1α-null (FIG. 9C) platelets were pretreated with EN460, an ERO1α inhibitor, at 1, 3, or 10 μM, and treated with 0.025 U/ml thrombin to induce platelet aggregation. The data represent the mean±SD (n=3). P values determined by ANOVA and Dunnett's test (FIG. 9(A-B)) or Student's t-test (FIG. 9C).

FIG. 10(A-E) is an exemplary embodiment of 15E9 not affecting platelet activation and aggregation in accordance with the present disclosure. Mouse platelets were treated with 0.02-0.03 (FIG. 10A P-selectin, FIG. 10B aIIbβ3) or 0.025 U/ml thrombin (FIG. 10C) in the presence of 10 μg/ml mouse IgG2a (IgG2a) or a blocking anti-ERO1α antibody (15E9). Flow cytometry (FIG. 10(A-B)) and aggregation (FIG. 10C) assays were performed. FIG. 10D: Human platelets were treated with 0.025 U/ml thrombin in the presence of mouse IgG2a (mIgG2a) or 10 μg/ml 15E9 and 16B6, followed by measuring aggregation. FIG. 10E: WT and Ero1α null platelets were treated with 10 μg/ml mouse IgG1 (mIgG1) or a blocking anti-PDI antibody (BD34), followed by measuring aggregation induced by 0.025 U/ml thrombin. The data represent the mean±SD (n=3). P values determined by unpaired Student's t-test (vs. mIgG1).

FIG. 11(A-Q) is an exemplary embodiment of ERO1α (endoplasmic reticulum oxidoreductase 1α) regulating the function of Ca²⁺ signaling molecules, STIM1 (stromal interaction molecule 1) and SERCA2 (sarco/endoplasmic reticulum Ca²⁺ ATPase 2), and enhancing cytosolic Ca²⁺ levels during platelet activation in accordance with the present disclosure. FIG. 11A: Mass spectrometric analysis was performed to identify proteins interacting with ERO1α in resting and thrombin-activated human platelets. Volcano plots show proteins whose binding to ERO1α was significantly increased or decreased during platelet activation. FIG. 11B and FIG. 11C: Immunoprecipitation of ERO1α with lysates of resting and thrombin (Thr)-activated human platelets, followed by immunoblotting and densitometry. FIG. 11(D-F): Ca²⁺ release and influx of wild type (WT) and Ero1α-null platelets were assessed in response to 0.1 U/mL thrombin, 2 μM A23187, or 20 μM thapsigargin (TG), followed by the addition of 1 mM CaCl₂. The Ca²⁺ signal was quantified by the area under the curve (AUC). FIG. 11G: Immunogold electron microscopy using resting and SFLLRN (Ser-Phe-Leu-Leu-Arg-Asn, a protease-activated receptor 1 activating peptide)-activated human platelets. Ultrathin platelet sections were incubated with antibodies against PDI (protein disulfide isomerase) or ERO1α, and bound antibodies were labeled with 2 different immunogold colloids (15 nm for PDI and 10 nm for ERO1α). FIG. 11H: Bio-layer interferometry was performed using a biotinylated ERO1α biosensor. After incubation with a different concentration of wtSTIM1 and mutant STIM1 (mSTIM1), the specific interaction between ERO1α and STIM1 was measured by subtracting the nonspecific binding. The dissociation constant, K_{D}, was calculated from the Kₒₙ and K_{off} values. The data represent the mean±SD (n=3). FIG. 11(I-K): Human platelets (hplt) and WT and Ero1α-null platelets were treated with or without 0.025 U/mL thrombin for 0 to 5 minutes. After labeling the lysates with MPB, proteins were pulled down with streptavidin magnetic beads. The bound fraction was used for immunoblotting with anti-STIM1 antibodies, followed by densitometry. FIG. 11L: A schematic of mass spectrometric analysis. FIG. 11M: The representative tandem-unmodified and N-ethylmaleimide (NEM)-modified Cys49-containing STIM1 peptides, ATGTSSGANSEESTAAEFCR (988.42 m/z, SEQ ID NO: 3) and ATGTSSGANSEESTAAEFC[+125]R (1050.95 m/z, SEQ ID NO: 4), respectively. FIG. 11N: The peak areas for ATGTSSGANSEESTAAEFCR (SEQ ID NO: 3) and ATGTSSGANSEESTAAEFC[+125]R (SEQ ID NO: 4) were normalized to that of the STIM1 control peptide, EDLNYHDPTVK (SEQ ID NO: 5), which was observed at consistent levels in all samples. FIG. 11O: wtERO1α was incubated with wtSERCA2 or mutant SERCA2 (mSERCA2) (Mut). After immunoprecipitation with polyclonal anti-ERO1α antibodies, the bound fraction was blotted with anti-SERCA2 antibodies, followed by densitometry. FIG. 11P: The representative tandem NEM-modified Cys875-containing SERCA2 peptide, VSFYQLSHFLQC[+125]K (575.62 m/z, SEQ ID NO: 6). Please note that the unmodified peptide was not detected. FIG. 11Q: The peak area for the NEM-modified peptide was normalized to that of the SERCA2 control peptide, DIVPGDIVEIAVGDK (SEQ ID NO: 7), which was observed at consistent levels in all samples. The data represent the mean±SD (n=3-4 except FIG. 11N and FIG. 11Q). The n indicates biological replicates. P values determined by unpaired Student t test.

FIG. 12(A-B) is an exemplary embodiment of Ero1α deletion reducing cytosolic Ca2+ levels in agonist-stimulated platelets in the presence of Ca2+ in accordance with the present disclosure. WT and Ero1α-null platelets were treated with 0.1 U/ml thrombin (Thr)(FIG. 12A) or 2 μM A23187 (FIG. 12B) in the presence of 0.5 mM CaCl2. Ca2+ signal was quantified by the AUC. The data represent the mean±SD (n=3). P values determined by unpaired Student's t-test.

FIG. 13 is an exemplary embodiment of WT and Ero1α-null platelets equivalently labeled with calcein AM. in accordance with the present disclosure. WT control and Ero1α-null platelets are incubated with a different concentration (25-100 nM) of calcein AM, followed by flow cytometry. MFI: the mean fluorescence intensity. The data represent the mean±SD (n=3).

FIG. 14(A-C) is an exemplary embodiment of platelet PDI not regulating Ca2+ mobilization in accordance with the present disclosure. Platelets from WT control and megakaryocyte-specific PDI CKO mice were treated with 0.1 U/ml thrombin (FIG. 14A), 2 μM A23187 (FIG. 14B), or 20 μM thapsigargin (FIG. 14C), followed by the addition of 1 mM CaCl₂. The Ca2+ signal was quantified by the AUC. The data represent the mean±SD (n=3-4).

FIG. 15 is an exemplary embodiment of EN460 affecting the basal level of cytosolic Ca2+ and inhibiting Ca2+ mobilization in accordance with the present disclosure. WT mouse platelets were pretreated with vehicle (0.1% DMSO) or 5-10 μM EN460, followed by measuring Ca2+ release and influx after stimulation with 2 μM A23187 and the addition of 1 mM CaCl₂). The data represent the mean±SD (n=3). P values determined by ANOVA and Dunnett's test.

FIG. 16(A-B) is an exemplary embodiment of free thiol groups not being observed in SERCA2 during platelet activation in accordance with the present disclosure. WT mouse platelets were treated with or without 0.025 U/ml thrombin for 0-5 minutes. After labeling the lysates with MPB, proteins were pulled down with streptavidin magnetic beads. The bound fraction was blotted with anti-SERCA2 antibodies or peroxidase-conjugated streptavidin. FIG. 16A: MPB-labeled SERCA2 was not detected at 95-100-kD (arrow). FIG. 16B: MPB-labeled proteins were detected by streptavidin. The representative blots were obtained from three independent experiments.

FIG. 17(A-F) is an exemplary embodiment of identification of novel ERO1α inhibitors by high throughput screening in accordance with the present disclosure. FIG. 17A: A scheme of an ERO1α activity assay. FIG. 17B: Screening of chemical libraries in the ERO1α activity assay. FIG. 17C: The inhibitory effect of selected compounds on ERO1α activity. FIG. 17D: The inhibitory effect of selected compounds on PDI activity. FIG. 17E: The inhibitory effect of selected compounds on H₂O₂ activity. FIG. 17F: The inhibitory effect of selected compounds on thiol-reacting activity. The data represent the mean±SD (n=3). *P<0.05, **P<0.01, ***P<0.001, and ****P<0.0001 vs. vehicle control, Student's t-test.

FIG. 18(A-R) is an exemplary embodiment of novel small-molecule inhibitors recapitulating the defect in Ero1α (endoplasmic reticulum oxidoreductase 1a)-null platelets in accordance with the present disclosure. FIG. 18A: The structure of B12 and its inhibitory effect on ERO1α activity. FIG. 18(B-E): The inhibitory effect of B12 on P-selectin exposure and αIIbβ3 integrin activation in (FIG. 18B and FIG. 18C) mouse and (FIG. 18D and FIG. 18E) human platelets. FIG. 18F: The structure of B12-5 and its inhibitory effect on ERO1α activity. FIG. 18G: A docking model of B12-5-ERO1α (3AHQ) and flavin adenine dinucleotide (FAD)-ERO1α complex. There are pi-sulfur interactions of the phenothiazine head group (B112-5) or a tricyclic ring (FAD) with Met389 and Cys397. Also, there are H bond interactions between the tail/alkyl group and His255, Asn259, and Arg287. FIG. 18H: Bio-layer interferometry was performed using a biosensor conjugated with biotinylated ERO1α. After incubation with a different concentration of B12-5, the specific interaction between B12-5 and ERO1α was measured by subtracting the nonspecific binding. The K_{D} value was calculated from the Kₒₙ and K_{off} values. The represent trace (n=3). FIG. 18(I-L): The inhibitory effect of B12-5 on P-selectin exposure and αIIbβ3 integrin activation in (FIG. 18I and FIG. 18J) mouse and (FIG. 18K and FIG. 18L) human platelets. FIG. 18M and FIG. 18N: The inhibitory effect of B12-5 (10 μM) on wild type (WT) and Ero1α-null platelet aggregation induced by thrombin or CRP (collagen-related peptide). FIG. 18O through FIG. 18R: The effect of B12-5 on Ca²⁺ mobilization in (FIG. 18O and FIG. 18P) mouse and (FIG. 18Q and FIG. 18R) human platelets after stimulation with thrombin or A23187. The data represent the mean±SD (n=3-4). The n indicates biological replicates. P values determined by ANOVA and Dunnett test (FIG. 18(B-E), FIG. 18(1-L), and FIG. 18(O-R) [for B12-5]) or Student t test (FIG. 18M, FIG. 18N, and FIG. 18(O-R) [for BAPTA (1,2-bis(o-aminophenoxy)ethane-N,N,N′,N′-tetraacetic acid)]).

FIG. 19(A-Q) is an exemplary embodiment of B12-5 having an antithrombotic effect without prolonging tail bleeding times in mice in accordance with the present disclosure. FIG. 19(A-C): Ex vivo effects of B12-5. Wild type (WT; C57BL/6) mice were treated with intravenous injection of vehicle (20% polyethylene glycol [PEG]+3% dimethyl sulfoxide [DMSO]) or B12-5 (5 μg/g body weight [BW]). One hour later, blood was drawn, followed by isolating platelets. P-selectin exposure (FIG. 19A) and αIIbβ3 integrin activation (FIG. 19B) were assessed in flow cytometry. FIG. 19C: Platelet aggregation was measured after treatment with 0.025 U/mL thrombin. The data represent the mean±SD (n=4). FIG. 19D: The plasma levels of B12-5 were analyzed by liquid chromatography-tandem mass spectrometry (LC-MS/MS) after intravenous injection of the compound (5 μg/g BW) into WT mice and quantified by comparison with a standard curve of B12-5 (mean±SD, n=3). FIG. 19E and FIG. 19F: After laser-induced cremaster arteriolar injury, intravital microscopy was conducted with WT mice pretreated with intravenous injection of saline, eptifibatide (5 μg/g BW), vehicle, or B12-5 (5 μg/g BW). The median integrated fluorescence intensities of anti-CD42c (F platelet) and anti-fibrin (F fibrin) antibodies (n=37-42 arterioles in 6 mice per group). Quantification of the antibody signal at a different time point after laser injury. FIG. 19G: Comparison of B12-5 (5 μg/g BW) with eptifibatide (5 μg/g BW) and their respective controls in inhibiting FeCl₃-induced carotid artery thrombosis in WT mice. FIG. 19H and FIG. 19I: WT mice were treated with intravenous injection of vehicle control, eptifibatide (5 μg/g BW), or B12-5 (5 μg/g BW). Thirty minutes later, tail bleeding times and Hb contents were measured after amputation of the tail tip. Bars represent the median value (FIG. 19(E-1)). FIG. 19J and FIG. 19M: WT control and Ero1α conditional knockout (CKO) and knockout (KO) mice were subjected to transient middle cerebral artery occlusion (tMCAO). Twenty-three hours later, neurological deficits were assessed by the Bederson score and grip test, and infarct volume was measured as described in Methods. FIG. 19N through FIG. 19Q: WT mice were subjected to tMCAO and then treated with saline, vehicle, or B12-5 (5 μg/g BW). Twenty-three hours later, the neurological deficit and infarct volume were measured as described above. FIG. 19K and FIG. 19O: The data represent the mean±SD (n=7-8). FIG. 19L, FIG. 19M, FIG. 19P, and FIG. 19Q: The bar indicates the median with interquartile range. P values determined by unpaired Student t test (FIG. 19(A-C), FIG. 19K, and FIG. 19O) or Mann-Whitney U test (FIG. 19(E-1), FIG. 19L, FIG. 19M, FIG. 19P, and FIG. 19Q).

FIG. 20 is an exemplary embodiment of extracellular Ero1α being detected at the site of laser injury in Ero1α CKO but not KO mice in accordance with the present disclosure. Alexa 488-conjugated polyclonal anti-ERO1α antibodies (0.3 μg/g BW) were injected into WT control, Ero1α KO, and Ero1α CKO mice for intravital microscopy. The median integrated fluorescence intensities of anti-ERO1α antibodies (F Ero1α) (n=24-27 thrombi in 3 mice per group).

FIG. 21 is an exemplary embodiment of ERO1α not being detected in mouse and human plasma in accordance with the present disclosure. Recombinant human ERO1α (0.1-3 ng) and mouse and human plasma (0.01-1 μl) were immunoblotted with an anti-ERO1α antibody (15E9). The representative blots were obtained from two independent experiments. V: an empty lane.

FIG. 22 is an exemplary embodiment of ERO1α interacting with IP3Rs during platelet activation in accordance with the present disclosure. Immunoprecipitation of ERO1α with lysates of resting and activated human platelets, followed by immunoblotting with the indicated antibodies (arrows) and densitometry. The data represent the mean±SD (n=4). P values determined by ANOVA and Dunnett's test.

### DETAILED DESCRIPTION OF THE DISCLOSURE

The present disclosure is based, at least in part, on the discovery that novel small-molecule ERO1α inhibitors effectively attenuate arterial thrombus formation under thrombotic and thromboinflammatory conditions. As shown herein, platelet ERO1α plays an important regulatory role in platelet activation and aggregation (see e.g., Example 1).

The journal article “A Critical Role for ERO1α in Arterial Thrombosis and Ischemic Stroke” to Jha et al. in Circulation Research, v. 132(11), 2023, pp. e206-e222, including the associated supplemental material, is herein incorporated by reference as support of the present disclosure.

Despite advances in the understanding of the mechanisms mediating arterial thrombosis, current antiplatelet drugs blocking a signaling molecule or ligand-receptor interaction mitigate thrombotic conditions, but they often increase the risk of life-threatening bleeding. The novel EROα inhibitors described herein may be a safe, effective anti-thrombotic and anti-thromboinflammatory agent.

As described herein, small molecule ERO1α inhibitors were identified by performing a high throughput screen using a ChemDiv library. B12-5 was identified as a new ERO1α inhibitor with an IC50 value of 7.9 microM. B12-5 at 100 microM did not affect H₂O₂ and thiol activities but inhibited monoamine oxidase A (FAD coenzyme) activity by 50%. Using a wide range of cell biological studies, treatment with platelets with B112-5 at 5-10 microM significantly inhibited platelet activation and aggregation, which recapitulated the defects observed in ERO1α-null platelets. Using two mouse models of arterial thrombosis (laser-induced cremaster arteriolar thrombosis and FeCl₃-induced carotid arterial thrombosis), intravenous injection of B12-5, 5 microg/g mouse, significantly reduced platelet thrombus formation in mice. Furthermore, intravenous injection of B112-5 reduced the infarct volume in a mouse model of ischemic stroke. However, the inhibitory dose of B112-5 did not prolong bleeding times after tail amputation in mice. These results show that the novel small-molecule ERO1α inhibitor, B12-5, can effectively attenuate arterial thrombus formation under thrombotic and thromboinflammatory conditions.

ERO1α Signal Reduction, Elimination, or Inhibition by Small Molecule Inhibitors, shRNA, siRNA, or ASOs

As described herein, an ERO1α inhibitor can be used for therapy for a thrombotic disease, disorder, or condition. An ERO1α inhibitor can be used to reduce/eliminate ERO1α signals. For example, an ERO1α inhibitor can be a small molecule inhibitor of ERO1α. As another example, an ERO1α inhibitor can be a short hairpin RNA (shRNA). As another example, an ERO1α inhibitor can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g., Zhou et al. 2016 Methods Mol Biol. 1402:199-213. Except as otherwise noted herein, therefore, the process of the present disclosure can be carried out in accordance with such processes.

### ERO1A Inhibiting Agent

One aspect of the present disclosure provides for targeting of ERO1α, its receptor, or its downstream signaling. The present disclosure provides methods of treating or preventing a thrombotic disease, disorder, or condition based on the discovery that novel small-molecule ERO1α inhibitors effectively attenuate arterial thrombus formation under thrombotic and thromboinflammatory conditions.

As described herein, inhibitors or antagonists of ERO1α (e.g., antibodies, fusion proteins, small molecules) can reduce or prevent thrombosis. An ERO1α inhibiting agent can be any agent that can inhibit ERO1α activity or signaling, downregulate ERO1α protein expression, or knockdown ERO1α gene expression.

As an example, an ERO1α inhibiting agent can inhibit ERO1α signaling.

For example, the ERO1α inhibiting agent can be an anti-ERO1α antibody. As an example, the anti-ERO1α antibody can be 15E9. Furthermore, the anti-ERO1α antibody can be a murine antibody, a humanized murine antibody, or a human antibody.

As another example, the ERO1α inhibiting agent can be an anti-ERO1α receptor antibody, wherein the anti-ERO1α receptor antibody prevents binding of ERO1α to its receptor, or prevents activation of its receptor and downstream signaling.

As another example, the ERO1α inhibiting agent can be a fusion protein. For example, the fusion protein can be a decoy receptor for ERO1α. Furthermore, the fusion protein can comprise a mouse or human Fc antibody domain fused to the ectodomain of a receptor for ERO1α.

As another example, an ERO1α inhibiting agent can be B12-5, which has been shown to be a potent and specific inhibitor of ERO1α signaling.

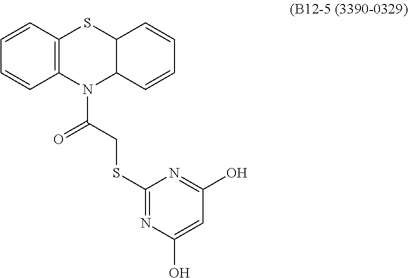 [SMILES: O=C(CSc1nc(O)cc(O)n1)N1c2ccccc2SC2C=CC=CC21]

As another example, an ERO1α inhibiting agent can be an inhibitory protein that antagonizes ERO1α. For example, the ERO1α inhibiting agent can be a viral protein.

As another example, an ERO1α inhibiting agent can be a short hairpin RNA (shRNA) or a short interfering RNA (siRNA) targeting ERO1α or an ERO1α receptor.

As another example, an ERO1α inhibiting agent can be a single guide RNA (sgRNA) targeting ERO1α or an ERO1α receptor.

Methods for preparing a ERO1α inhibiting agent (e.g., an agent capable of inhibiting ERO1α signaling) can comprise construction of a protein/Ab scaffold containing the natural ERO1α receptor as an ERO1α neutralizing agent; developing inhibitors of the ERO1α receptor “down-stream”; or developing inhibitors of ERO1α production “upstream”.

Inhibiting ERO1α can be performed by genetically modifying ERO1α in a subject or genetically modifying a subject to reduce or prevent expression of the ERO1α gene, such as through the use of CRISPR-Cas9 or analogous technologies, wherein, such modification reduces or prevents ERO1α activity or expression.

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gin by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, |
| | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, |
| | Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, ERO1α signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of ERO1α by genome editing can result in protection from a thrombotic disease, disorder, or condition.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal, addition, or modification of various thrombosis pathway signals (e.g., activation (e.g., CRISPRa), upregulation, overexpression, downregulation).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies for thrombotic disease are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies.

| | Strategy |
|---|---|
| Viral Vectors | |
| Retroviruses | Retroviruses are RNA viruses transcribing their single- |
| | stranded genome into a double-stranded DNA copy, |
| | which can integrate into host chromosome |
| Adenoviruses | Ad can transfect a variety of quiescent and proliferating |
| (Ad) | cell types from various species and can mediate |
| | robust gene expression |
| Adeno- | Recombinant AAV vectors contain no viral DNA and can |
| associated | carry ~4.7 kb of foreign transgenic material. They |
| Viruses | are replication defective and can replicate only while |
| (AAV) | coinfecting with a helper virus |
| Non-viral | |
| vectors | |
| plasmid DNA | pDNA has many desired characteristics as a gene |
| (pDNA) | therapy vector; there are no limits on the size or |
| | genetic constitution of DNA, it is relatively inexpensive |
| | to supply, and unlike viruses, antibodies are not |
| | generated against DNA in normal individuals |
| RNAi | RNAi is a powerful tool for gene specific silencing that |
| | could be useful as an enzyme reduction therapy or |
| | means to promote read-through of a premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a thrombotic disease, disorder, or condition in a subject in need of administration of a therapeutically effective amount of an ERO1α inhibitor, so as to reduce platelet accumulation, platelet activation, arterial thrombogenesis, thrombus formation, neurologic deficits, or infarct volume, or increase time to occlusion (TTO).

Methods described herein are generally performed on a subject in need thereof. A subject in need of the therapeutic methods described herein can be a subject having, diagnosed with, suspected of having, or at risk for developing thrombotic disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an ERO1α inhibitor is, for example, an amount that would cause the desired therapeutic effect in a subject while minimizing undesired side effects. In various embodiments, an effective amount of an ERO1α inhibitor described herein can substantially reduce platelet accumulation, platelet activation, arterial thrombogenesis, thrombus formation, neurologic deficits, or infarct volume, or increase time to occlusion (TTO).

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of ERO1α inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce platelet accumulation, platelet activation, arterial thrombogenesis, thrombus formation, neurologic deficits, or infarct volume, or increase time to occlusion (TTO).

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an ERO1α inhibitor can occur as a single event or over a time course of treatment. For example, an ERO1α inhibitor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for a thrombotic disease, disorder, or condition.

An ERO1α inhibitor can be administered simultaneously or sequentially with another agent, such as an antibiotic, an anti-inflammatory, or another agent. For example, an ERO1α inhibitor can be administered simultaneously with another agent, such as an antibiotic or an anti-inflammatory. Simultaneous administration can occur through administration of separate compositions, each containing one or more of an ERO1α inhibitor, an antibiotic, an anti-inflammatory, or another agent. Simultaneous administration can occur through administration of one composition containing two or more of an ERO1α inhibitor, an antibiotic, an anti-inflammatory, or another agent. An ERO1α inhibitor can be administered sequentially with an antibiotic, an anti-inflammatory, or another agent. For example, an ERO1α inhibitor can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the ERO1α inhibitor may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, an ERO1α inhibitor such as a compound B12-5 may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to ERO1α inhibitors. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: A Critical Role for Ero1a in Arterial Thrombosis and Ischemic Stroke

This example describes identification of the critical role of platelet ERO1α as a novel Ca²⁺ regulator that enhances cytosolic Ca²⁺ levels under thrombotic conditions, including arterial thrombosis and ischemic stroke.

### Introduction

Platelet adhesion and aggregation play a crucial role in arterial thrombosis and ischemic stroke. Although current anti-platelet therapies reduce thrombogenesis, the risk of hemorrhage remains substantial. It is known that platelet surface ERO1α (endoplasmic reticulum oxidoreductase 1a) influences platelet function by regulating the activity of PDI (protein disulfide isomerase). Further, protein activity is generally known to be regulated by the modification of allosteric disulfide bonds.

Herein is identified platelet ERO1α (endoplasmic reticulum oxidoreductase 1a) as a novel regulator of Ca²⁺ signaling and a therapeutic, pharmacological target for treating thrombotic diseases.

Intravital microscopy, animal disease models, and a wide range of cell biological studies were utilized to demonstrate the pathophysiological role of ERO1α in arteriolar and arterial thrombosis and to prove the importance of platelet ERO1α in platelet activation and aggregation. Mass spectrometry, electron microscopy, and biochemical studies were employed to elucidate the molecular mechanism. Novel blocking antibodies and small molecule inhibitors were used to investigate whether targeting intracellular ERO1α ERO1α can be targeted to attenuate thrombotic conditions.

Megakaryocyte-specific or global deletion of Ero1α in mice similarly reduced platelet thrombus formation in arteriolar and arterial thrombosis without affecting tail bleeding times and blood loss following vascular injury. Platelet ERO1α localization was observed exclusively in the dense tubular system and promoted Ca²⁺ mobilization, platelet activation, and aggregation. Platelet ERO1α directly interacted with STIM1 (stromal interaction molecule 1) and SERCA2 (sarco/endoplasmic reticulum Ca²⁺-ATPase 2) and regulated their functions. Such interactions were impaired in mutant STIM1-Cys49/56Ser and mutant SERCA2-Cys875/887Ser.

ERO1α modified an allosteric Cys49-Cys56 disulfide bond in STIM1 and a Cys875-Cys887 disulfide bond in SERCA2, contributing to Ca²⁺ store content and increasing cytosolic Ca²⁺ levels during platelet activation. Inhibition of Ero1α with small-molecule inhibitors but not blocking antibodies attenuated arteriolar and arterial thrombosis and reduced infarct volume following focal brain ischemia in mice.

These results demonstrate that ERO1α acts as a thiol oxidase for Ca²⁺ signaling molecules, STIM1 and SERCA2, and enhances cytosolic Ca²⁺ levels, promoting platelet activation and aggregation. This study provides evidence that targeting ERO1α can effectively attenuate thrombotic events.

As disclosed herein, deletion or inhibition of Ero1α in mice reduces platelet thrombus formation in arteriolar/arterial thrombosis and infarct volume in experimental ischemic stroke without increasing tail bleeding times and blood loss. Platelet ERO1α localizes exclusively in the dense tubular system and modifies an allosteric disulfide bond in STIM1 (stromal interaction molecule 1) and SERCA2 (sarco/endoplasmic reticulum Ca²⁺ ATPase 2), regulating cytosolic Ca²⁺ levels and platelet activation. Consequently, targeting ERO1α is a strategy to attenuate the pathogenesis of thrombotic diseases.

The present disclosure reveals novel functions of platelet ERO1α in regulating Ca²⁺ signaling and platelet activation during arterial thrombosis and ischemic stroke. Unlike previous reports, it was found that platelet ERO1α is not released or detected on the platelet surface, but it is localized in the organelle where Ca²⁺ is stored. Upon platelet activation, ERO1α interacts with Ca²⁺ signaling molecules and regulates cytosolic Ca²⁺ levels. As demonstrated herein using mouse studies with a novel ERO1α inhibitor identified by a high throughput screen, inhibition of Ero1α effectively attenuates arterial thrombosis and tissue damage in ischemic stroke without prolonging tail bleeding times. Arterial thrombotic diseases, including coronary artery disease and ischemic stroke, result in >30% of all deaths globally. Underlying these pathologies is increased platelet activity. After arterial injury, platelets adhere to collagen and von Willebrand factor (VWF) through GP (glycoprotein) VI and the GP Ib-IX-V complex, respectively, and aggregate through the interaction between fibrinogen and activated αIIbβ3 integrin, resulting in vaso-occlusive thrombosis. A similar mechanism also occurs to stop bleeding after tissue injury. Therefore, platelets are essential for thrombosis and hemostasis. Although the diverse ligand-receptor interactions trigger different signaling pathways during platelet activation, they all increase cytosolic Ca²⁺ levels. Like other cell types, platelet STIM1 (stromal interaction molecule 1) has an essential role in detecting Ca²⁺ depletion in the dense tubular system (DTS) and activating store-operated Ca²⁺ entry (SOCE), contributing to arterial thrombosis and hemostasis. Reuptake of Ca²⁺ into the DTS is mediated by SERCAs (sarco/endoplasmic reticulum Ca²⁺ ATPases), and STIM1 also contributes to SERCA-mediated Ca²⁺ store refilling in activated platelets. Current antiplatelet therapies blocking a signaling molecule or a ligand-receptor interaction reduce the morbidity and mortality associated with thrombotic disease, but they increase the risk of major bleeding. Therefore, many efforts have been made to identify a novel therapeutic target for a safer antiplatelet agent.

It has been previously demonstrated that targeting extracellular PDI (protein disulfide isomerase) might be a therapeutic strategy for treating thrombotic disease. However, treatment with blocking anti-PDI antibodies prolongs tail bleeding times in mice, raising a concern that selective PDI inhibitors may perturb hemostatic function. Moreover, cell-permeable PDI inhibitors may cause serious side effects due to the indispensable role of PDI in protein folding in the endoplasmic reticulum and cell viability. ERO1 (endoplasmic reticulum oxidoreductase 1) oxidizes PDI via a disulfide bond exchange during oxidative protein folding. Of the two isoforms found in mammalian cells, ERO1α is ubiquitously expressed, whereas ERO1β is predominantly found in intestinal and pancreatic β-cells. Unlike mice lacking Pdi, which display embryonic lethality, mice with loss-of-function mutations of Ero1-I and Ero1-Iβ are viable with a moderate defect in secretory protein production, implicating the role of ERO1 in disulfide bond oxidation and the presence of an ERO1-independent mechanism of protein folding. A recent study shows that extracellular ERO1α-PDI forms an electron transport system, regulating platelet function. However, the authors used concentrations of ERO1α and PDI (0.1-0.5 μM), which are much greater than those detected under healthy and disease conditions (around 10-100 μM), which makes the finding physiologically inapplicable. Confocal microscopy and biochemical studies suggest that ERO1α colocalizes with PDI and αIIbβ3 on the surface of unstimulated platelets and that treatment with polyclonal anti-ERO1α antibodies abrogates platelet aggregation. However, this study did not show how intracellular ERO1α is released from platelets. More importantly, none of the studies demonstrate the contribution of ERO1α to the pathology of thrombogenesis.

Using megakaryocyte-specific Ero1α conditional knockout (CKO) and global knockout (KO) mice, novel blocking antibodies, and small-molecule inhibitors, it is herein demonstrated that platelet Ero1α has a crucial role in platelet thrombus formation at the site of cremaster arteriolar and carotid arterial injuries. Immunogold electron microscopy and biochemical studies reveal that ERO1α is not released from platelets and detected on the platelet surface but localizes exclusively in the DTS. Intriguingly, ERO1α contributes to Ca²⁺ store content and promotes Ca²⁺ mobilization in a PDI-independent manner, resulting in platelet activation and aggregation. STIM1 and SERCA2 were identified as ERO1α binding partners in platelets. ERO1α interacts with the N-terminal endoplasmic reticulum luminal domain of STIM1 during platelet activation. In contrast, binding of ERO1α to SERCA2 is decreased upon agonist stimulation. Importantly, ERO1α alters a Cys49-Cys56 disulfide bond in STIM1 and a Cys875-Cys887 disulfide bond in SERCA2, regulating their functions. Treatment of mice with a novel small-molecule ERO1α inhibitor attenuates arterial thrombogenesis and infarct volume in ischemic stroke without increasing tail bleeding times. The results of the present disclosure provide evidence that targeting ERO1α may be an effective therapeutic strategy for treating thrombotic disease.

### Methods

### Mice

Wild type (WT; C57BL/6J), P3 KO, and BALB/cJ mice were obtained from The Jackson Laboratory (Bar Harbor, ME). WT control (P4hb^{flox/flox; Pf4-cre-/-}) and megakaryocyte-specific Pdi CKO mice (P4hb^{flox/flox; Pf4-cre+/-}) were generated by crossing Pf4-Cre mice with P4hb^{flox/flox} mice. Ero1α CKO and KO mice were generated as described herein elsewhere. All animal care and experimental procedures were approved by the Institutional Animal Care and Use Committee at the Washington University School of Medicine. Animals were assigned randomly to the different experimental groups.

### Isolation of Platelets, Neutrophils, and Cardiac Endothelial Cells

Human platelets, mouse platelets, neutrophils, and cardiac endothelial cells were isolated and prepared as described previously.

### Intravital Microscopy

Intravital microscopy was performed in a mouse model of laser-induced cremaster arteriolar injury as previously described. WT (C57BL/6), littermate WT control, and CKO and KO male mice (8-10 weeks old) were anesthetized with intraperitoneal injection of ketamine and xylazine. The cremaster muscle was exteriorized and superfused with 37° C. bicarbonate-buffered saline throughout the experiment. Arteriolar wall injury was induced by laser ablation (Photonics Instruments) as previously described. Multiple thrombi were generated in at least 3 to 4 different arterioles of 1 mouse, with new thrombi formed upstream of earlier thrombi to minimize any contribution from thrombi generated earlier in the mouse. Platelets and fibrin were visualized by infusion of DyLight 649-conjugated anti-CD42c (0.2 μg/g body weight [BW]) and Alexa Fluor 488-conjugated anti-fibrin (0.2 μg/g BW) antibodies, respectively. To detect extracellular Ero1α, Alexa Fluor 488-conjugated rabbit IgG or nonblocking anti-ERO1α antibodies (0.3 μg/g BW) were injected into WT (C57BL/6), WT control, Ero1α KO, and Ero1α CKO mice. In some experiments, wtERO1α or activity-null ERO1α-Cys94Ser (mutant ERO1α [mERO1α]; 4 μg/g BW) was infused into Ero1α KO mice before imaging. To test the effect of ERO1α inhibitors or blocking anti-ERO1α antibodies, WT (C57BL/6) mice were treated with intravenous injection of vehicle control, eptifibatide (5 μg/g BW), B12-5 (5 μg/g BW), isotype control IgGs, anti-ERO1α (15E9, 1-3 μg/g BW), or anti-PDI (BD34, 3 μg/g BW) 3 minutes before treatment with the anti-CD42c and anti-fibrin antibodies. The same dose of eptifibatide was injected every 20 minutes to maintain the inhibitory effect. The experiments were performed in a single-blind fashion in which the investigators did not know the identity of the sample or mouse. Fluorescence and bright-field images were captured in 6 to 10 different cremaster arterioles with a diameter of 30 to 45 μm in each mouse and recorded using either an Olympus BX61W microscope with a 60×1.0 salt water immersion objective or a Zeiss Axio Examiner Z1 microscope system with a Yokogawa confocal spinning disk (CSU-W1) equipped with 4 stack laser system (405-, 488-, 561-, and 637-nm wavelengths). Images were collected with a high-speed, high-resolution camera (2304×2304 pixel format; ORCA-Fusion BT sCMOS, Hamamatsu). Time 0 was set to when image capture began on each vessel. The data were analyzed using Slidebook (version 6.0; Intelligent Imaging Innovations). Due to the significant variation between vessels in the same mouse, the result from each vessel was counted as an individual value. Representative images were chosen that most closely resemble the mean values in the quantifications.

### Ca²⁺ Mobilization

Ca²⁺ mobilization was measured as described previously. WT control and Ero1α-null platelets (1×10⁸/mL) were suspended in HEPES-Tyrode buffer, pH 7.4. Cells were incubated with a Ca²⁺ dye (FLIPR Ca²⁺5 assay kit; Molecular Devices) for 30 minutes at 37° C. in the dark, followed by stimulation with thrombin (0.1 U/mL), A23187 (2 μM) or thapsigargin (10-20 μM). After measuring Ca²⁺ release, 1 mM CaCl₂) was immediately added to assess Ca²⁺ influx. In other experiments, Ca²⁺ dye-labeled human and WT mouse platelets were incubated with a vehicle or an inhibitor for 10 minutes at 37° C. Cytosolic Ca²⁺ levels were measured using a FlexStation3 microplate reader with an excitation wavelength of 485 nm and an emission wavelength of 525 nm. Ca²⁺ mobilization was quantified by the area under the curve and expressed as a relative fluorescence unit.

### Statistical Analysis

Detailed statistical methods were specified in the figure legends. For comparisons of two groups, unpaired 2-tailed Student t test or Mann-Whitney U test (for non-normally distributed data) was used. For 1-way parametric tests, 1-way ANOVA with Dunnett or Tukey multiple comparison test were used. For 1-way nonparametric tests, Kruskal-Wallis test with Dunn multiple comparison tests was used. GraphPad Prism 9 (version 9.5.1) was used for statistical analysis.

### Results

Ero1α has a Crucial Role in Platelet Thrombus Formation after Cremaster Arteriolar and Carotid Arterial Injuries but Does Not Affect Tail Bleeding Times in Mice

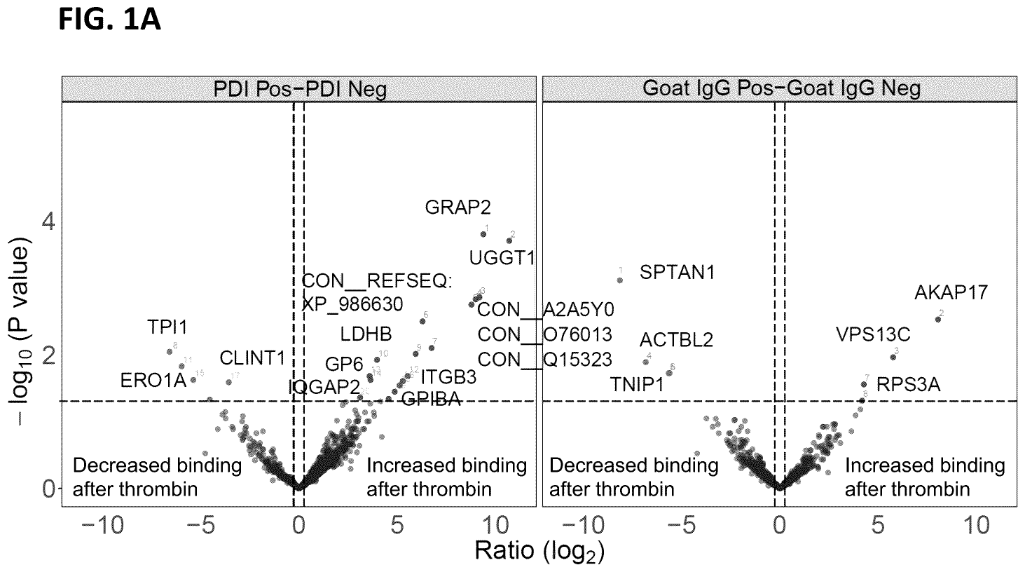

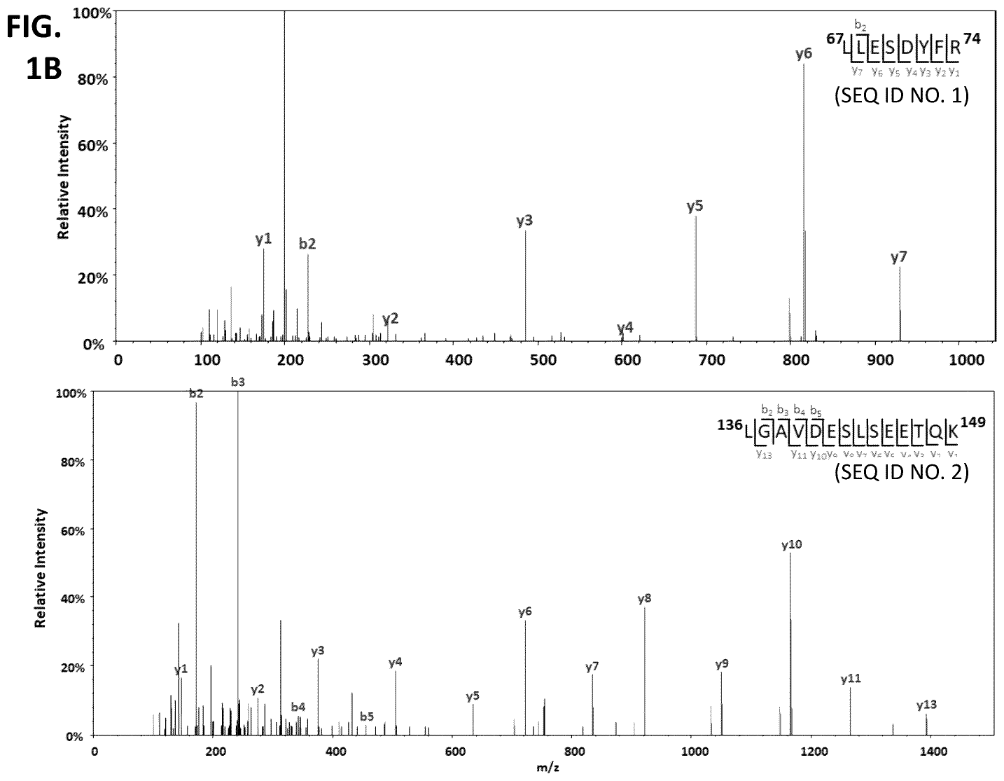

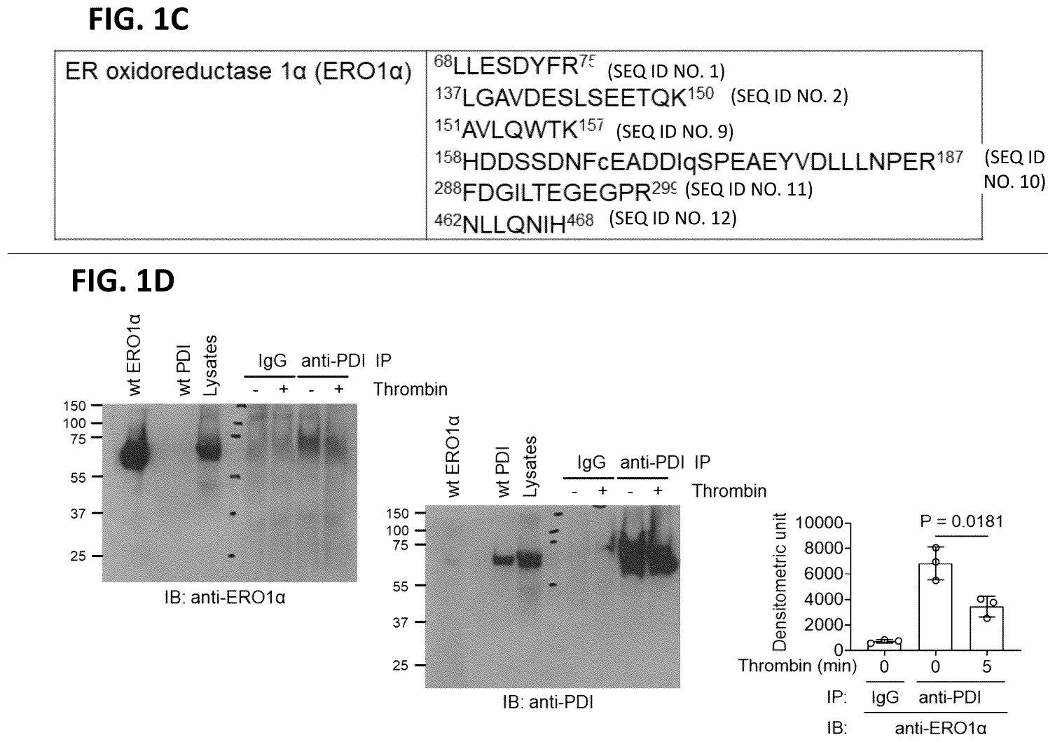

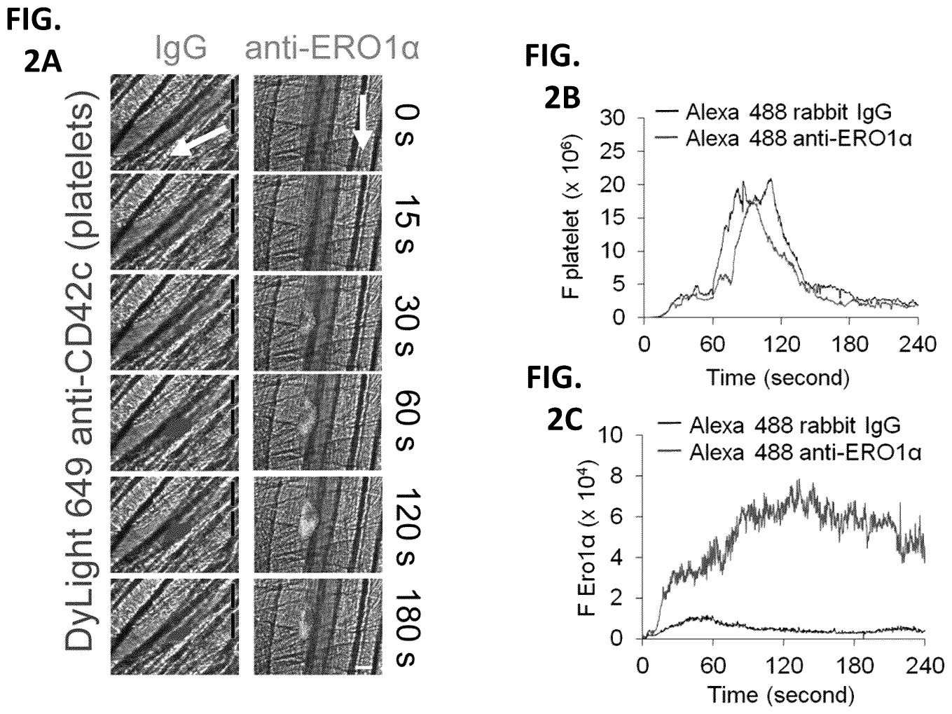

Platelet PDI has an important role in arterial thrombosis and ischemic stroke. However, it is unclear how platelet PDI activity is regulated in thrombosis. Thus, the present disclosure sought to identify molecules that interact with PDI and may regulate its activity during platelet activation. PDI was immunoprecipitated in lysates of resting and thrombin-activated human platelets and conducted mass spectrometry. PDI interacted with many intracellular and surface molecules, including uridine diphosphate-glucose GP glucosyltransferase 1, serpins, ERO1α, β3 integrin, and GP Ibα (FIG. 1A). Among them, ERO1α drew interest (FIG. 1B and FIG. 1C) as it is a known endoplasmic reticulum oxidase of PDI. It was verified that PDI interacted with ERO1α, and the interaction was decreased during platelet activation (FIG. 1D). FIG. 1C lists LLESDYFR (SEQ ID NO: 1), LGAVDESLSEETQK (SEQ ID NO: 2), AVLQWTK (SEQ ID NO: 9), HDDSSDNFcEADDIqSPEAEYVDLLLNPER (SEQ ID NO: 10), FDGILTEGEGPR (SEQ ID NO: 11), and NLLQNIH (SEQ ID NO: 12).

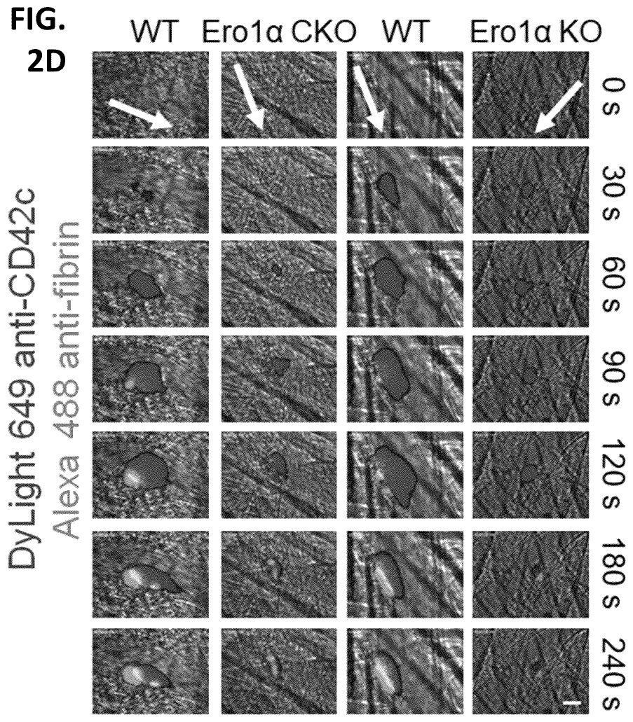

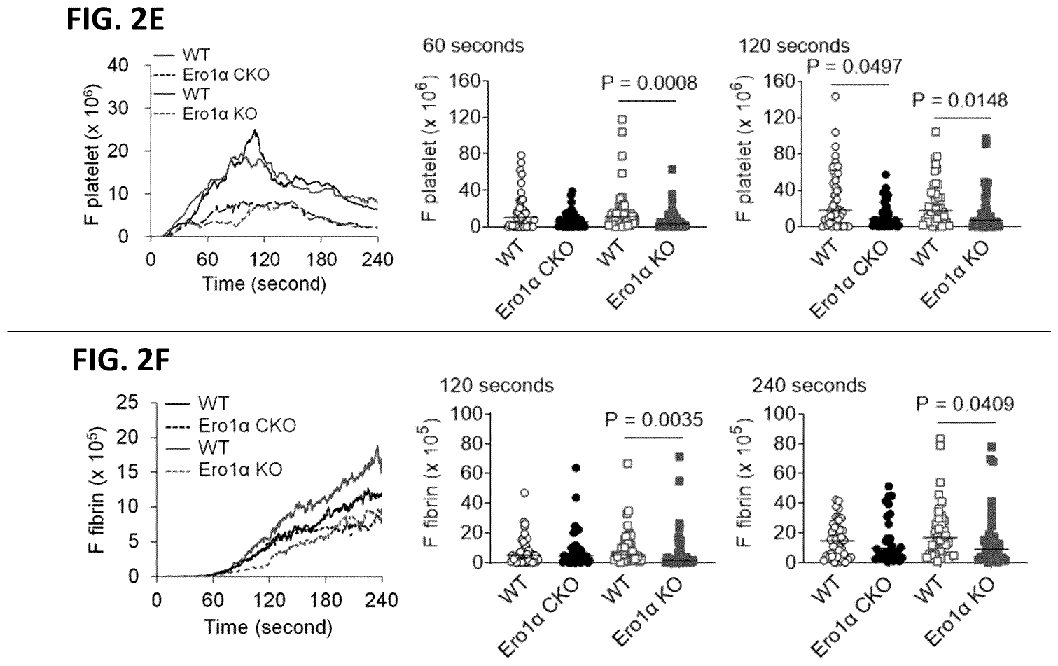

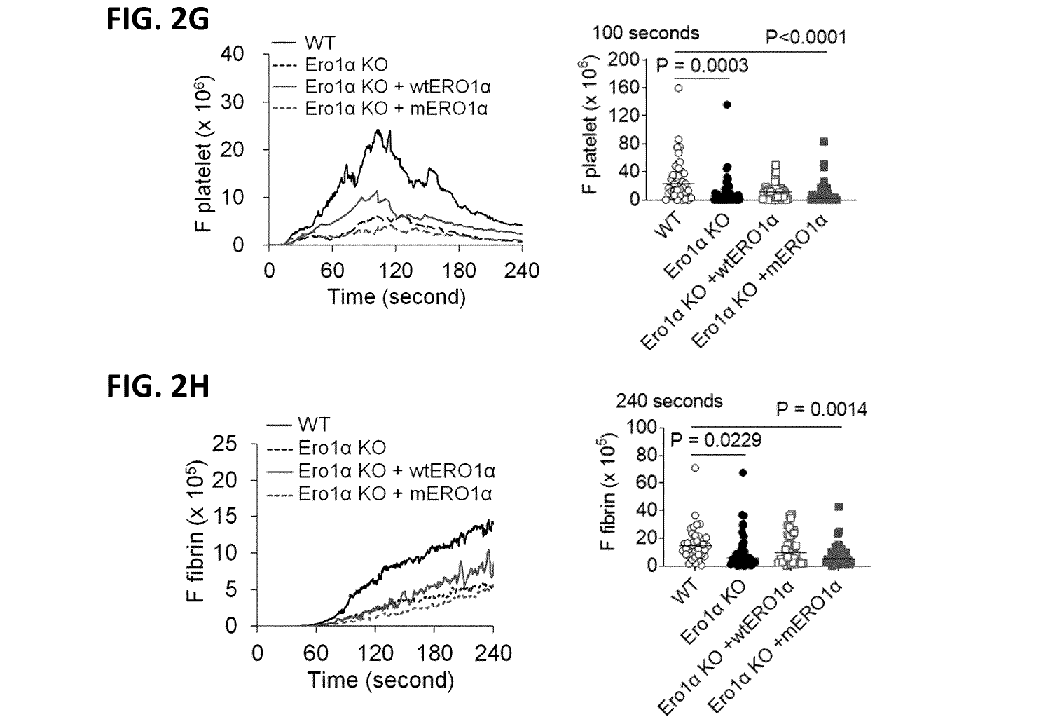

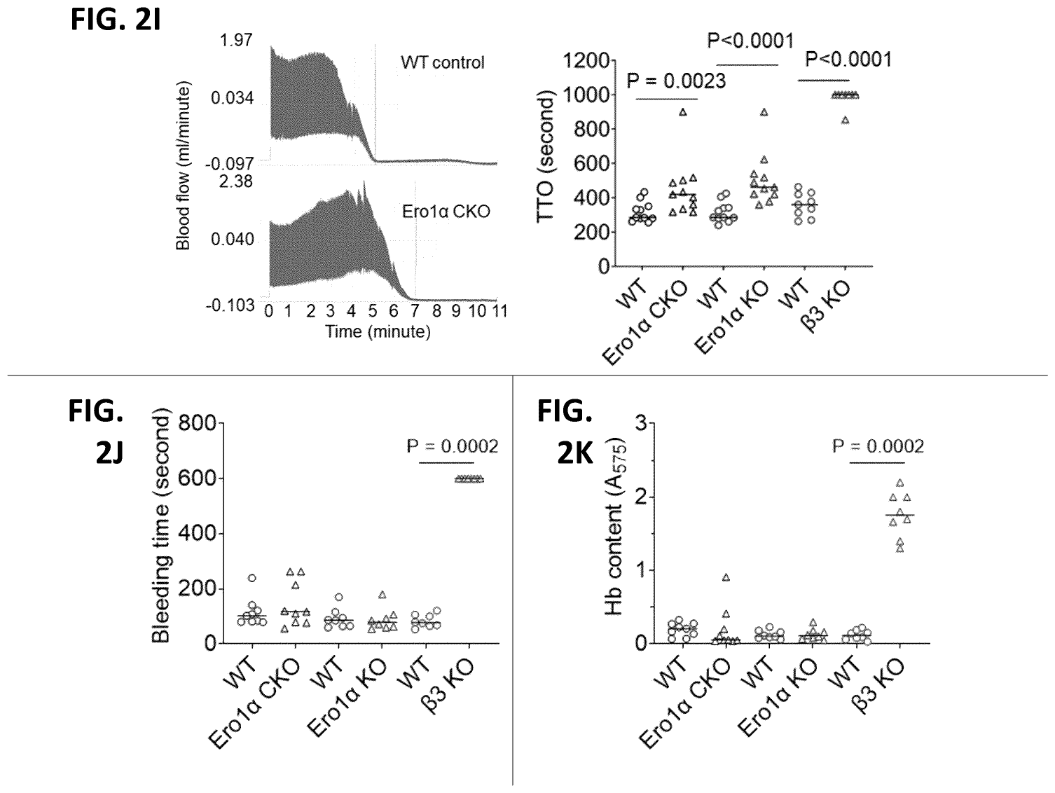

Since ERO1α has been reported to colocalize with PDI and αIIbβ3 integrin on the platelet surface, intravital microscopy was performed to detect extracellular Ero1α at the site of laser-induced cremaster arteriolar injury in mice. Using fluorescently labeled nonblocking polyclonal anti-ERO1α antibodies, Ero1α was found to accumulate at the site of laser-induced cremaster arteriolar injury (FIG. 2A through FIG. 2C). However, unlike PDI whose accumulation is significantly reduced as the size of platelet thrombi is decreased, the signal of extracellular Ero1α remained unaltered 4 minutes after laser injury. This result suggests that platelets are unlikely to be the main source of extracellular Ero1α.

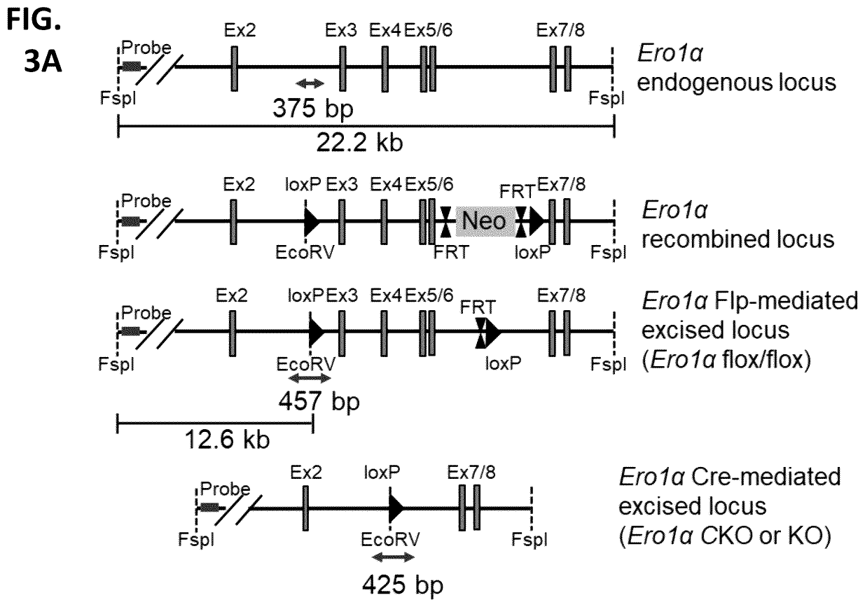

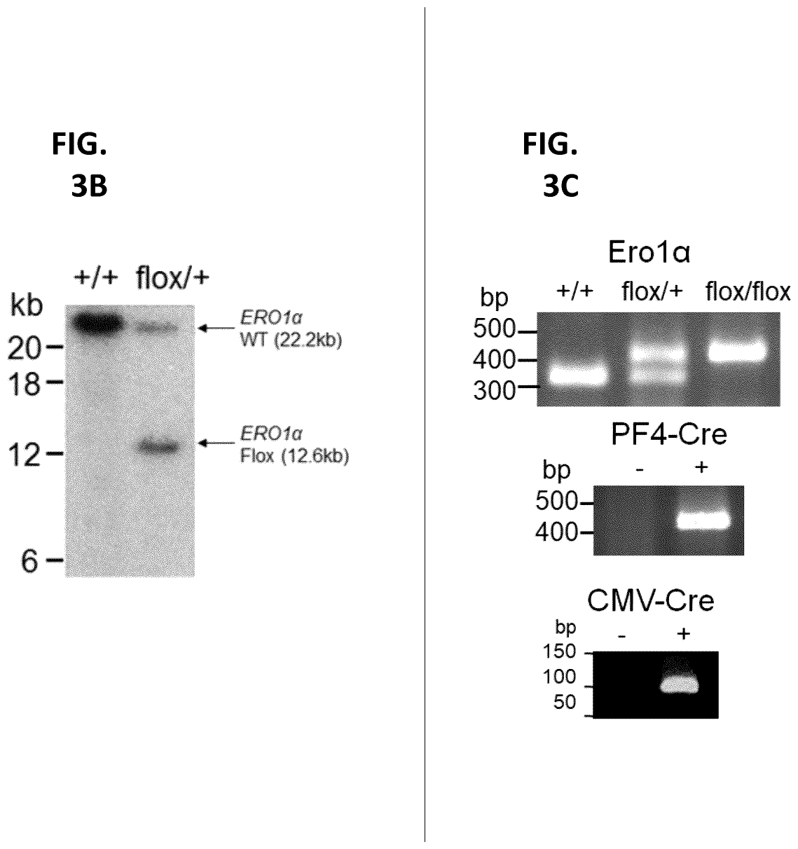

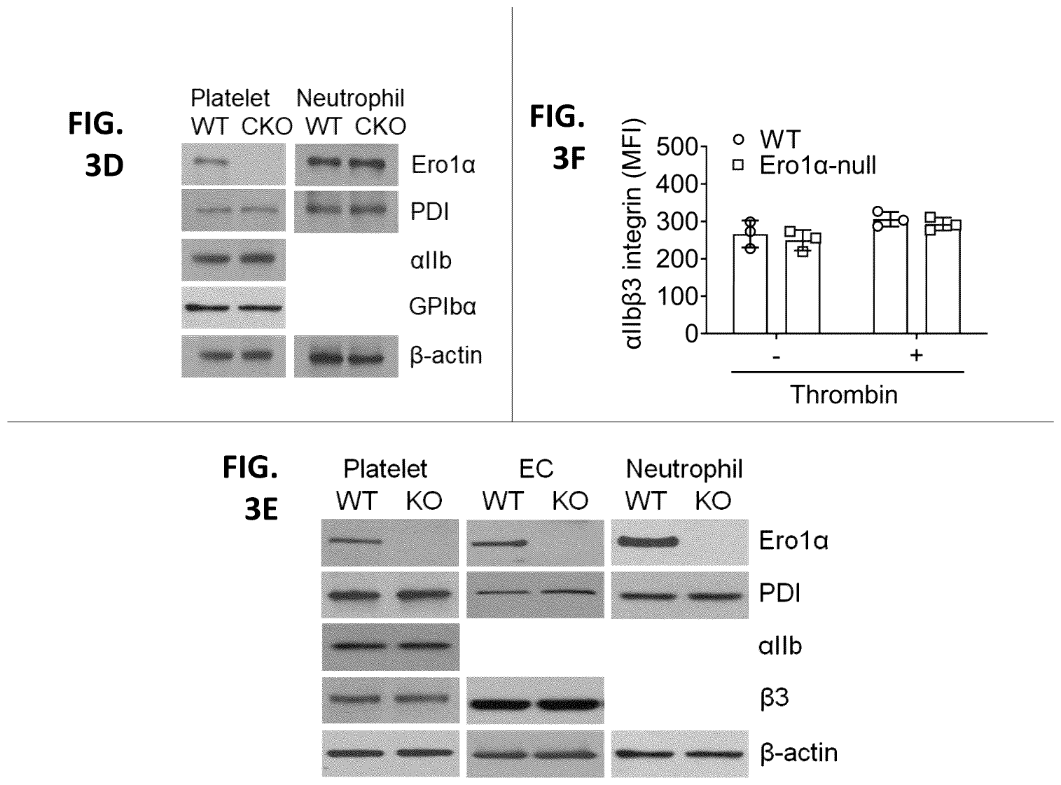

To investigate the role of ERO1α in arterial thrombosis, Ero1-I^{fl/fl} mice were generated and bred with Pf4-cre and Cmv-cre mice to delete Ero1α in megakaryocytes (Ero1α^{fl/fl;Pf4-cre+/-}) and all tissues (Ero1α^{-/-;Cmv-cre+/-}), respectively (FIG. 3A through FIG. 3C). Ero1α KO mice were viable, fertile, and born in a normal Mendelian ratio without any abnormality, suggesting that Ero1α is dispensable for embryonic development. Megakaryocyte-specific and global deletion of Ero1α did not alter the expression of other proteins, such as Pdi, αIIbβ3 integrin, GP Ibα, and GP VI (FIG. 3D through FIG. 3H), and did not affect complete blood counts (Tables 1 and 2). Intravital microscopy using the CKO and KO mice revealed that either megakaryocyte-specific or global deletion of Ero1α significantly mitigated platelet accumulation after laser-induced cremaster arteriolar injury without inhibiting initial platelet adhesion (FIG. 2D through FIG. 2F). Fibrin generation was reduced only in Ero1α KO mice. These results indicate that platelet Ero1α mainly contributes to platelet thrombus formation, whereas other cellular Ero1α plays a role in fibrin generation. The defect in platelet thrombus formation in megakaryocyte-specific Pdi CKO mice was completely rescued by injection of recombinant wtPDI but not its activity-null mutant. However, injection of recombinant wtERO1α at 4 μg/g body weight (BW) into Ero1α KO mice exhibited a minimal rescue effect on platelet thrombus formation and fibrin generation (FIG. 2G and FIG. 2H). Its activity-null mERO1α did not show any effect. This indicates that extracellular Ero1α minimally contributes to thrombogenesis.

TABLE 1
Complete Blood Counts in WT Littermate Control and Ero1α CKO Mice.
Blood cells from mice were counted using HEMAVET 950 (Drew Scientific).

| | WBC, | NE, | LY, | MO, | RBC, | PLT, | |
|---|---|---|---|---|---|---|---|
| | 10³/μL | 10³/μL | 10³/μL | 10³/μL | 10⁶/μL | 10³/μL | MPV, fL |
| WT | 7.1 ± 1.9 | 1.4 ± 0.3 | 4.2 ± 0.8 | 0.2 ± 0.1 | 10.1 ± 0.6 | 1208 ± 171 | 4.9 ± 0.6 |
| Ero1α | 6.5 ± 1.4 | 1.1 ± 0.3 | 4.3 ± 0.6 | 0.1 ± 0.0 | 10.3 ± 0.9 | 1128 ± 123 | 4.4 ± 0.3 |
| CKO | | | | | | | |

The data represent the mean ± SD (n = 8-10 mice [8-12 wk old] per group). CKO indicates conditional knockout; Ero1α, endoplasmic reticulum oxidoreductase 1α; LY, lymphocyte; MO, monocyte; MPV, mean platelet volume; NE, neutrophil; PLT, platelet; RBC, red blood cell; WBC, white blood cell; and WT, wild type.

TABLE 2
Complete Blood Counts in WT Littermate Control and Ero1α KO Mice.
Blood cells from mice were counted using HEMAVET 950 (Drew Scientific).

| | WBC, | NE, | LY, | MO, | RBC, | PLT, | MPV, |
|---|---|---|---|---|---|---|---|
| | 10⁹/L | 10⁹/L | 10⁹/L | 10⁹/L | 10¹²/L | 10⁹/L | fL |
| WT | 6.0 ± 1.2 | 0.8 ± 0.2 | 4.8 ± 1.1 | 0.2 ± 0.0 | 8.6 ± 2.0 | 1068 ± 220 | 4.6 ± 0.5 |
| Ero1α | 5.9 ± 1.1 | 0.8 ± 0.2 | 4.9 ± 0.9 | 0.2 ± 0.1 | 8.8 ± 1.2 | 905.3 ± 280 | 4.6 ± 0.4 |
| KO | | | | | | | |

The data represent the mean ± SD (n = 8-10 mice [8-12 wk old] per group). Ero1α indicates endoplasmic reticulum oxidoreductase 1α; KO, knockout; LY, lymphocyte; MO, monocyte; MPV, mean platelet volume; NE, neutrophil; PLT, platelet; RBC, red blood cell; WBC, white blood cell; and WT, wild type.

Atherothrombosis occurs after the rupture of an atherosclerotic plaque. To mimic the pathological condition, a FeCl₃-induced carotid arterial thrombosis model was utilized; an occlusive platelet thrombus is produced by application of the FeCl₃-saturated filter paper to the external surface of the carotid artery. Compared with WT control mice, Ero1α CKO and KO mice exhibited a significant prolongation of the time to occlusion in the injured carotid artery (FIG. 2I). Mice deficient in β3 integrin—a model of Glanzmann thrombasthenia—showed a maximal increase in the time to occlusion compared with WT mice. Importantly, megakaryocyte-specific or global deletion of Ero1α did not affect bleeding times and blood loss after amputating the tail tip, whereas deletion of β3 integrin markedly increased (FIG. 2J and FIG. 2K). Overall, these results indicate that platelet Ero1α promotes platelet thrombus formation in arterial thrombosis and that Ero1α may not affect hemostasis in mice.

### Intracellular ERO1α Promotes Platelet Activation and Aggregation Independently of PDI

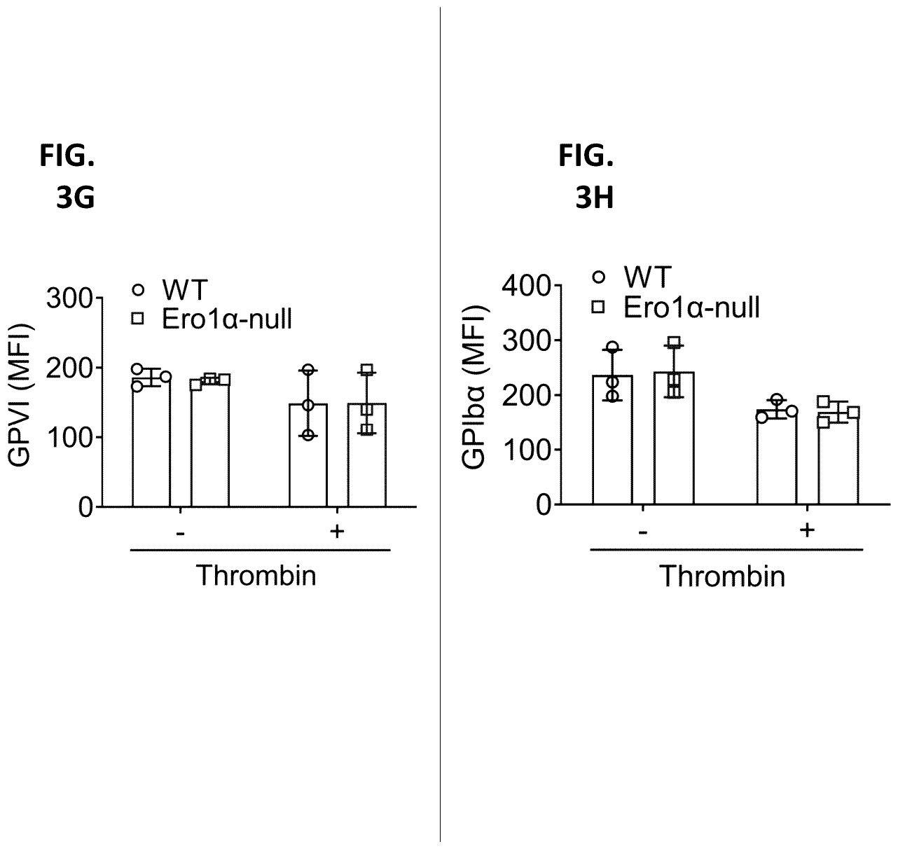

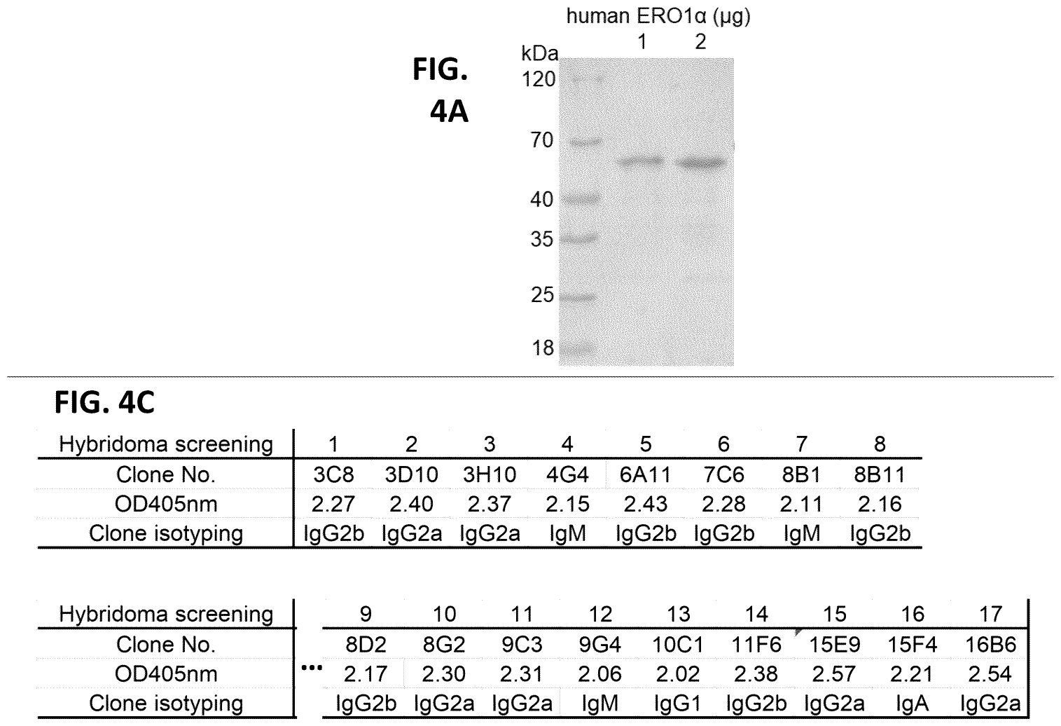

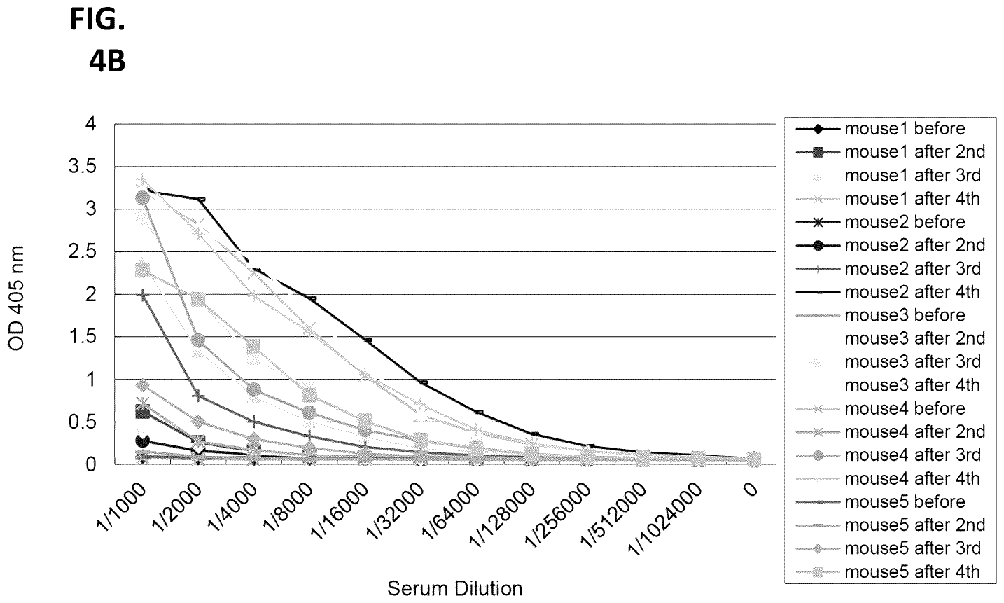

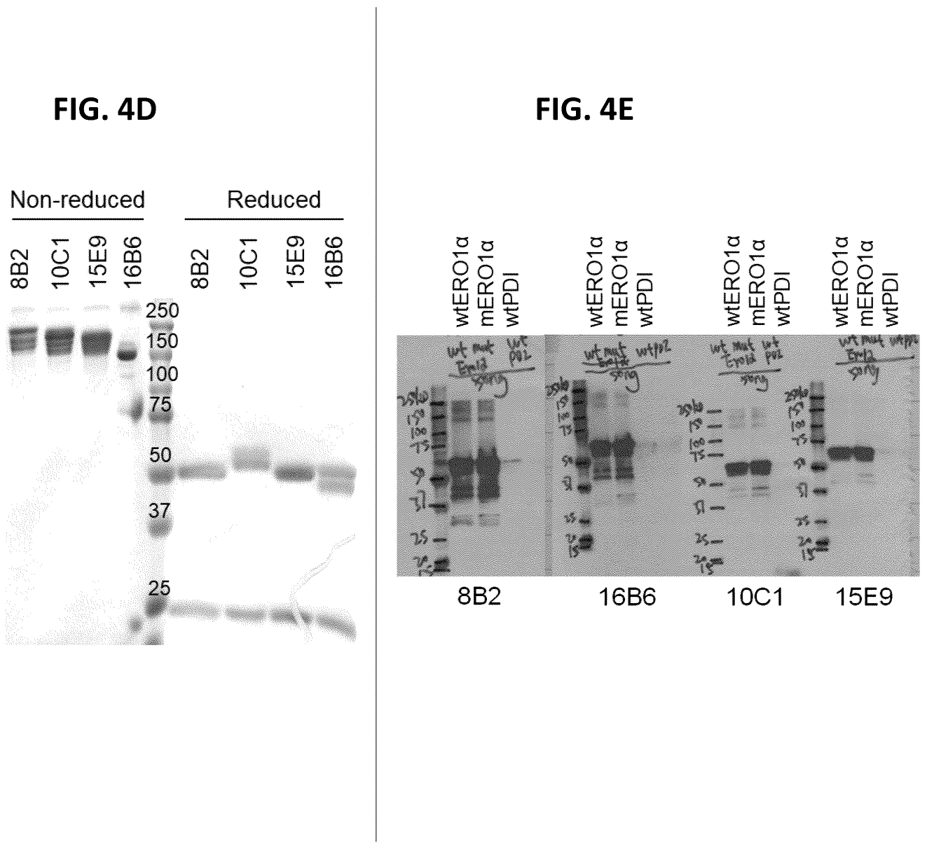

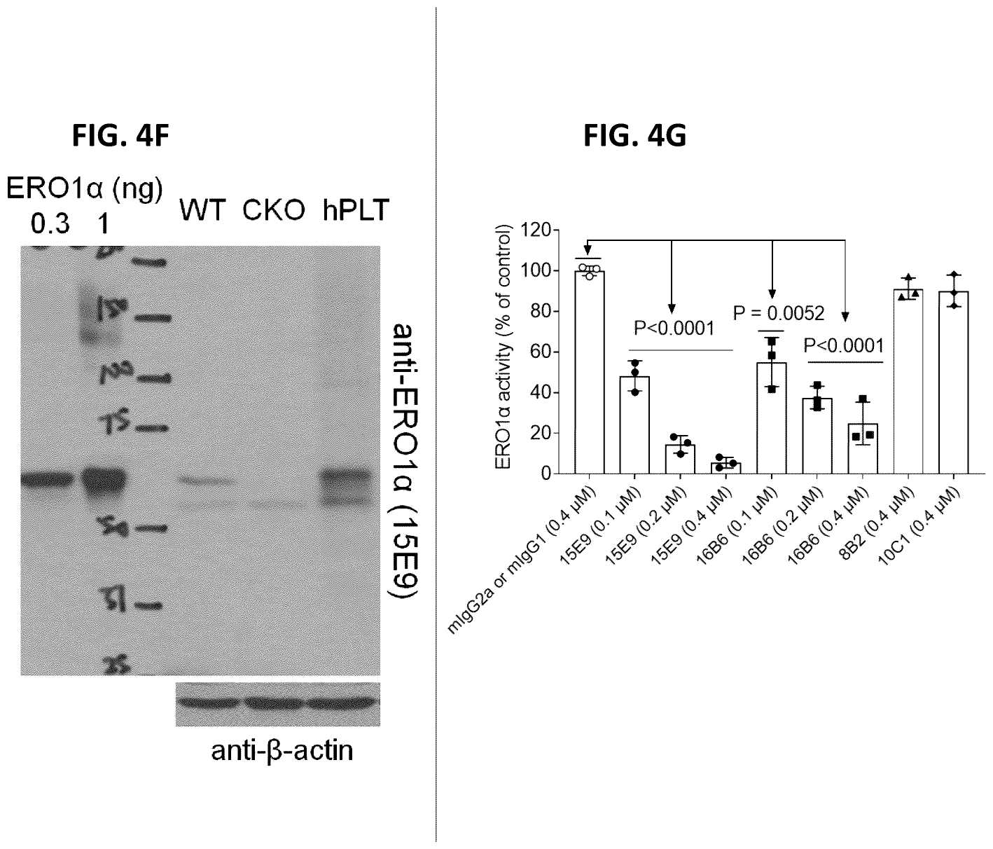

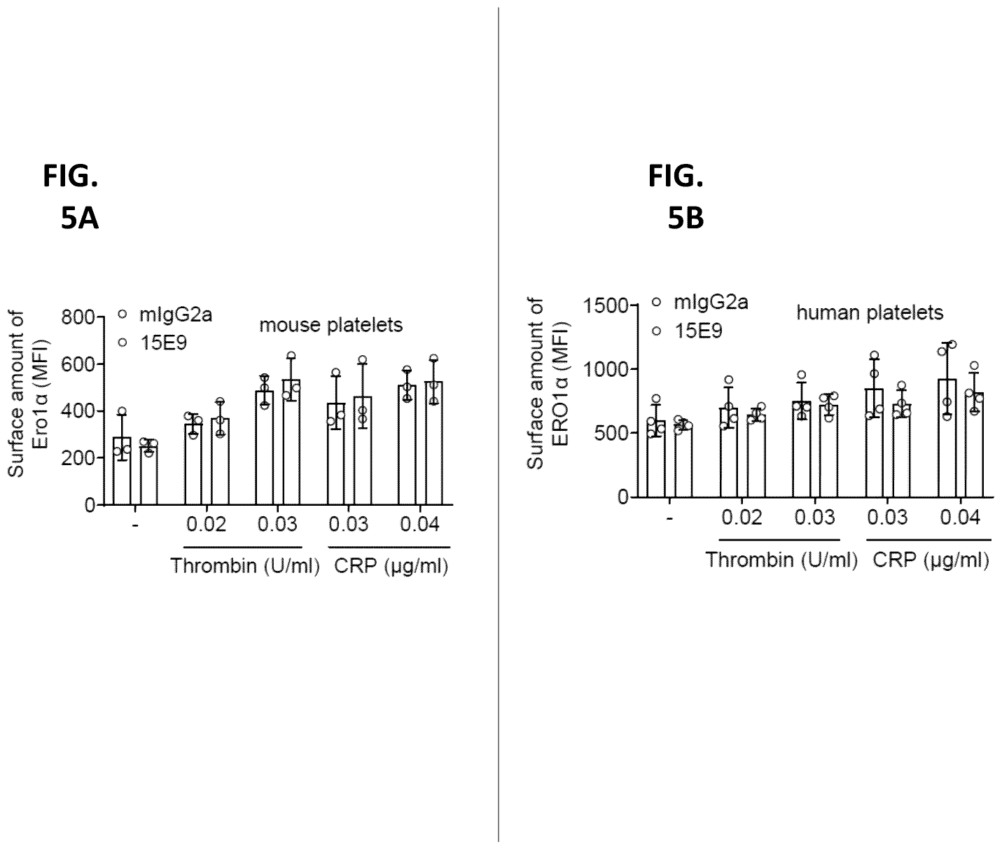

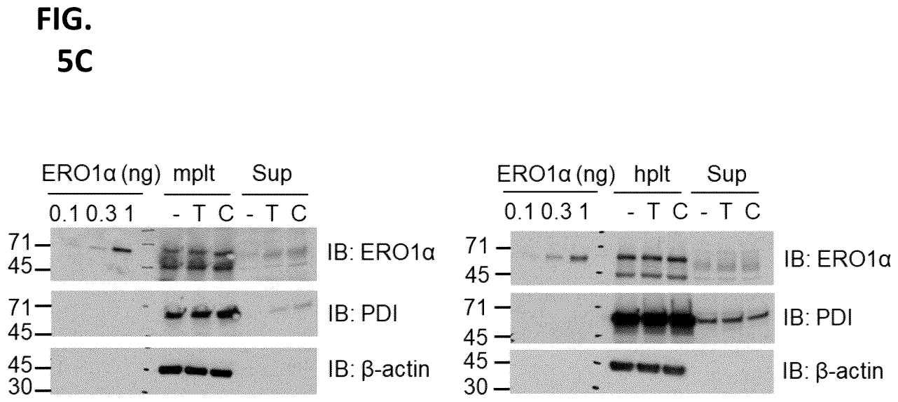

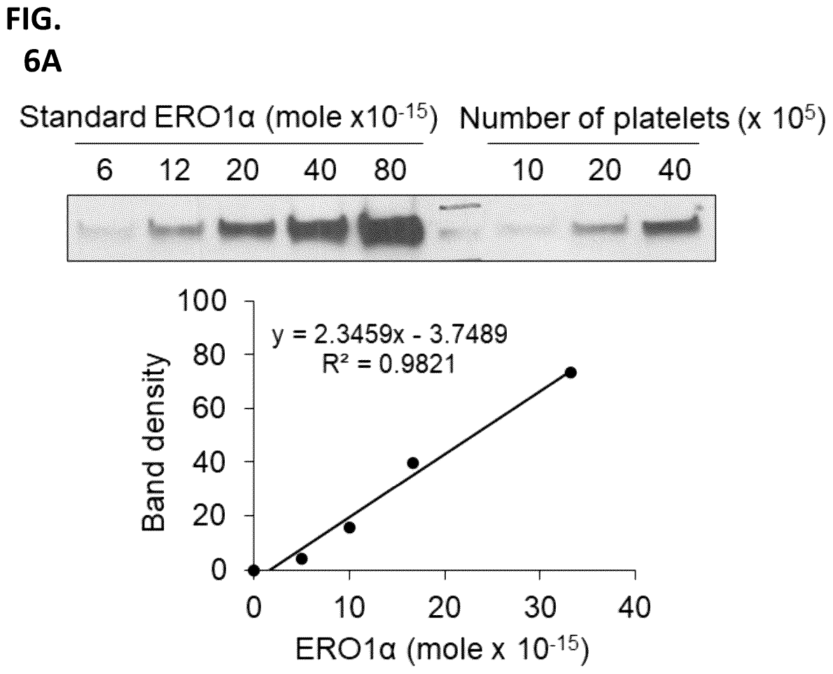

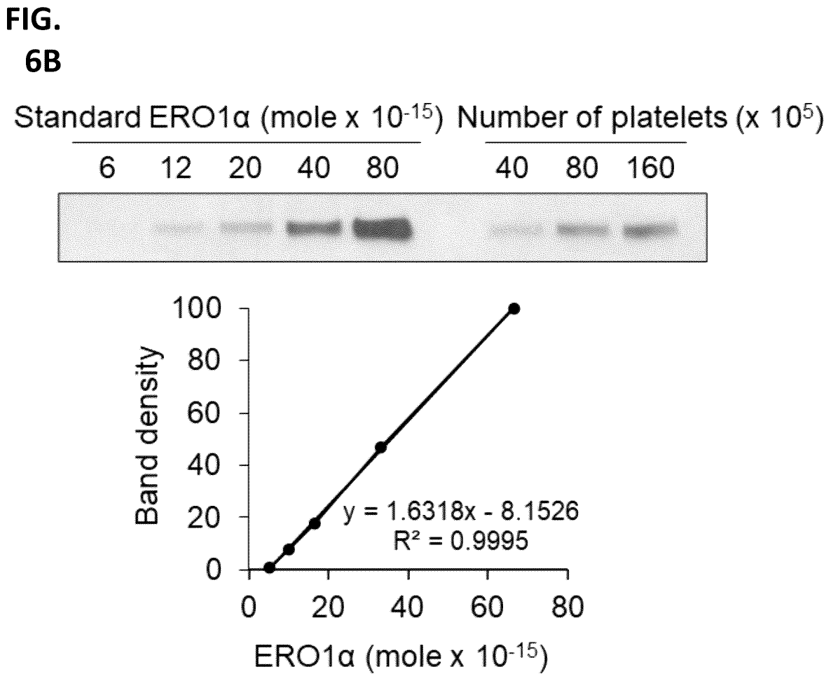

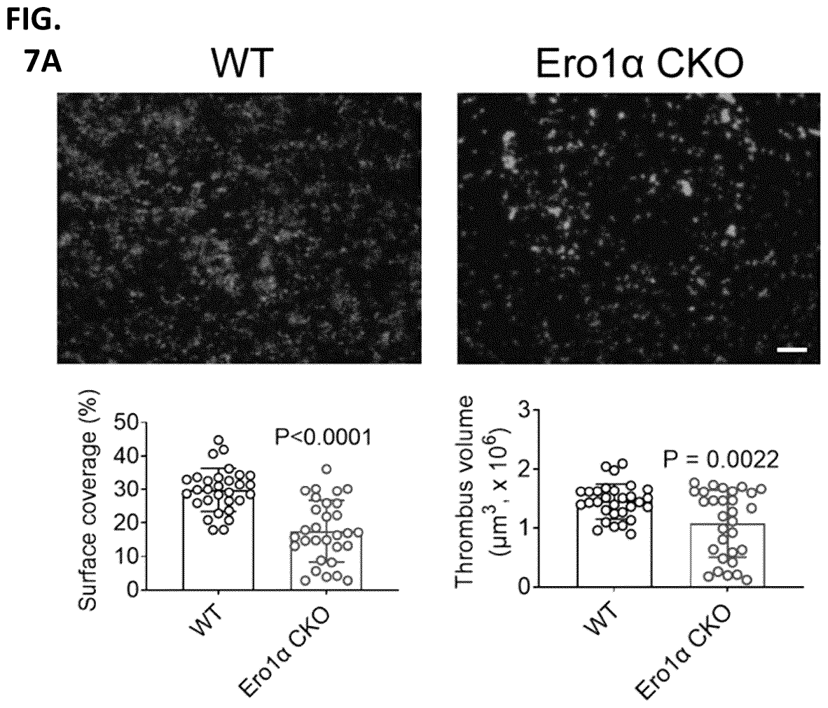

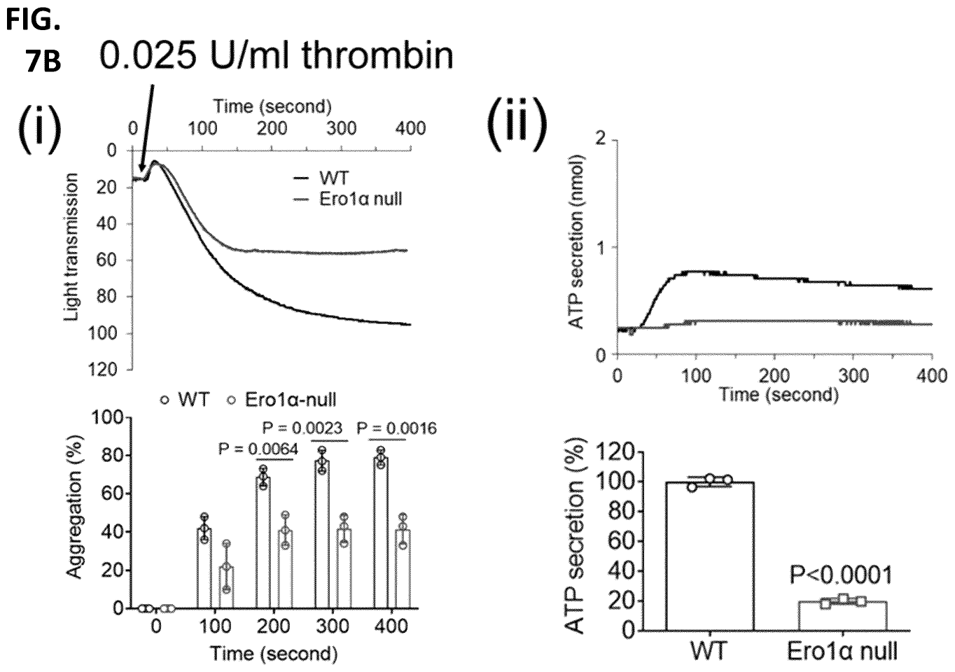

Due to the lack of inhibitory anti-ERO1α antibodies, mouse monoclonal blocking antibodies against recombinant human ERO1α were developed. After screening antisera and testing hybridomas (FIG. 4A through FIG. 4C), clone 15E9 was identified that recognizes human and mouse platelet ERO1α, wtERO1α, and mERO1α, but not PDI, and inhibits the oxidase activity of ERO1α in a concentration-dependent manner (FIG. 4D through FIG. 4G). 8B2 and 10C1 were identified as nonblocking anti-ERO1α antibodies. Flow cytometry showed no binding of 15E9 to mouse and human platelets under resting and activated conditions (FIG. 5A and FIG. 4B). Furthermore, unlike PDI, ERO1α was not detected in the releasate from activated mouse and human platelets (FIG. 5C). As previously reported, ≈32 000 PDI molecules are present in 1 human platelet and <10% of total PDI is released during platelet activation. Given the assumption that 15E9 binds to recombinant human ERO1α and human and mouse platelet ERO1α with the same affinity, it was found that 1 human or mouse platelet expressed ≈2500 or 1800 ERO1α molecules, respectively (FIG. 6(A-B)).

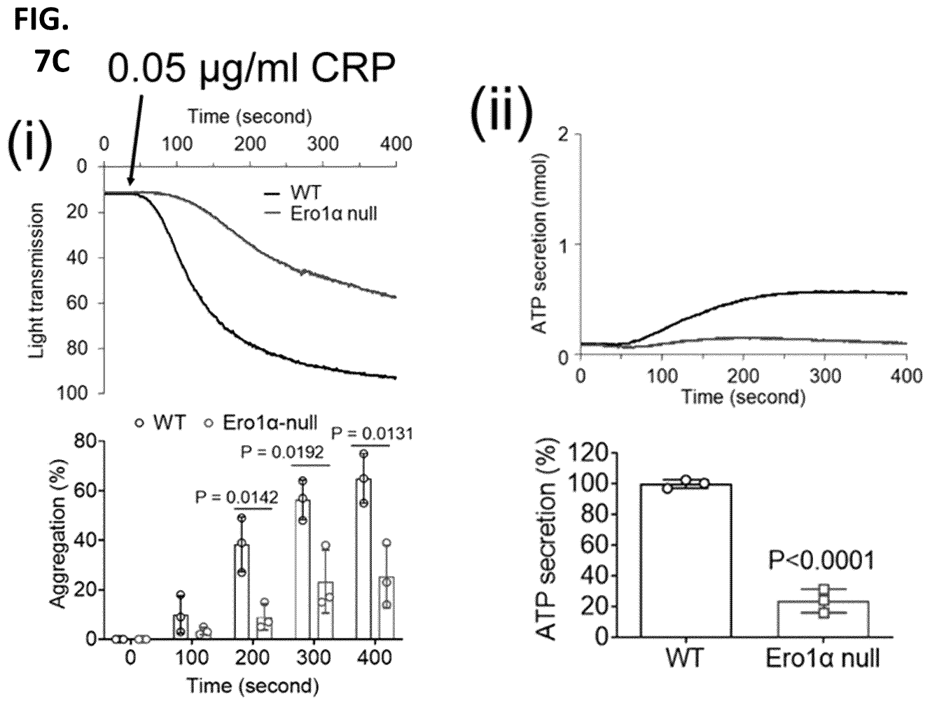

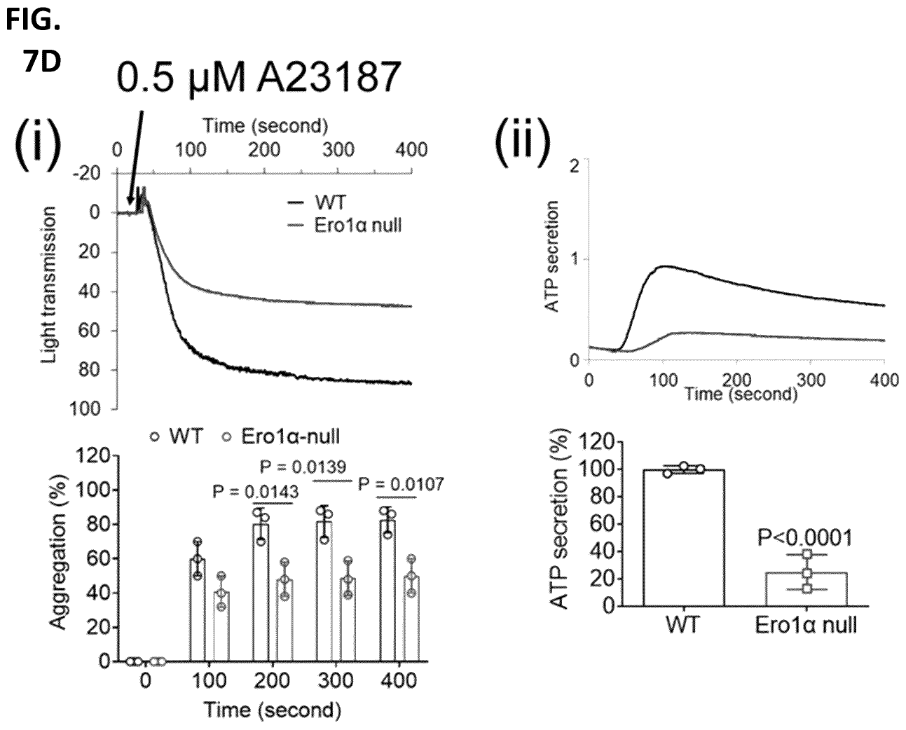

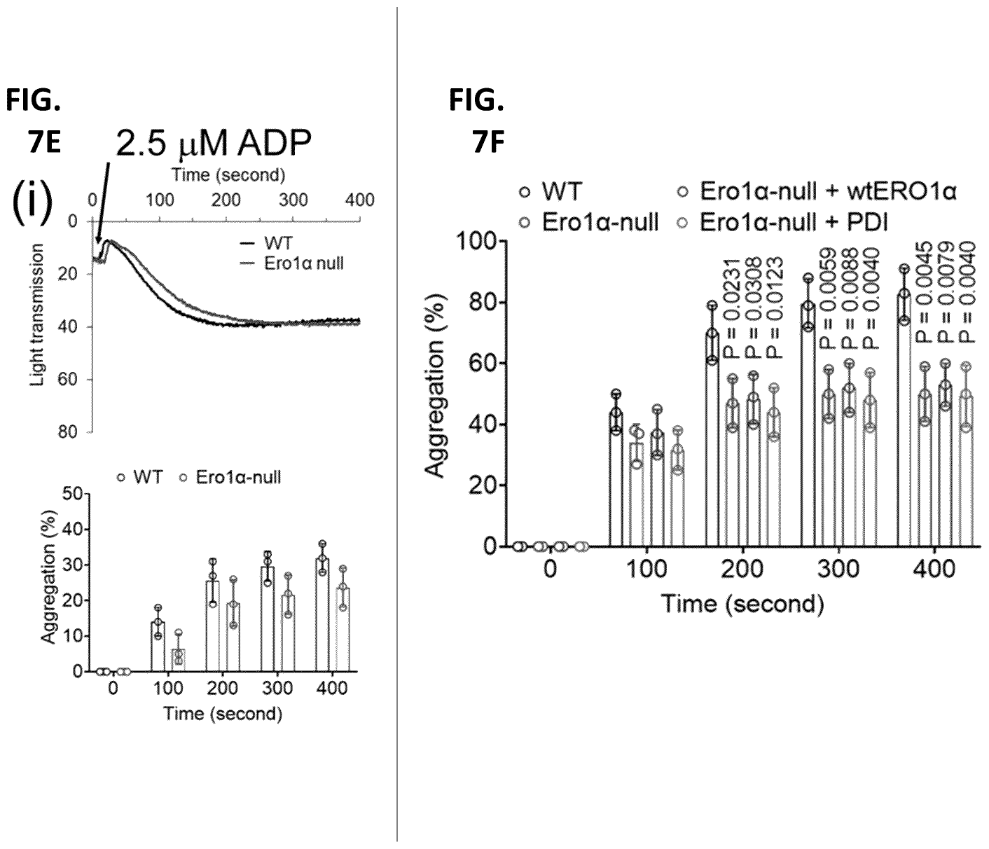

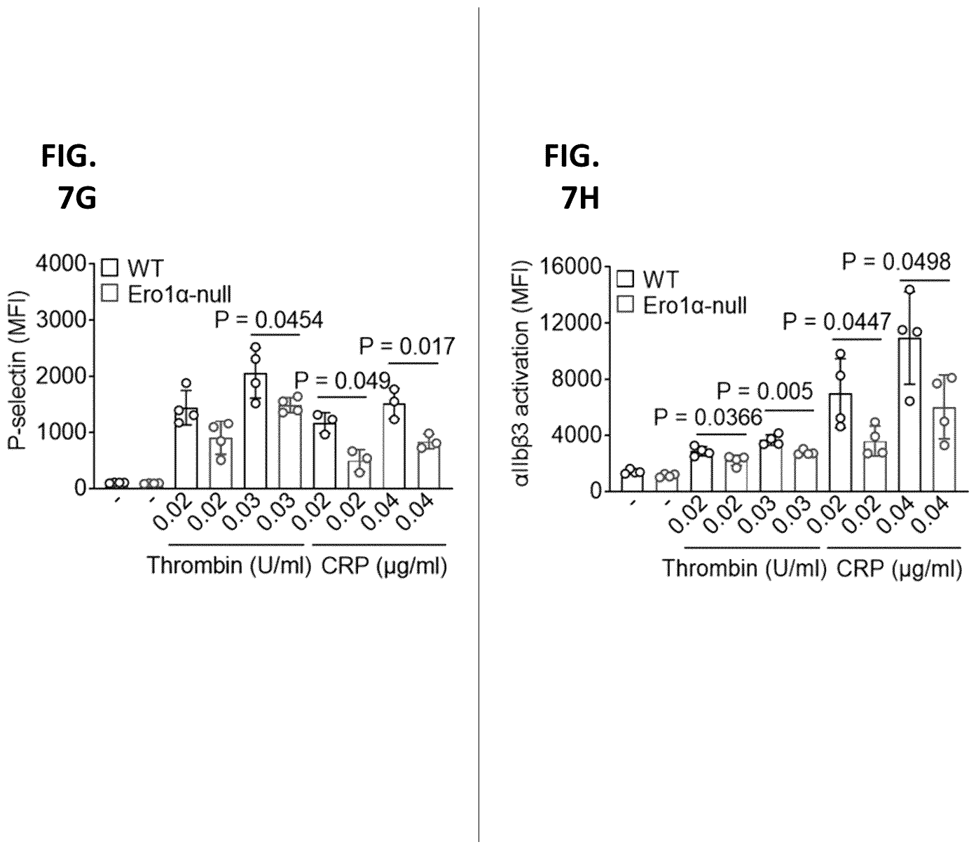

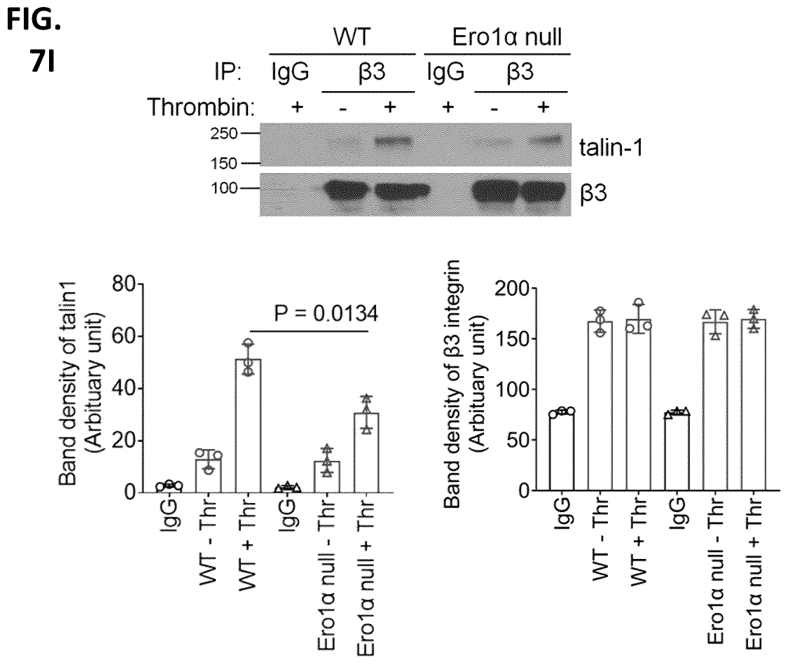

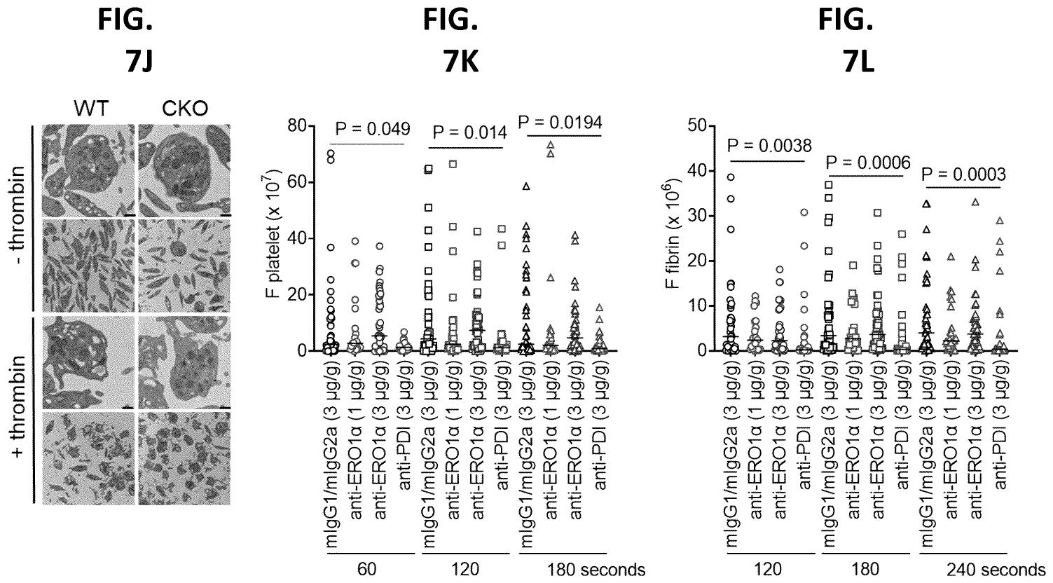

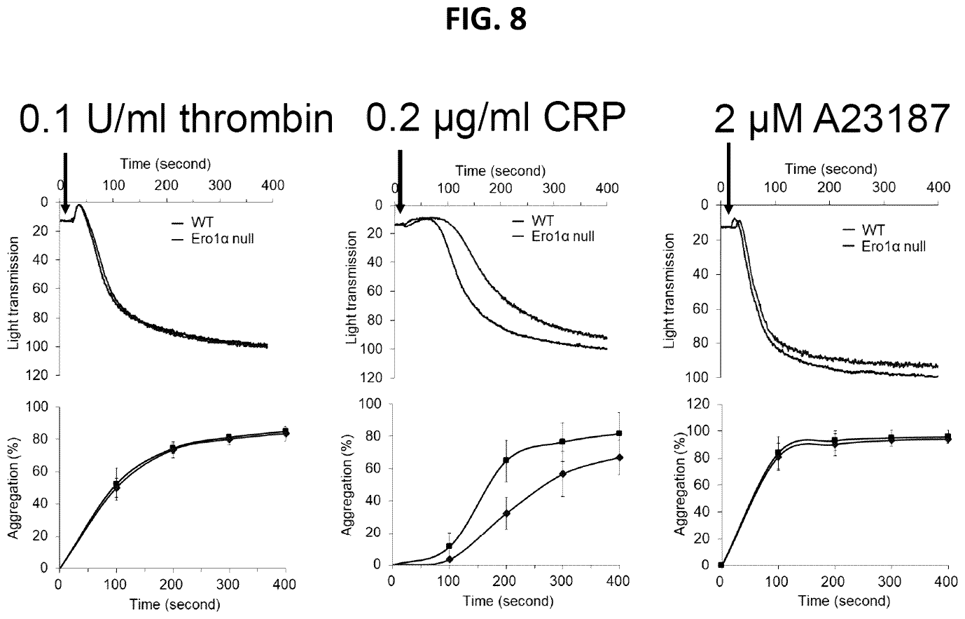

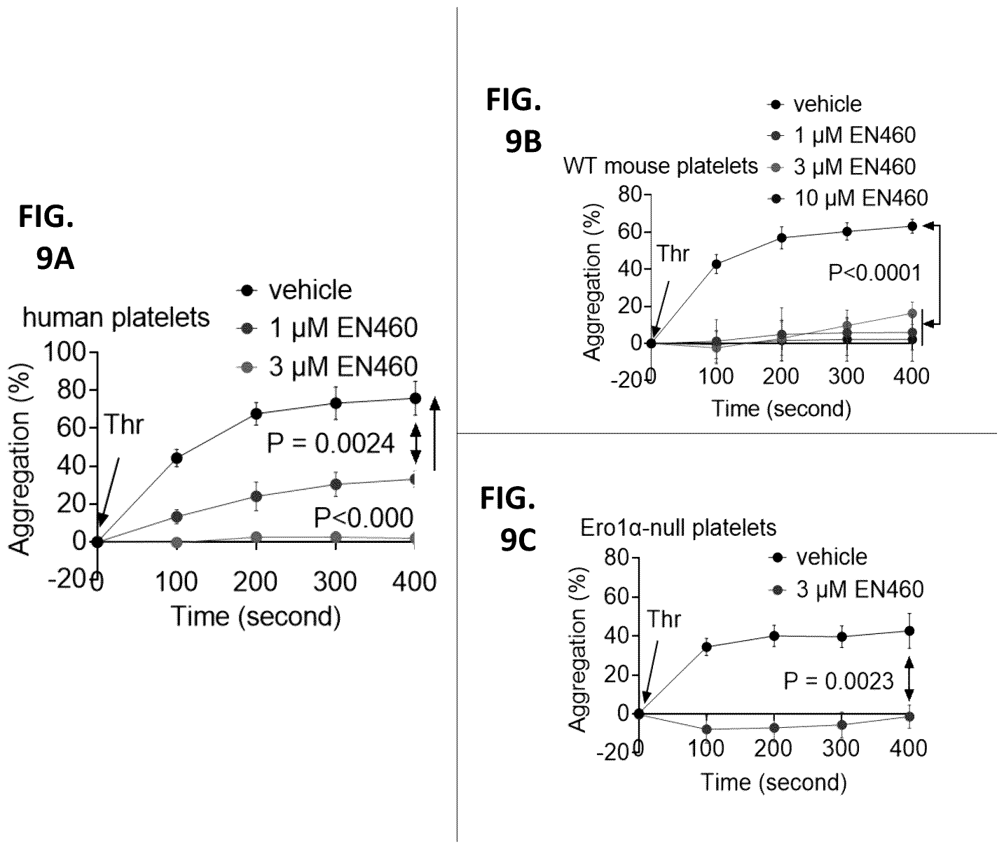

To confirm the contribution of platelet Ero1α to thrombus formation, a flow chamber assay was performed. Blood from WT and Ero1α CKO mice was perfused over immobilized fibrillar type I collagen at an arterial shear rate (1000 s⁻¹). WT platelets formed widespread thrombi on collagen surfaces, whereas Ero1α-null platelets showed a significant reduction in surface coverage and the volume of thrombi (FIG. 7A). As assessed by platelet aggregation and ATP (dense granule) secretion measured simultaneously by light transmission and luminescence, deletion of Ero1α significantly inhibited both events induced by various agonists, such as thrombin (0.025 U/mL), CRPs (collagen-related peptides; 0.05 μg/mL), and Ca² ionophore A23187 (0.5 μM) but not ADP (2.5 μM; FIG. 7B through FIG. 7E). Such defects disappeared when platelets were treated with a high concentration of agonists (FIG. 8). These results imply that platelet Ero1α regulates a common signaling pathway involved in Ca²⁺ signaling and granule secretion.

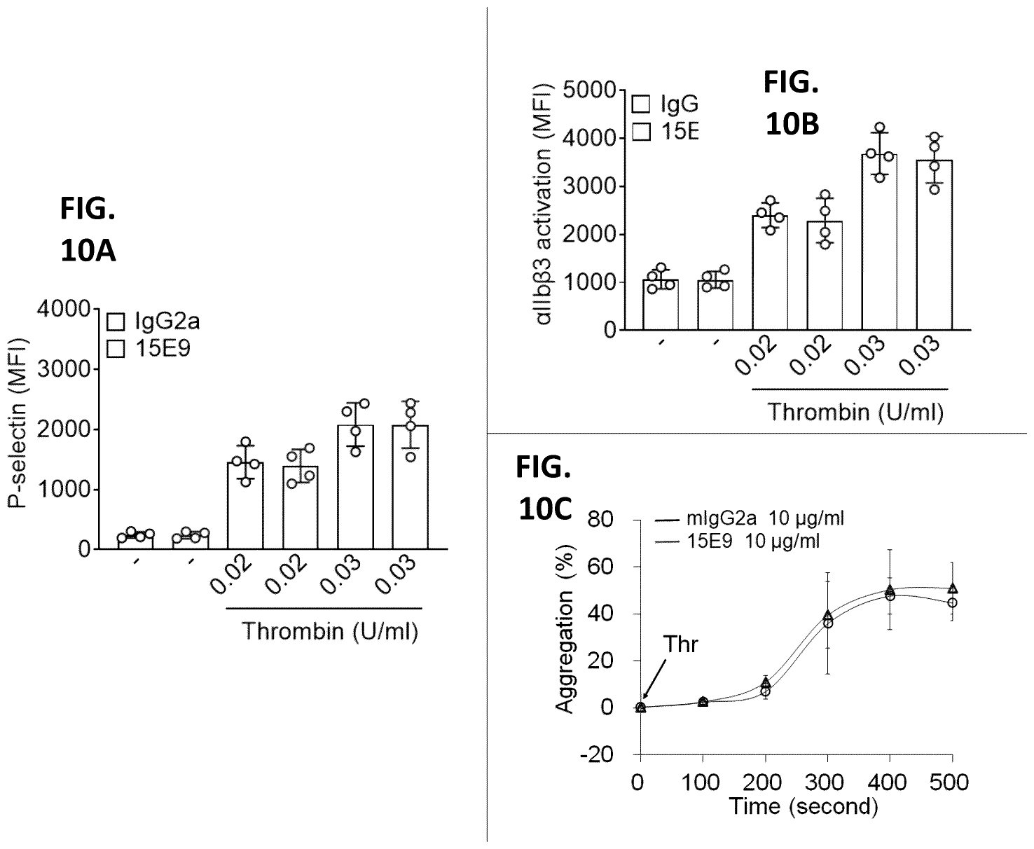

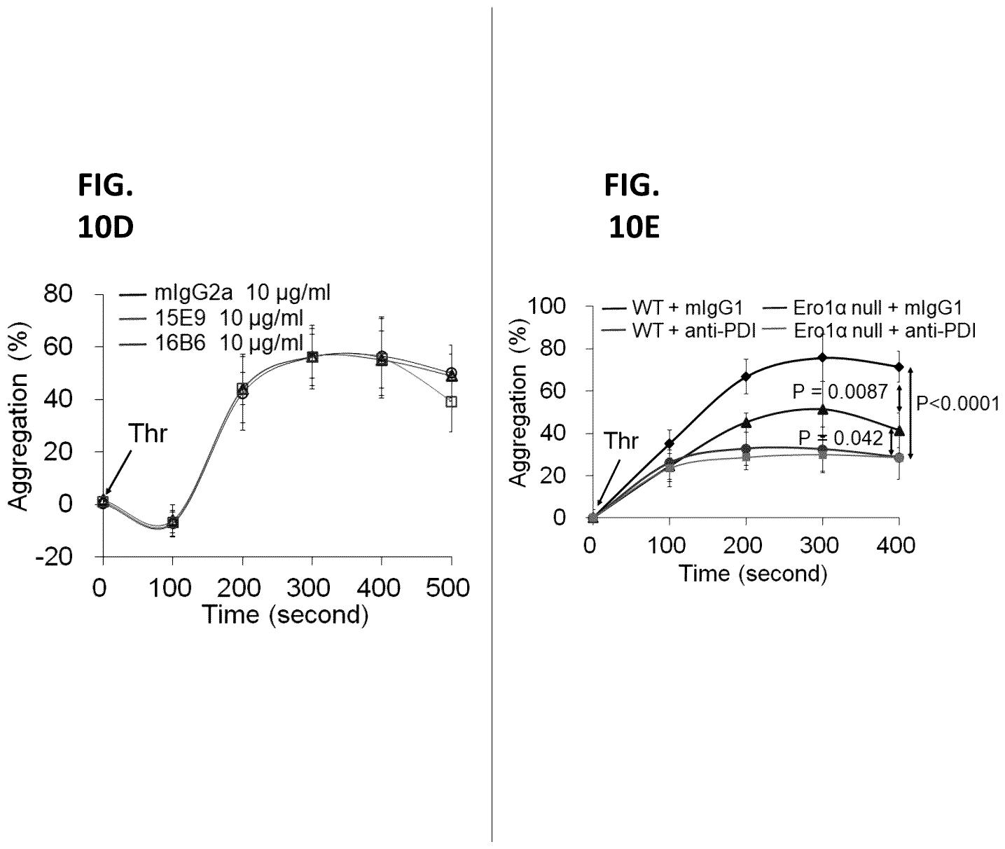

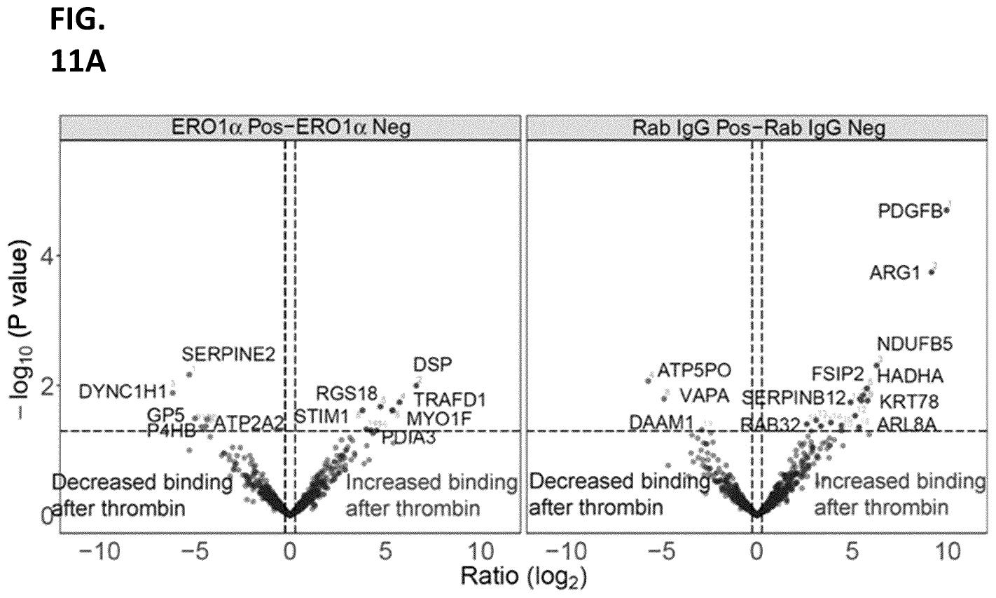

Pdi deletion reduces platelet aggregation and exogenously added wtPDI restores Pdi-null platelet aggregation to the WT level. However, treatment with wtERO1α or wtPDI did not rescue the defect in Ero1α-null platelet aggregation (FIG. 7F), suggesting that intracellular ERO1α promotes platelet aggregation independently of extracellular ERO1α and PDI. A recent study showed that 10 to 33 μM EN460, an ERO1α inhibitor (IC₅₀ [the half maximal inhibitory concentration], 1.9 μM), significantly blocks thrombin-induced platelet aggregation. From 3 to 10 μM EN460 abrogated thrombin-induced aggregation of mouse and human platelets (FIG. 9A and FIG. 9B). However, Ero1α-null platelet aggregation was also abolished by 3 μM EN460 (FIG. S7C).

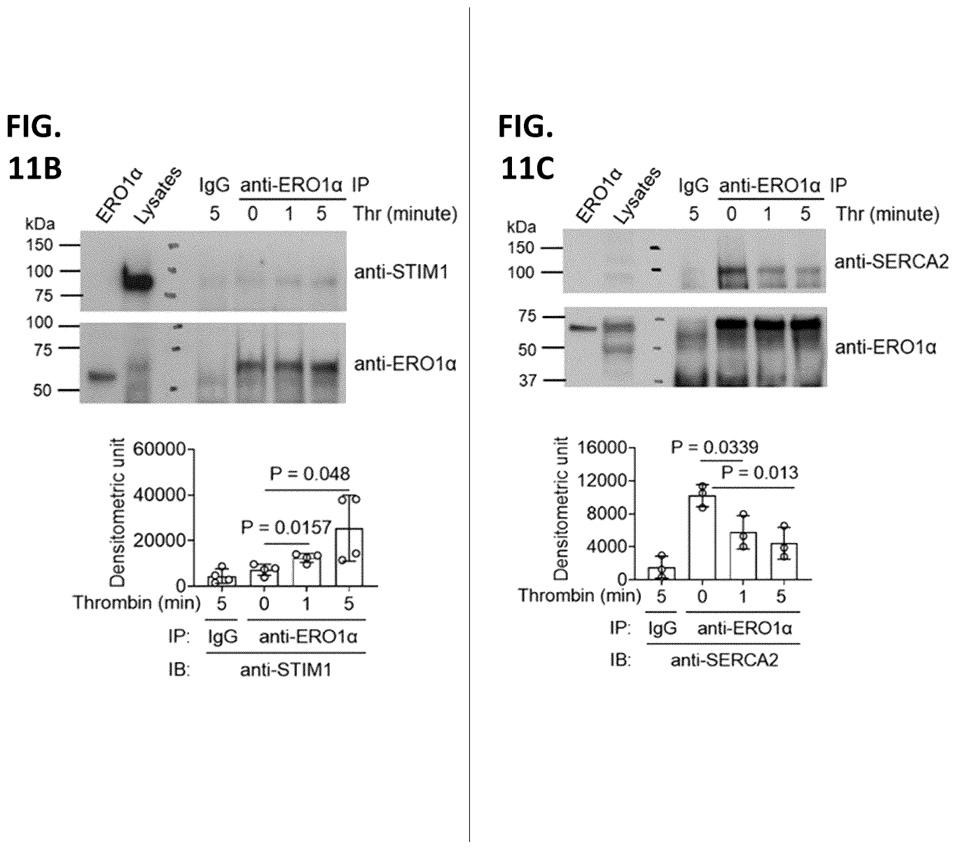

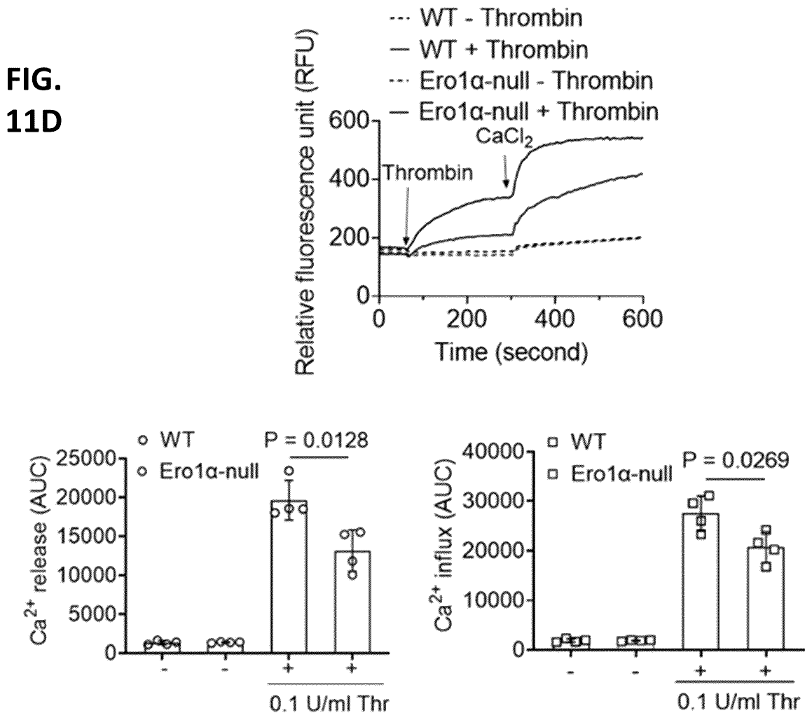

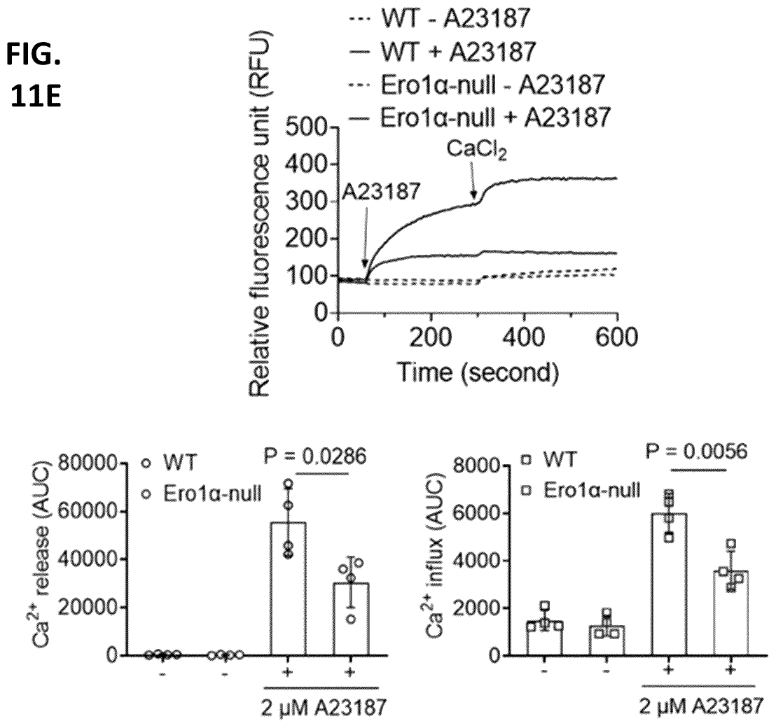

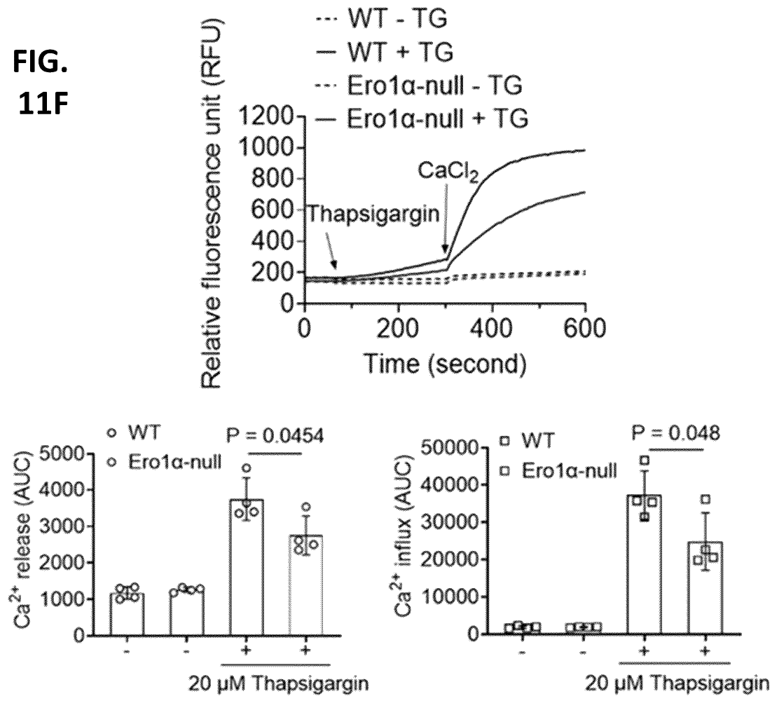

Loss of Pdi reduced αIIbβ3 integrin activation without affecting P-selectin exposure and the interaction between talin1 and the β3 integrin subunit. As measured by flow cytometry, loss of Ero1α impaired both P-selectin exposure and αIIbβ3 integrin activation in response to thrombin and CRP (FIG. 7G and FIG. 7H). Furthermore, immunoprecipitation assays showed that talin1-33 integrin binding was significantly reduced in thrombin-activated Ero1α-null platelets compared with WT platelets (FIG. 7I). Transmission electron microscopy revealed similar ultrastructures between WT and Ero1α-null platelets (FIG. 7J). Treatment with 15E9 did not affect P-selectin exposure and αIIbβ3 integrin activation in thrombin- or CRP-activated platelets (FIG. 10A and FIG. 10B). Also, inhibition of extracellular Ero1α with 15E9 did not impair thrombin-induced aggregation of mouse and human platelets (FIG. 10C and FIG. 10D). In contrast, treatment with a blocking anti-PDI antibody (BD34) inhibited aggregation of WT and Ero1α-null platelets (FIG. 10E). Using intravital microscopy, injection of 15E9 into mice did not reduce platelet thrombus formation and fibrin generation at the site of laser-induced cremaster arteriolar thrombosis (FIG. 7K and FIG. 7L). In contrast, inhibition of extracellular Pdi significantly blocked both events. Taken together, these results indicate that intracellular ERO1α promotes platelet activation and aggregation independently of PDI activity.

Platelet ERO1α Contributes to Ca²⁺ Content and Enhances Ca²⁺ Mobilization by Modifying an Allosteric Disulfide Bond in STIM1 and SERCA2

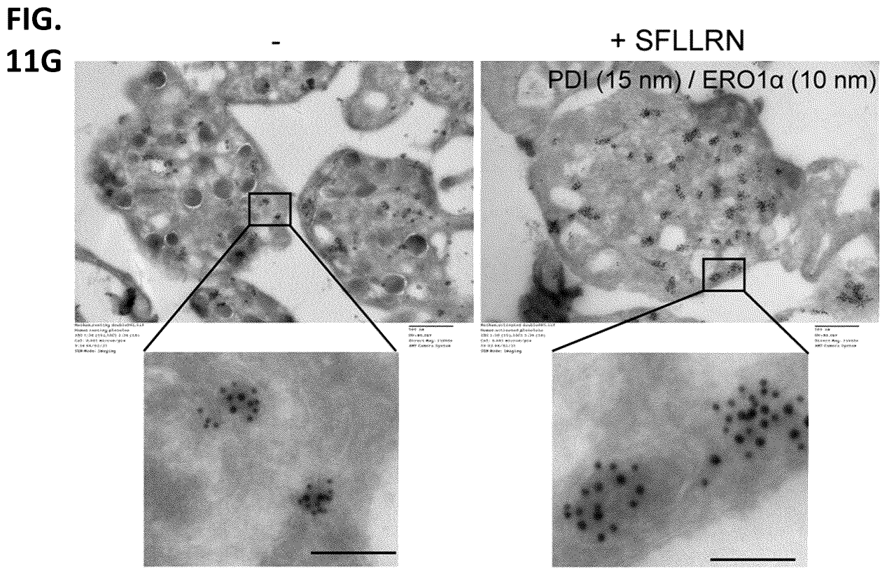

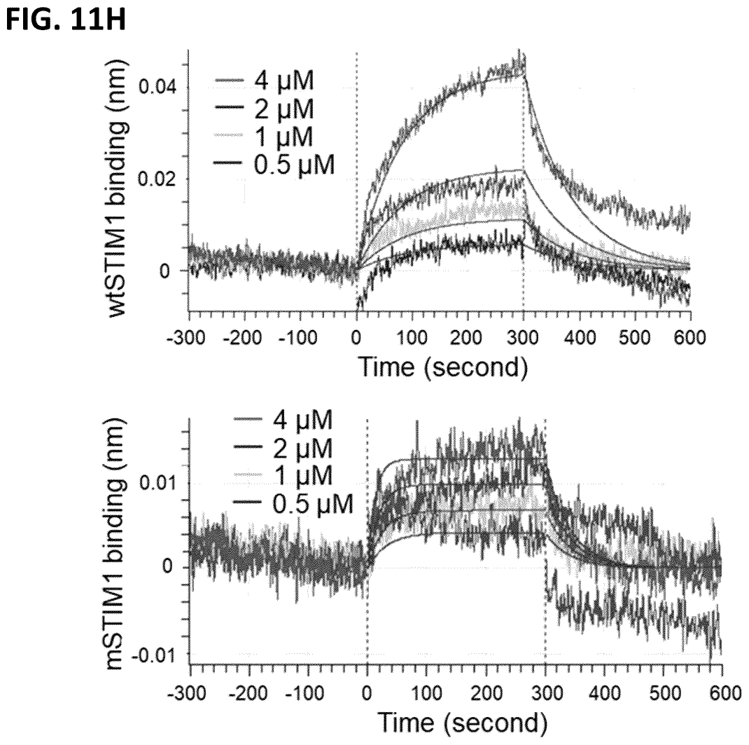

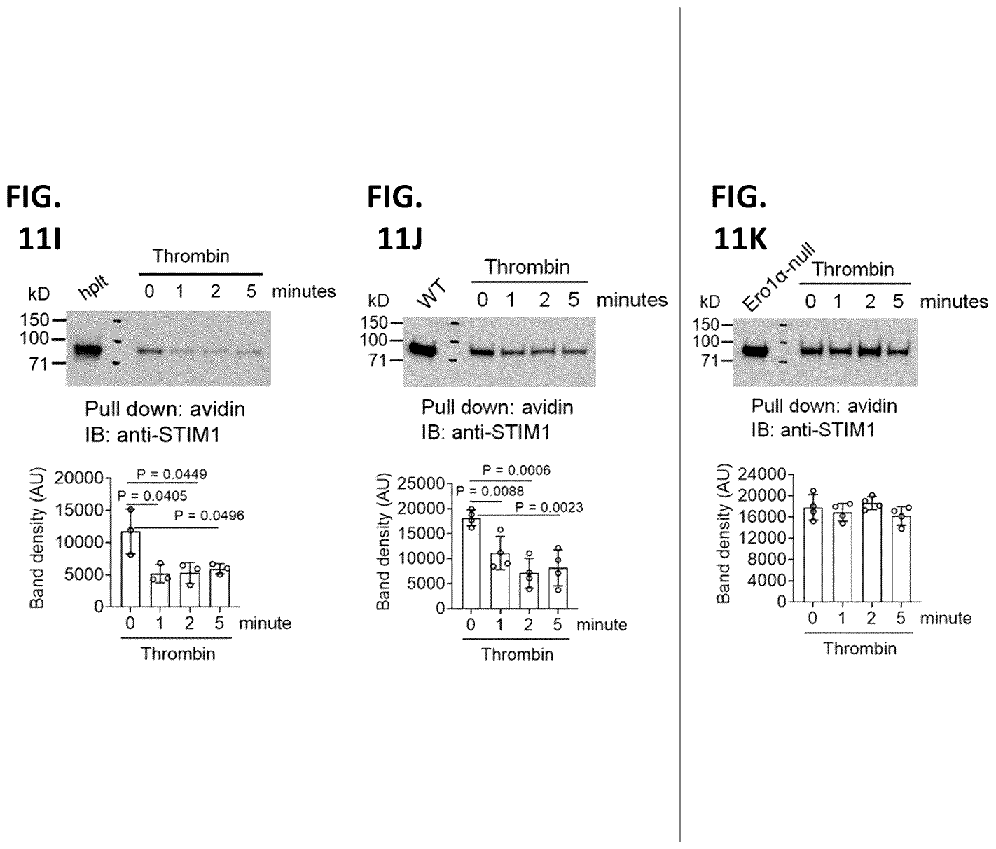

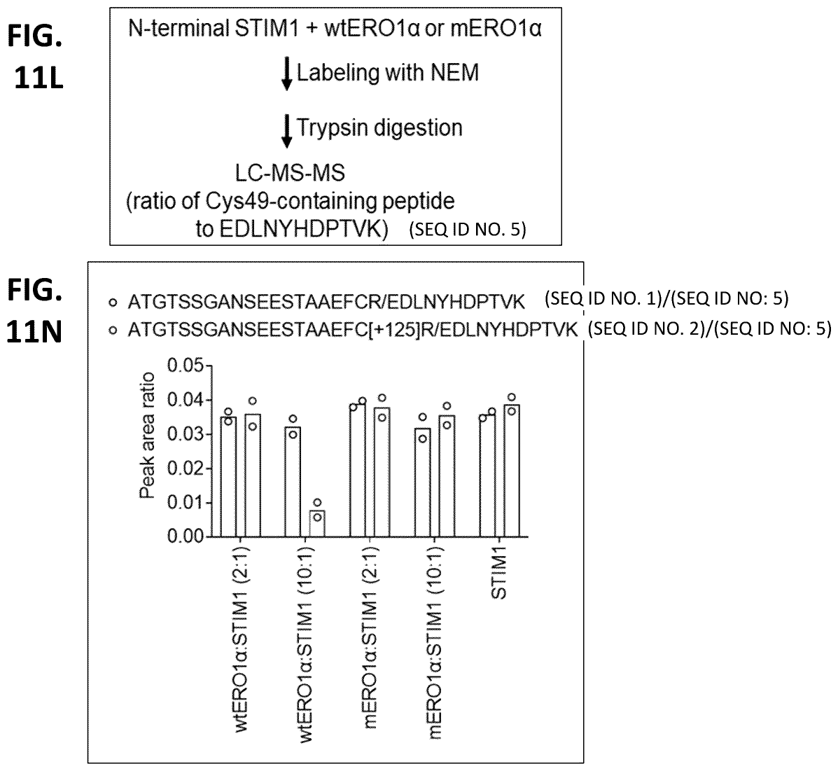

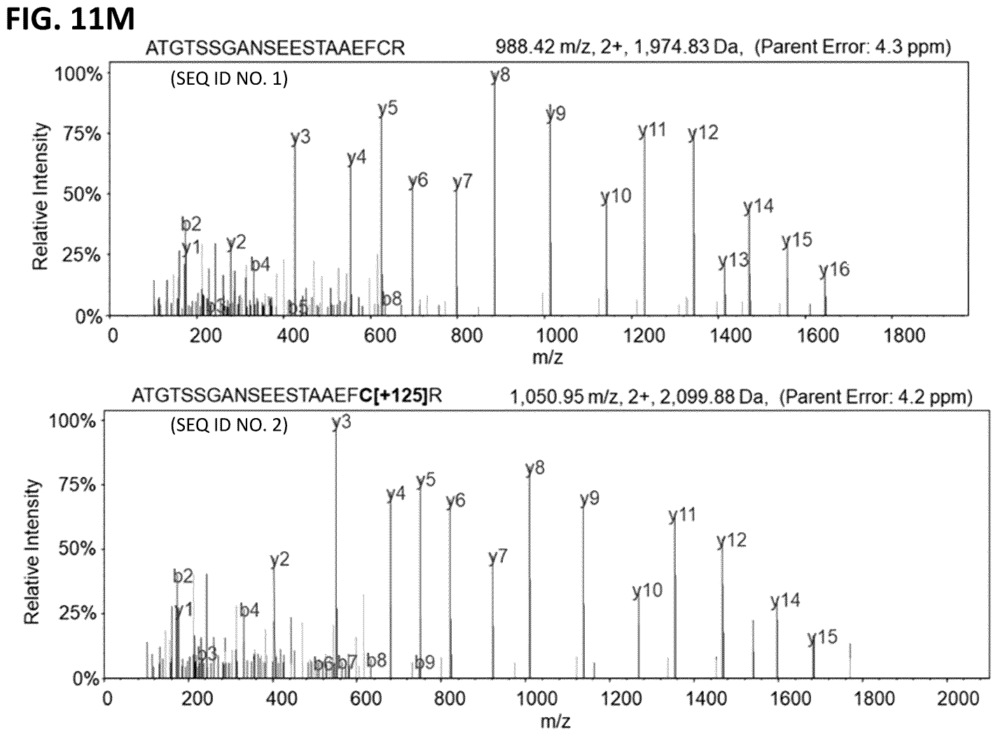

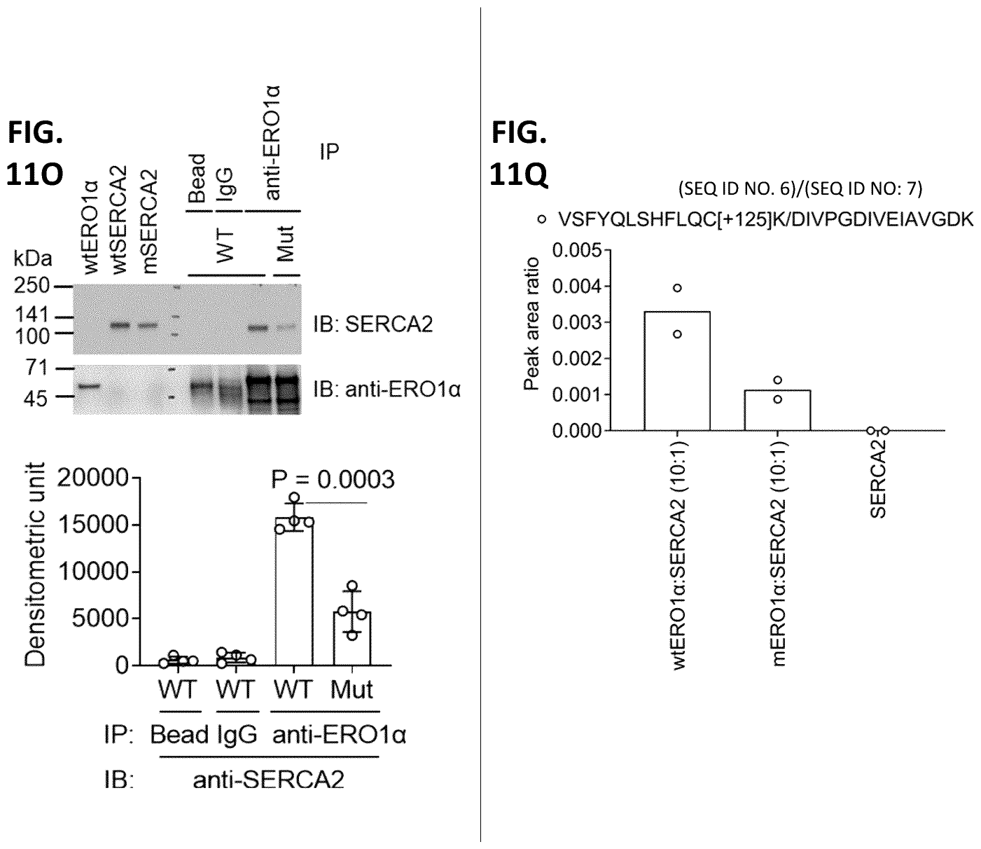

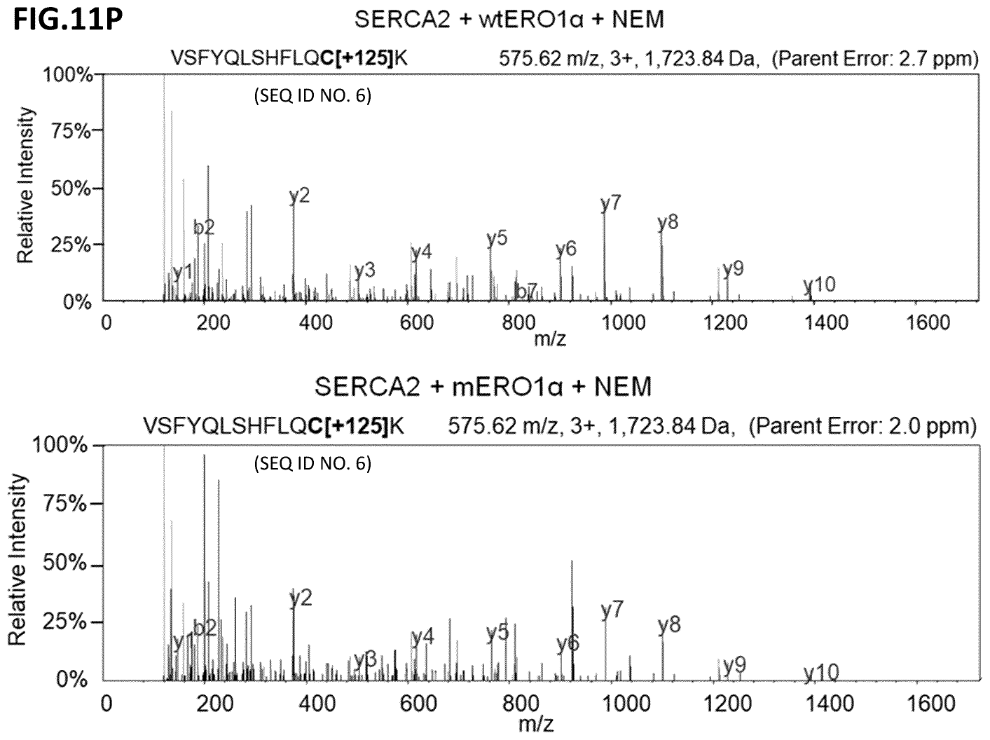

To determine how ERO1α regulates platelet function, mass spectrometry was first performed to identify its binding molecules. ERO1α interacted with many molecules, including PDI family member thiol isomerases, SERPINs, and Ca²⁺ signaling molecules, such as STIM1 and SERCA2 (FIG. 11A). The volcano plot showed a significant change in ERO1α binding to its binding molecules during platelet activation (FIG. 11A). Among them, focus was placed on STIM1 and SERCA2 since they are crucial for Ca²⁺ signaling, which may explain defects in Ero1α-null platelet activation and aggregation induced by multiple agonists. Using immunoprecipitation assays, it was confirmed that ERO1α bound to STIM1 and SERCA2 (FIG. 11B and FIG. 11C). Intriguingly, thrombin treatment significantly increased ERO1α binding to STIM1 but reduced its binding to SERCA2 in human platelets without affecting the ERO1α amount.

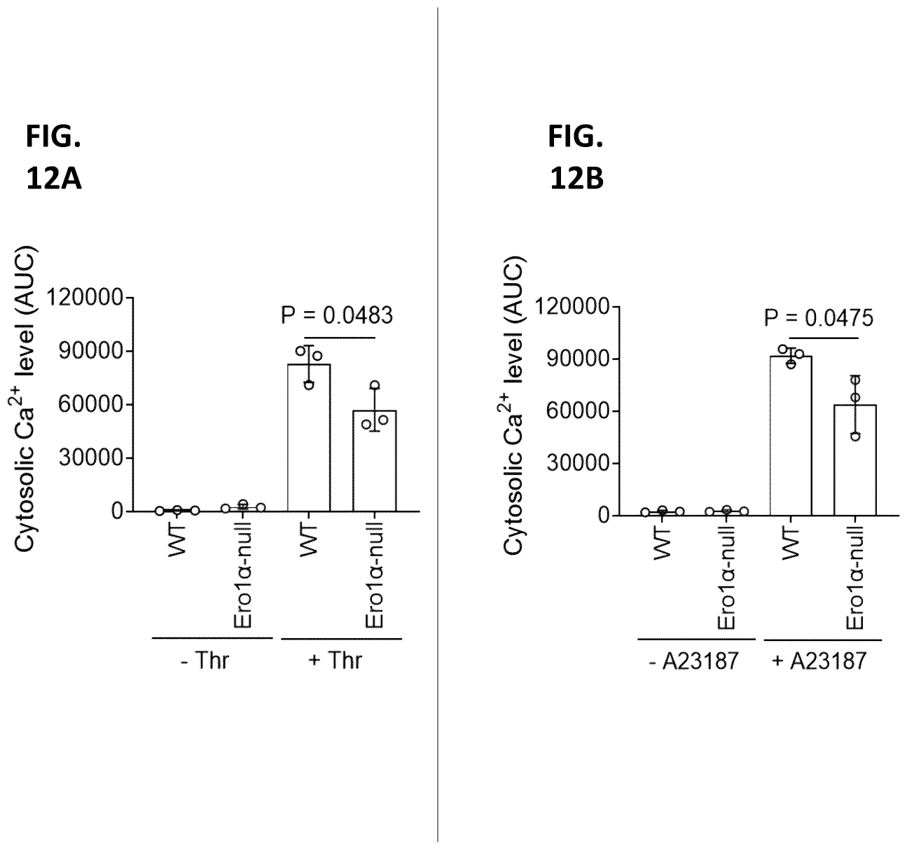

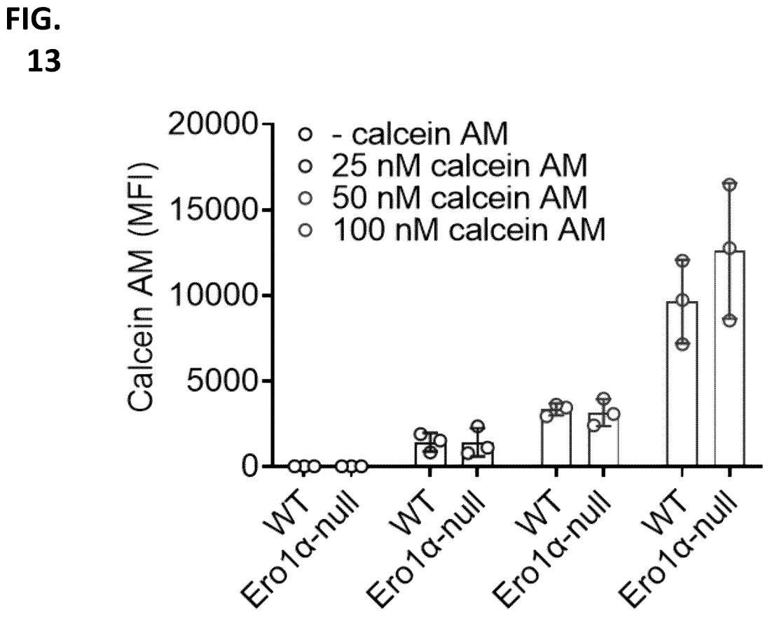

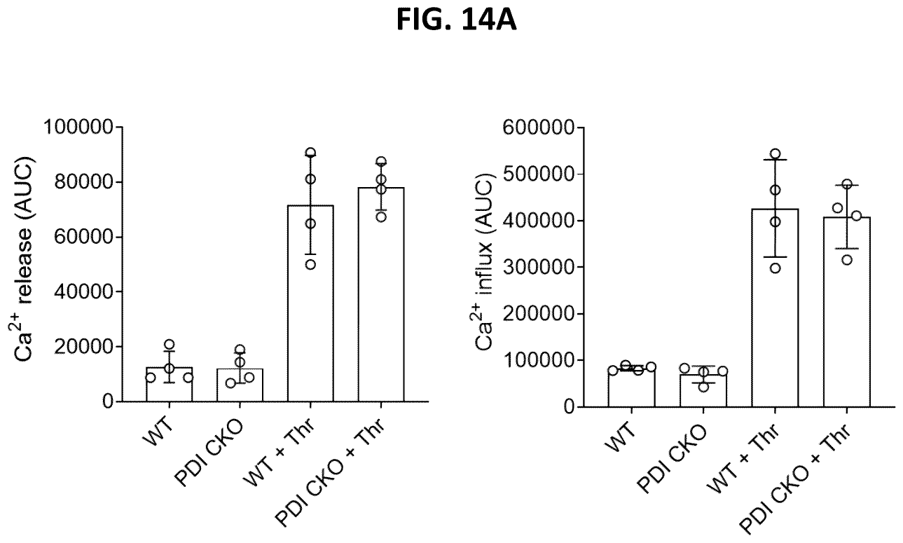

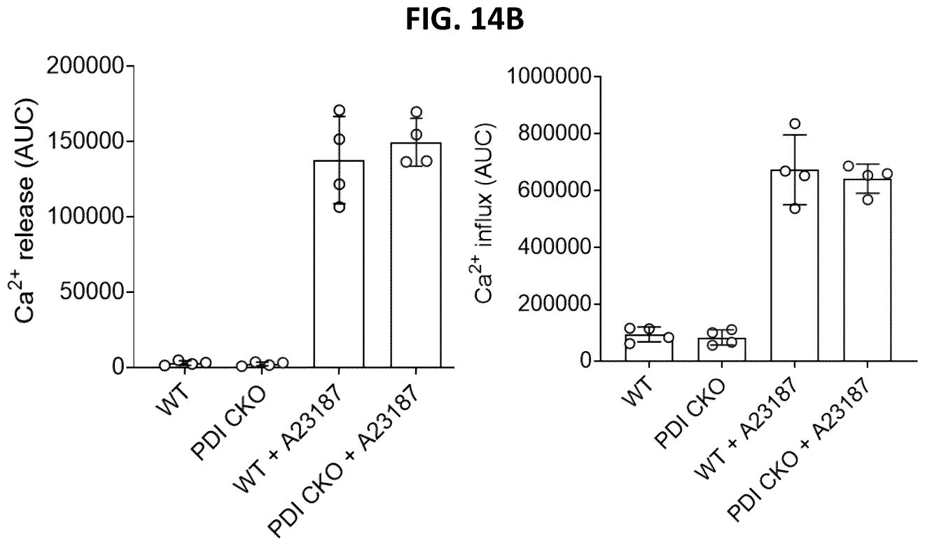

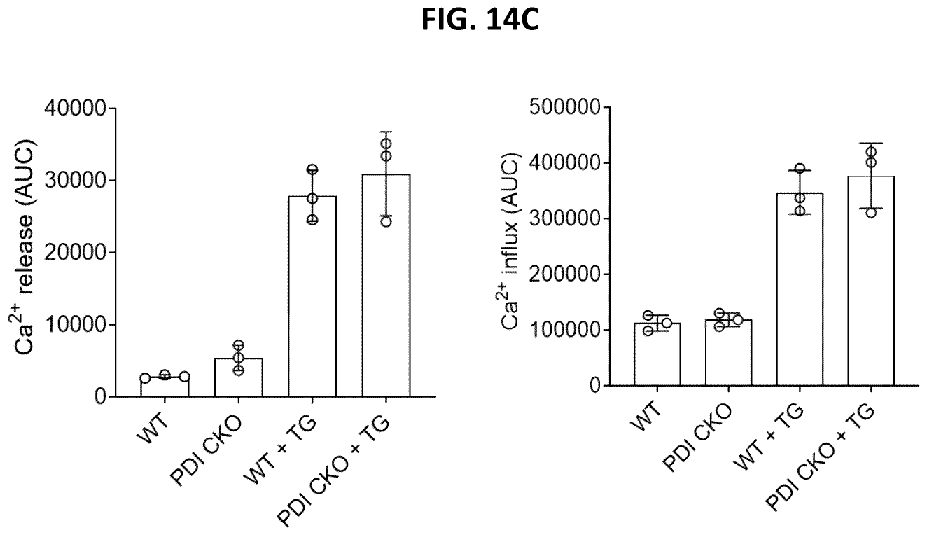

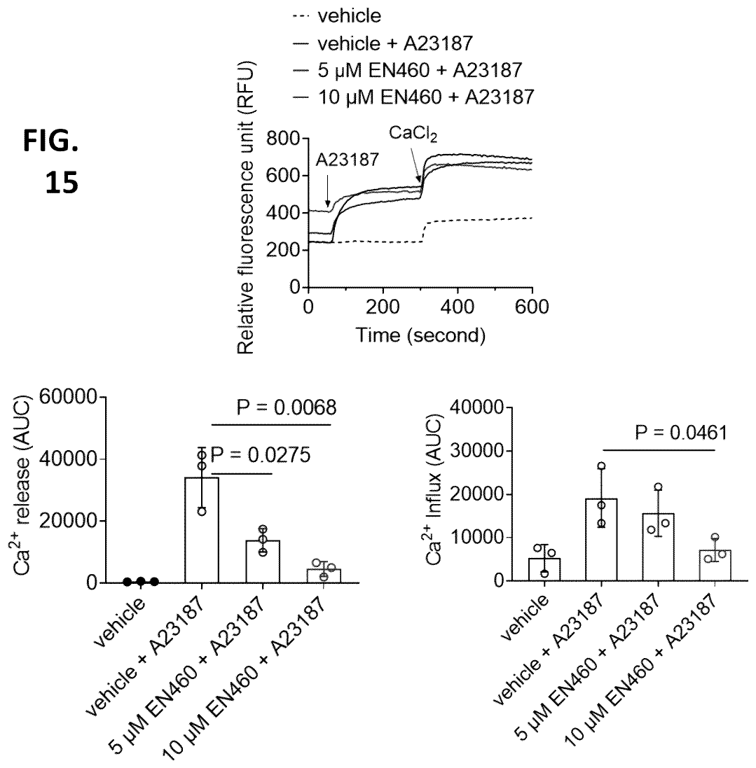

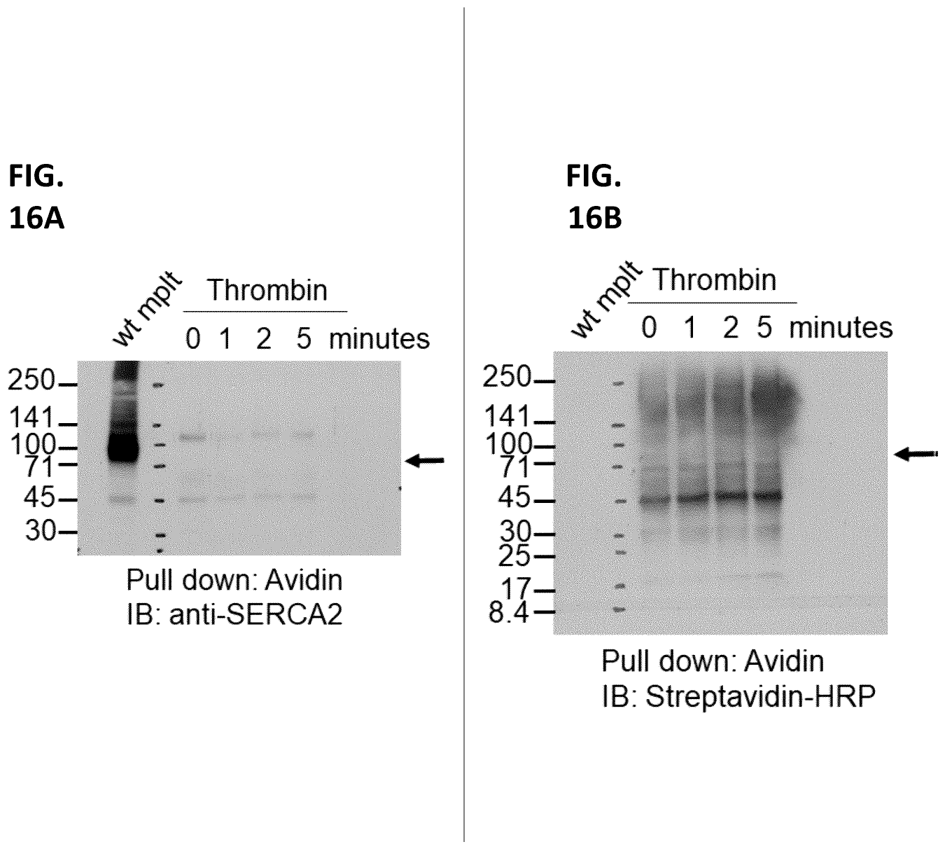

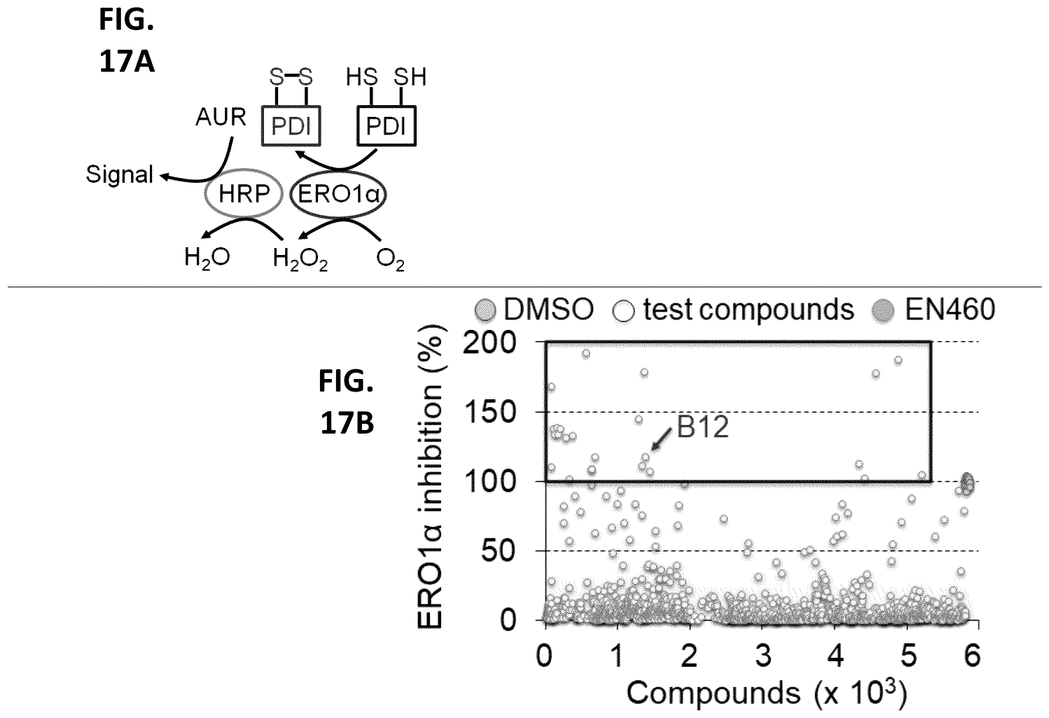

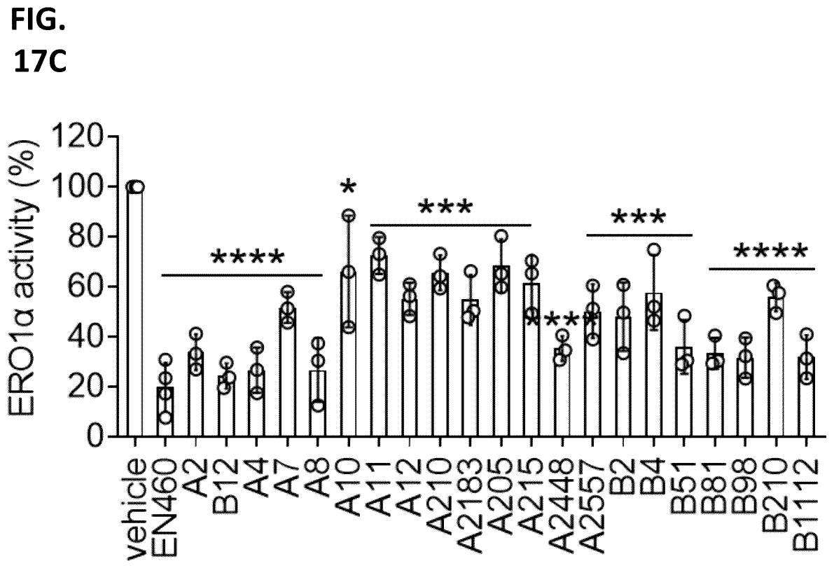

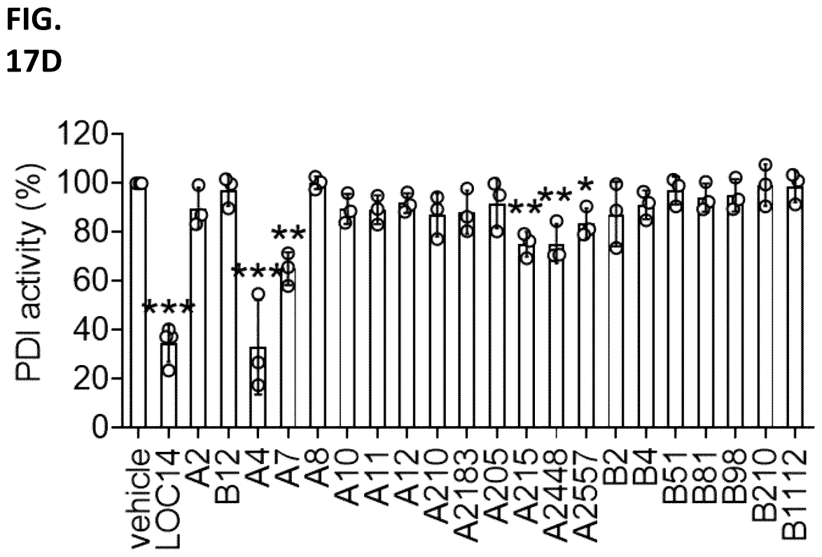

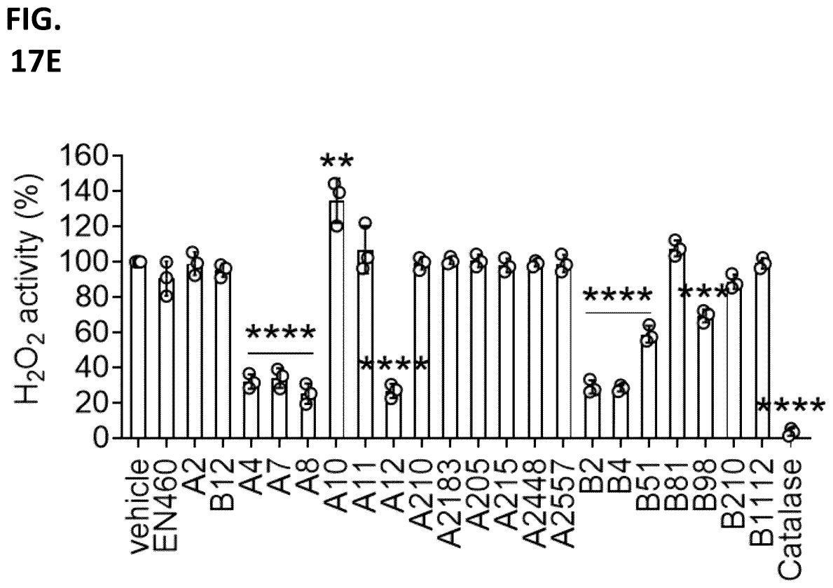

As assessed by the fluorescence-based FLIPR Ca²⁺ assay, compared with WT platelets, Ero1α-null platelets exhibited a significant decrease in both Ca²⁺ release and influx after stimulation with thrombin and A23187 (FIG. 11D and FIG. 11E). Similar results were obtained in the presence of CaCl₂ (FIG. 12(A-B)). Loss of platelet Ero1α reduced Ca²⁺ release and influx induced by thapsigargin, an inhibitor of SERCA (FIG. 11F). The decrease in agonist-induced Ca²⁺ mobilization in Ero1α-null platelets did not result from the alteration in dye loading or quenching since WT and Ero1α-null platelets were labeled with calcein AM equivalently (FIG. 13). Deletion of platelet Pdi did not affect agonist-induced Ca²⁺ mobilization (FIG. 14(A-C)). These results indicate that platelet Ero1α regulates Ca²⁺ store content and promotes SOCE in a PDI-independent manner. Although EN460 diminished Ca²⁺ release and influx upon A23187 stimulation, the compound increased the basal level of cytosolic Ca²⁺ in a concentration-dependent manner (FIG. 15).

PDI is reported to localize in the DTS of platelets. Immunogold electron microscopy using a monoclonal anti-PDI antibody (1D3) and a monoclonal anti-ERO1α antibody (10C1) revealed that ERO1α was found with PDI in the DTS in resting and activated human platelets, and no ERO1α was detected on the platelet surface (FIG. 11G). It is reported that deletion of platelet STIM1 reduces Ca²⁺ store content in the DTS and SOCE. Thus, ERO1α regulation of STIM1 function was investigated. Bio-layer interferometry was first performed using a biosensor coupled with biotinylated ERO1α and a different concentration of WT or mutant STIM1 in which Cys49 and Cys56 residues were mutated to Ser49 and Ser56 (mutant STIM1 [mSTIM1]-Cys49/56Ser). ERO1α directly bound to wtSTIM1 in a concentration-dependent manner, with a K_{D} value of 2.6 μM, whereas binding of ERO1α to mutant STIM1 was minimal and not concentration dependent (FIG. 11H), providing no meaningful K_{D} value. These results indicate that the Cys49 and Cys56 residues in STIM1 are critical for ERO1α binding.

S-nitrosylation at Cys49 and Cys56 residues induces a conformational change in STIM1 and suppresses Ca²⁺ depletion-dependent oligomerization, suggesting that a Cys49-Cys56 disulfide bond is likely to have an allosteric function. Thus, ERO1α was hypothesized to regulate the Cys49-Cys56 disulfide bond in STIM1 and its function. Using a pull-down assay with Na-(3-maleimidylpropionyl) biocytin reacting with free thiols, the level of free thiol groups in STIM1 was decreased in human and WT mouse platelets upon thrombin stimulation (FIG. 11I and FIG. 11J). However, such a decrease in the free thiol level was not observed in activated Ero1α-null platelets (FIG. 11K). Mass spectrometry revealed that wtERO1α, but not mERO1α, decreased N-ethylmaleimide labeling on the Cys49 residue in STIM1 in a concentration-dependent manner (FIG. 11L through FIG. 11N). However, the trypsin-digested Cys56-containing peptide, IDKPLCHSEDEK, was not detected probably due to the presence of acidic amino acids, which might affect trypsin digestion. The presently disclosed results provide the first evidence that ERO1α directly interacts with STIM1 and oxidizes the allosteric Cys49-Cys56 disulfide bond, contributing to Ca²⁺ store content and promoting SOCE during platelet activation.

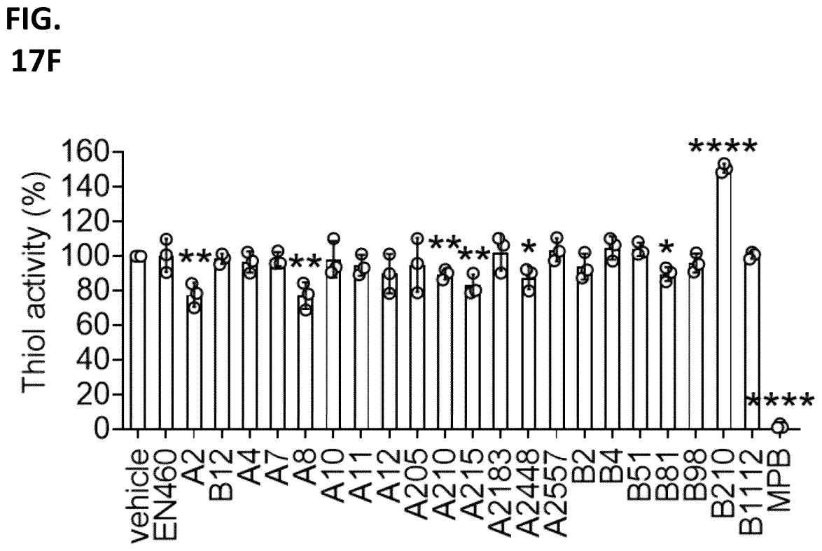

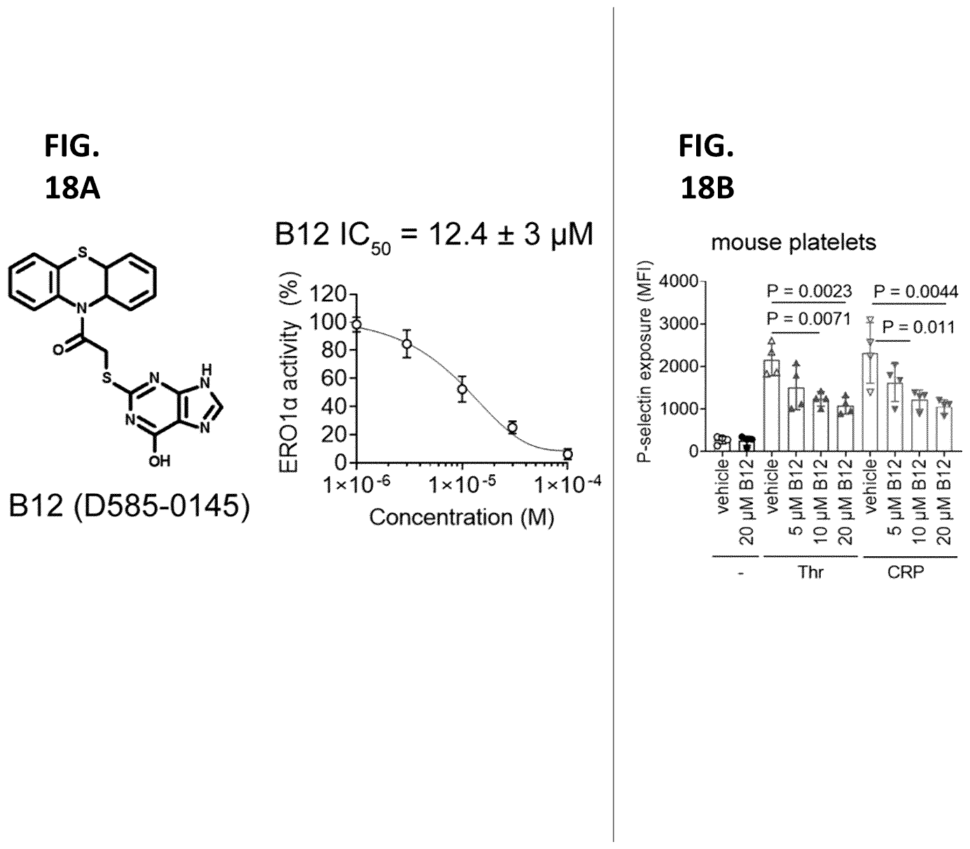

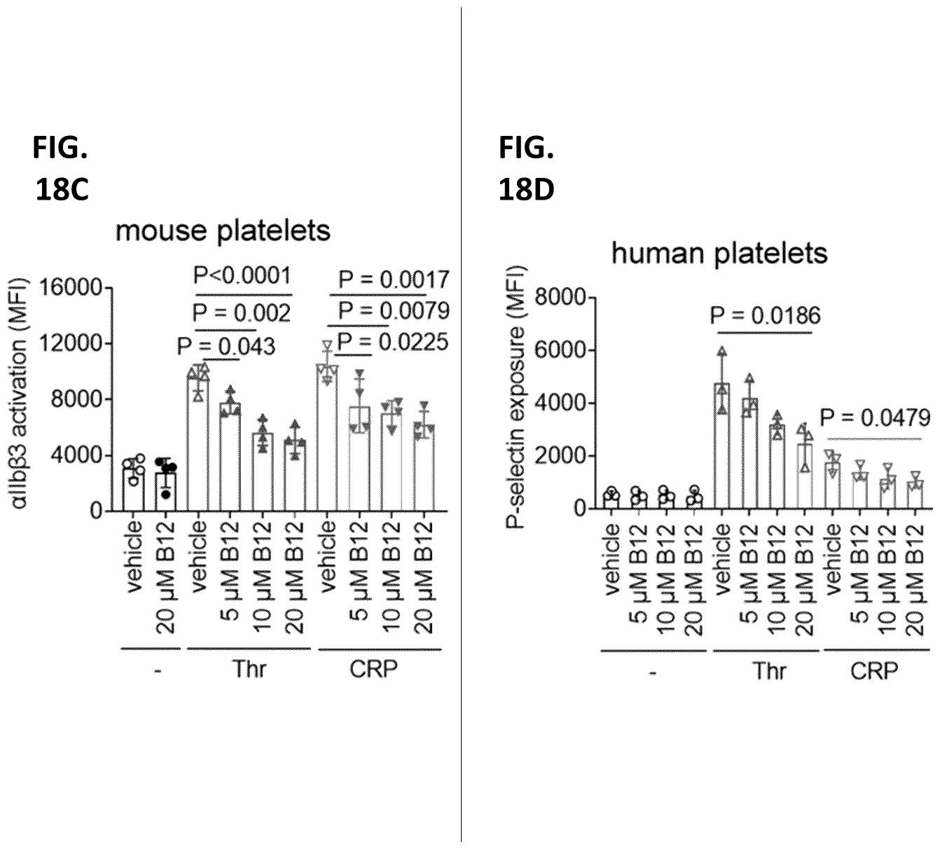

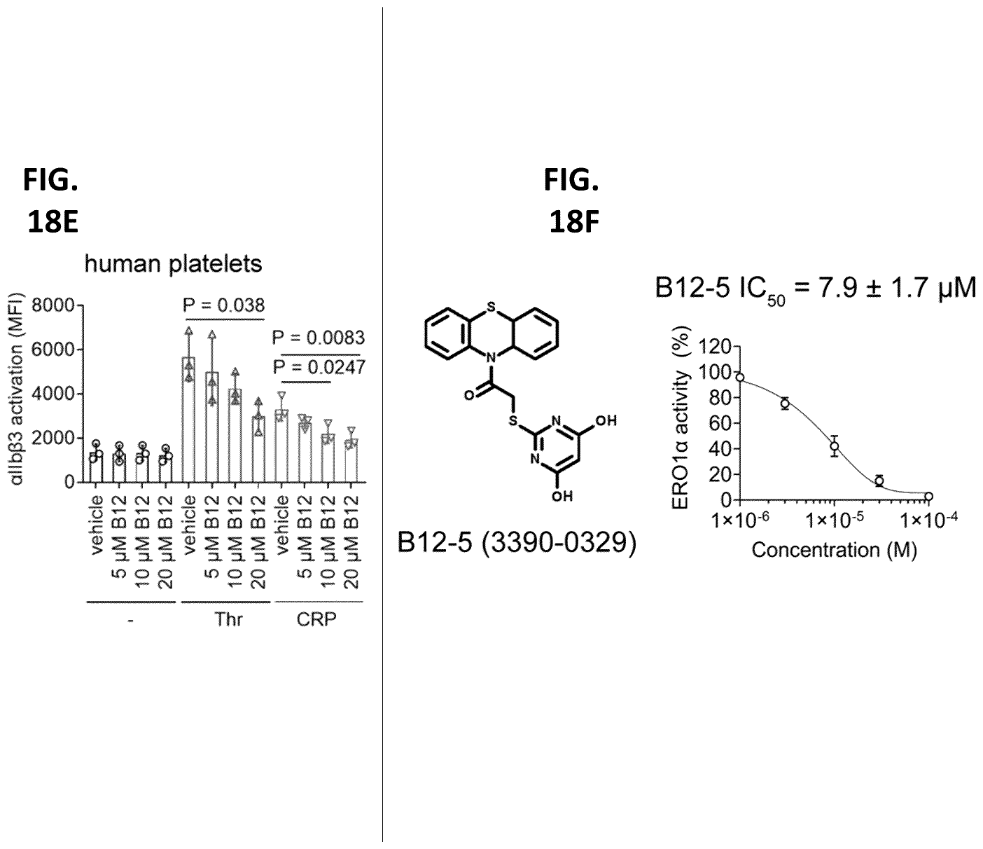

Since ERO1α also interacted with SERCA2 in platelets (FIG. 11C), their direct interaction was examined using recombinant proteins. Pull-down assays showed that ERO1α bound to wtSERCA2 (FIG. 11O). A recent study demonstrated that oxidation or reduction of the intraluminal Cys875-Cys887 disulfide bond controls the ATPase pump activity of SERCA2. ERO1α binding to mutant SERCA2 in which Cys875 and Cys887 residues were replaced with Ser875 and Ser887 (mutant SERCA2 [mSERCA2]-Cys875/887Ser) was significantly reduced compared with wtSERCA2. Nevertheless, N^{α}-(3-maleimidylpropionyl) biocytin-labeled SERCA2 was not detected in platelets (FIG. 16(A-B)). As assessed by mass spectrometry using purified proteins, wtERO1α, but not mERO1α, increased N-ethylmaleimide labeling on the Cys875 residue in SERCA2 (FIG. 11P and FIG. 11Q). The trypsin-digested Cys887-containing peptide, EDNPDFEGVDCAIFESPYPMTMALSVLVTIEMCNALNSLSENQSLLR (SEQ ID NO: 8), was not detected because it is too long. This result suggests that the Cys875 and Cys887 residues in SERCA2 are important for ERO1α binding and implies that ERO1α plays a role in altering the Cys875-Cys887 disulfide bond.

### Novel Small-Molecule ERO1α Inhibitors Recapitulate the Defects in Ero1α-Null Platelets

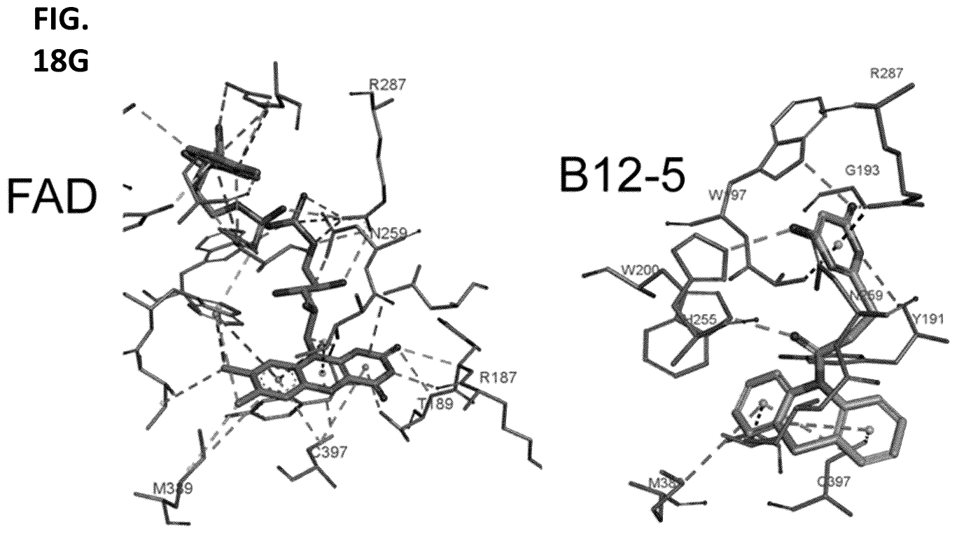

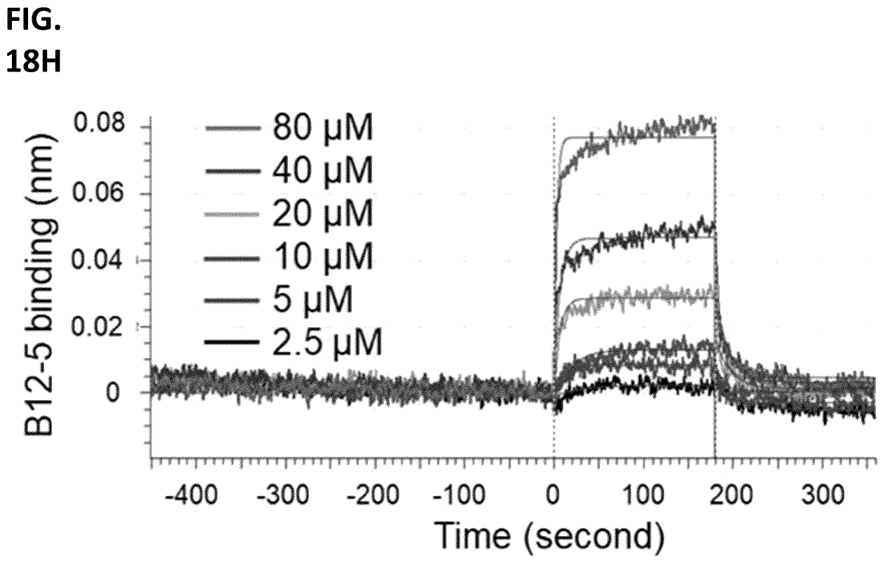

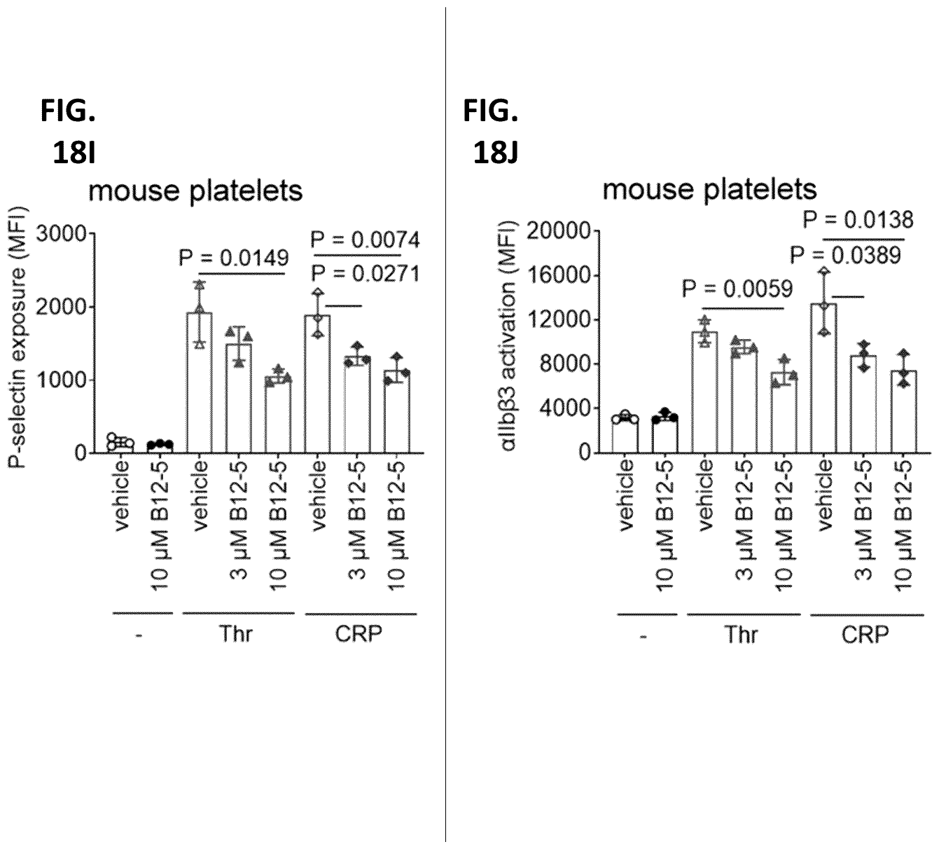

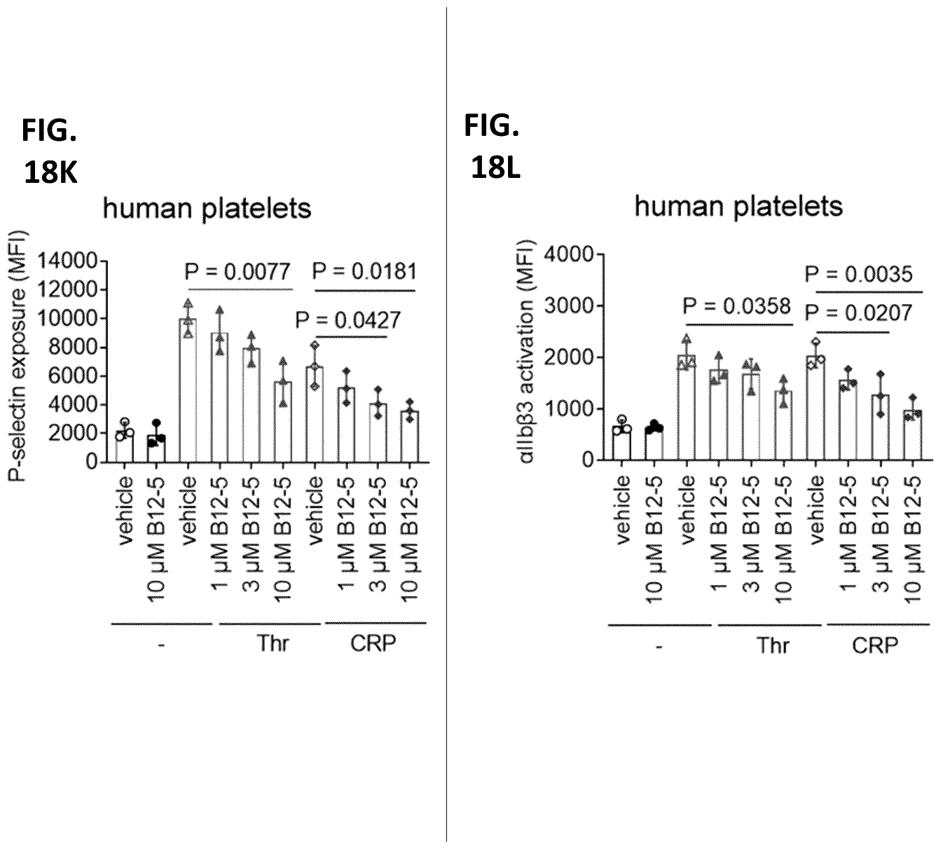

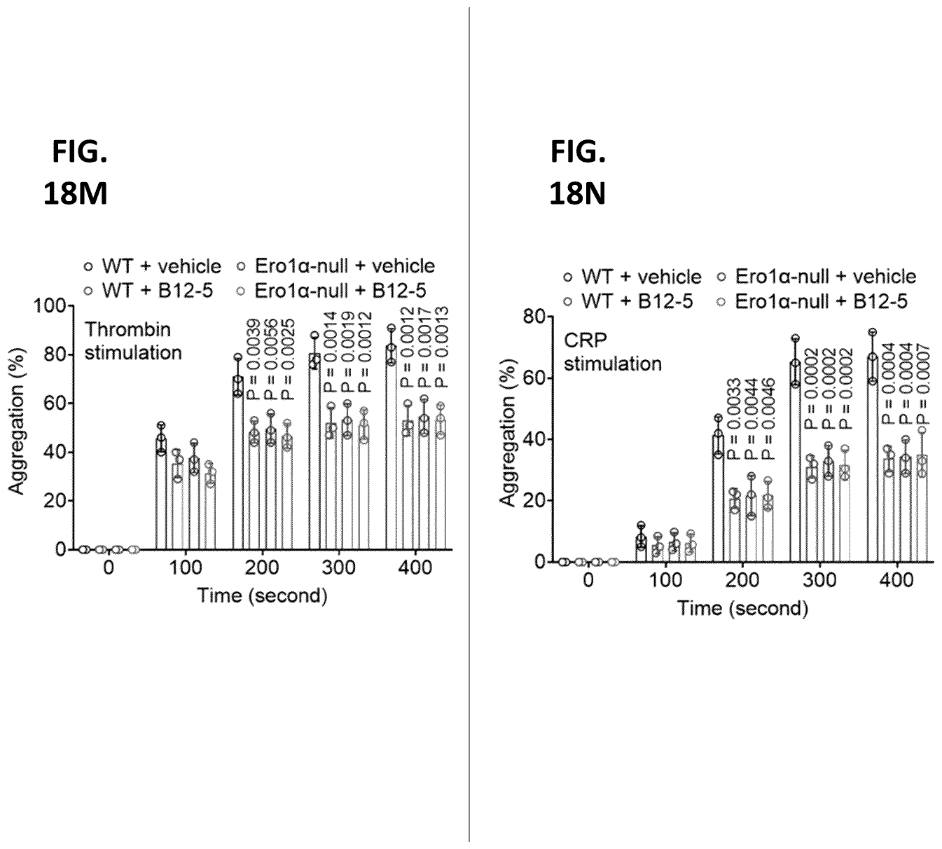

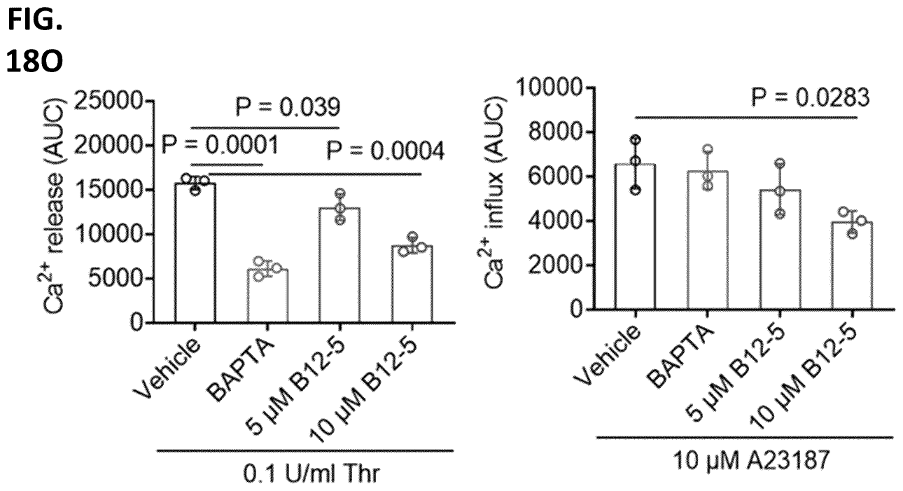

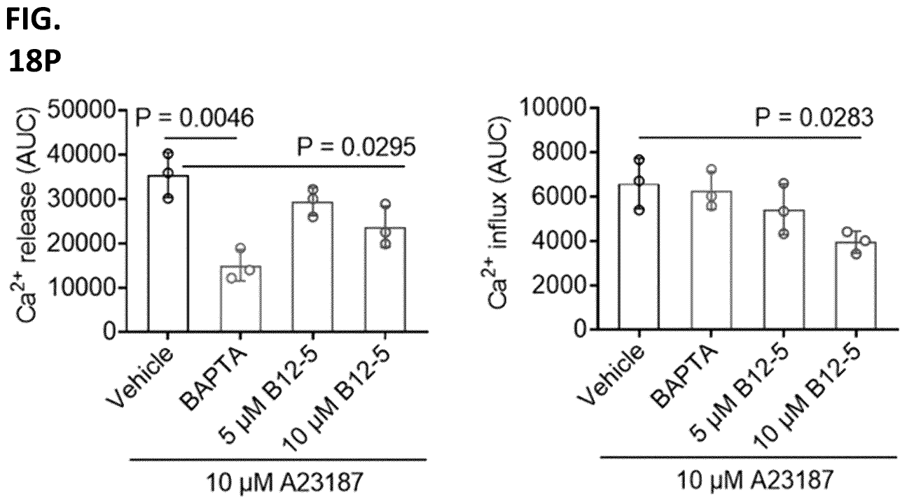

Since deletion of Ero1α significantly inhibits thrombus formation at sites of arteriolar and arterial injury without affecting tail bleeding times (FIG. 2(A-K)), it suggested that targeting ERO1α with small-molecule inhibitors might be an effective strategy to treat thrombotic diseases. Although EN460 has been used in cell-based studies at a concentration of 5 to 50 μM, the compound at 3 to 10 μM significantly inhibited Ero1α-null platelet aggregation and increased the basal Ca²⁺ level in platelets (FIG. 9C and FIG. 14(A-C)), which led to its exclusion from the presently disclosed studies. To identify a novel ERO1α inhibitor, 5800 compounds were screened using an ERO1α activity assay (FIG. 17A and FIG. 17B). The assay demonstrated a Z factor of 0.73, a signal/noise ratio of 14:1, and a coefficient of variance of 4.4%. Compared with 5 μM EN460, 21 compounds exhibited potent inhibitory effects at 20 μM (FIG. 17C). The selected compounds were further tested in assays measuring PDI, antioxidant, and thiol-reacting activities (FIG. 17D through FIG. 17F). B12 was identified as an initial hit with an IC₅₀ value of 12.4 μM and without affecting PDI, antioxidant, and thiol-reacting activities up to 100 μM (FIG. 18A; FIG. 17(A-F)). B12 inhibited P-selectin exposure and αIIbβ3 activation in agonist-activated mouse and human platelets in a concentration-dependent manner (FIG. 18B through FIG. 18E).

22 derivatives of B12 were then tested, from which was identified B12-5, which inhibited ERO1α activity with an IC₅₀ value of 7.9 μM and had a minimal effect on H₂O₂, PDI, and thiol-reacting activities at 100 μM (FIG. 18F; Table S1). However, B12-5 showed a dose-dependent inhibition on the activity of MAO-A (monoamine oxidase A)—another flavin adenine dinucleotide (FAD) coenzyme—by 30% and 51% at 30 and 100 μM, respectively, without affecting its activity at 10 μM. When B12-5 was docked against ERO1α (protein data bank:3AHQ) using Glide within Schrödinger Suite, the phenothiazine head group overlaid well with the tricyclic rings of FAD, forming pi-sulfur interactions with Met389 and Cys397 (FIG. 18G). Also, the tail group of B12-5 followed the same path as the alkyl chain in FAD, which formed hydrogen bonds with His255, Asn259, and Arg287. Using bio-layer interferometry, B12-5 directly bound to ERO1α with a K_{D} value of 12.9±3.8 μM (FIG. 18H). B12-5 at 3 to 10 μM significantly reduced P-selectin exposure and αIIbβ3 activation in mouse and human platelets after stimulation with thrombin or CRP (FIG. 18I through FIG. 18L). Importantly, 10 μM B12-5 significantly mitigated thrombin- or CRP-induced WT platelet aggregation without affecting Ero1α-null platelet aggregation (FIG. 18M and FIG. 18N), suggesting that B12-5 is a specific inhibitor for ERO1α at least up to 10 μM. Also, pretreatment of mouse and human platelets with B12-5 dose dependently inhibited Ca²⁺ release and influx induced by thrombin or A23187 (FIG. 18O through FIG. 18R). Overall, B12-5 was identified as an ERO1α inhibitor that recapitulates the defects observed in Ero1α-null platelets.

Targeting Ero1α Attenuates Arterial Thrombosis and Ischemic Stroke without Affecting Tail Bleeding Times in Mice

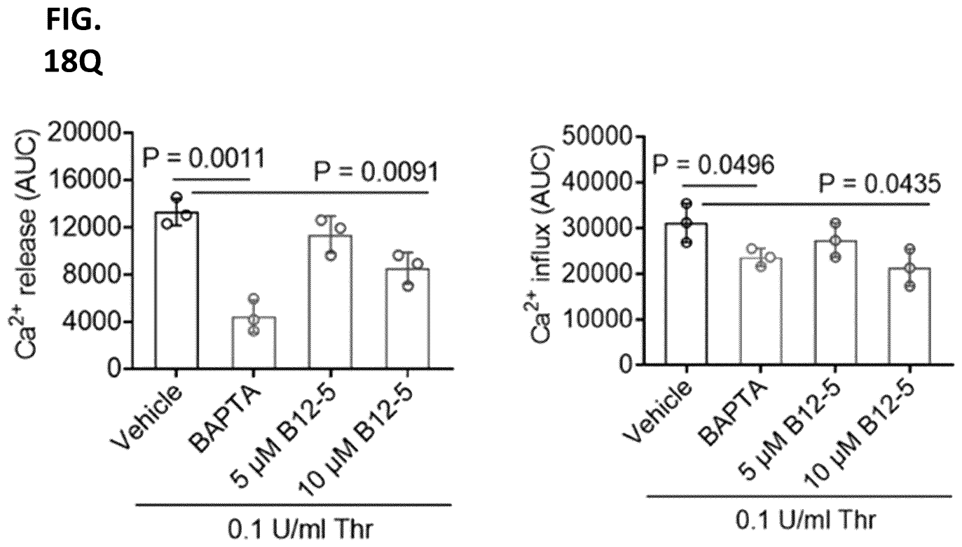

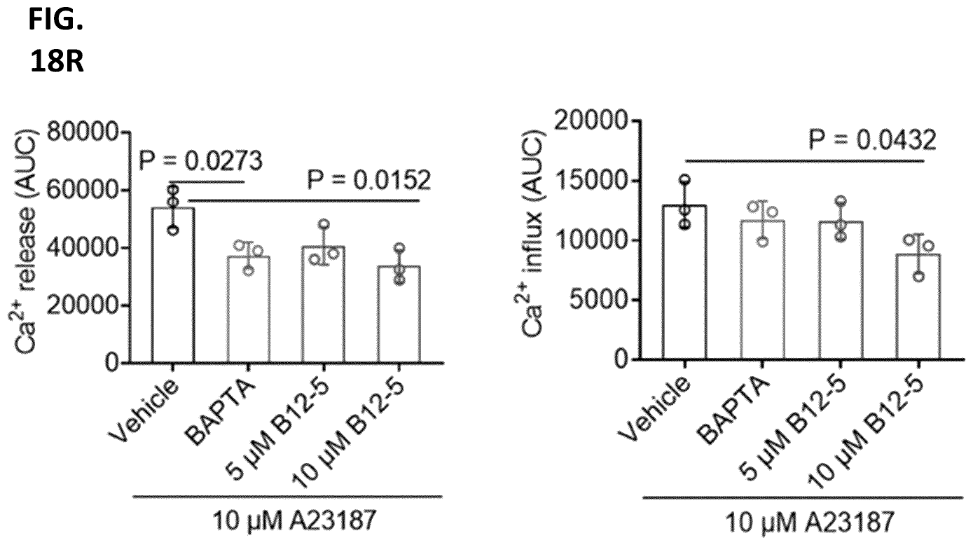

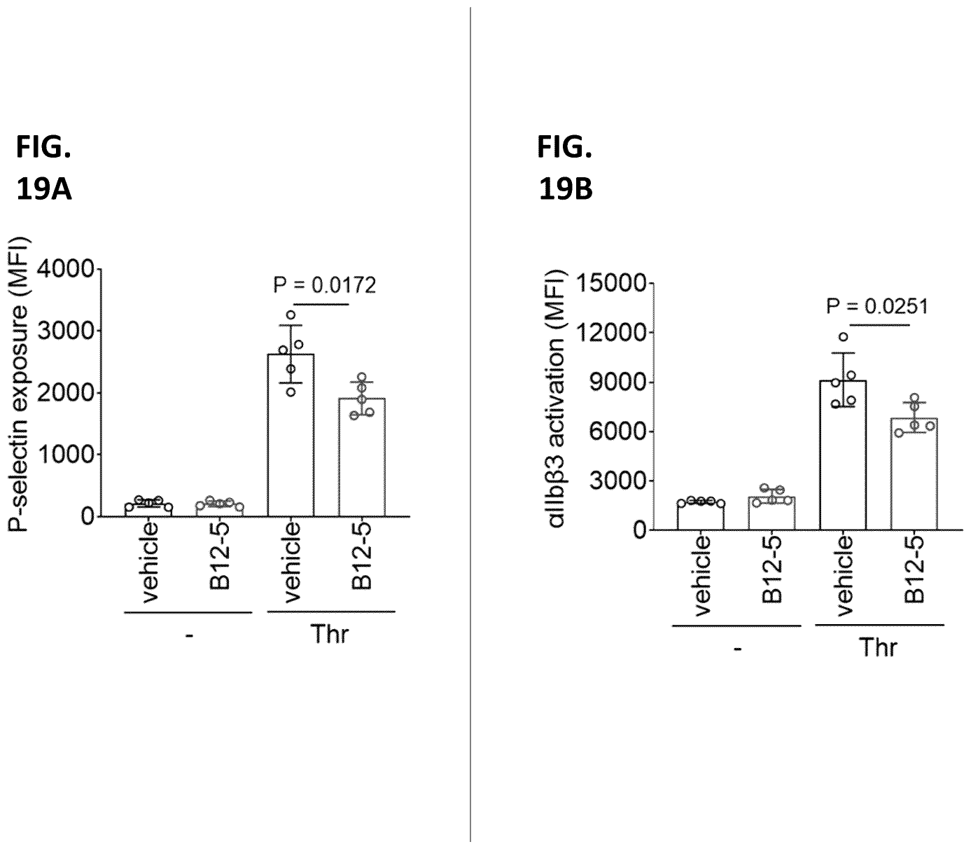

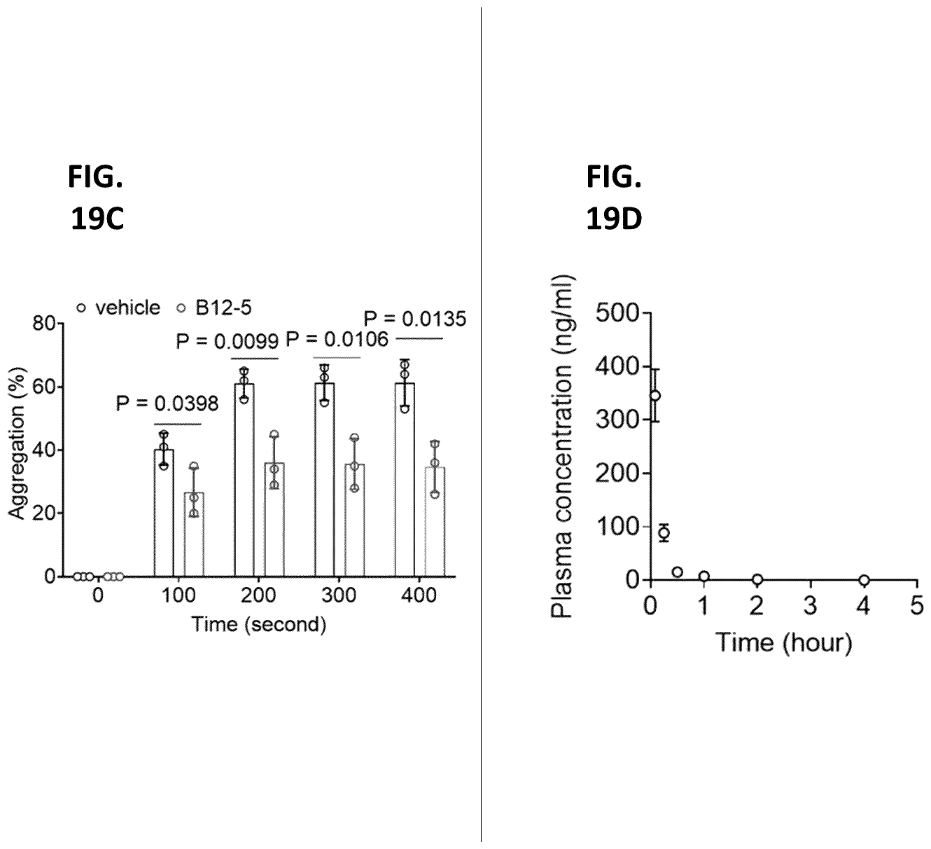

To determine the in vivo efficacy of B12-5 in arterial thrombosis, ex vivo studies were first performed. WT mice were treated with intravenous injection of B112-5, and 1 hour later, blood was drawn to measure the complete blood count and isolate platelets. Compared with the vehicle control, treatment with B12-5 at 5 μg/g BW significantly inhibited P-selectin exposure, αIIbβ3 integrin activation, and aggregation without affecting the complete blood count (FIG. 19A through FIG. 19C; Table 3). A lower dose of B12-5 (2 μg/g BW) did not show inhibitory effects (data not shown), and because of the poor water solubility, B12-5 could not be tested at a higher dose. As measured by liquid chromatography-tandem mass spectrometry, the plasma concentration of B112-5 was 346, 88.7, 15.3, 7.3, and 1.6 ng/mL at 5, 15, 30, 60, and 120 minutes, respectively, after intravenous injection of 5 μg/g BW of B12-5 (FIG. 19D).

TABLE 3
Complete Blood Counts in Vehicle-Treated or B12-5-Treated WT Mice. WT
(C57BL/6) male mice were treated with intravenous injection of vehicle
(20% PEG400 + 3% DMSO) and B12-5 (5 μg/g BW). One hour later,
blood was drawn, and cells were counted using HEMAVET 950 (Drew Scientific).

| | WBC, | NE, | LY, | MO, | RBC, | PLT, | MPV, |
|---|---|---|---|---|---|---|---|
| | 10³/μL | 10³/μL | 10³/μL | 10³/μL | 10⁶/μL | 10³/μL | fL |
| Vehicle | 5.6 ± 1.0 | 0.7 ± 0.2 | 4.5 ± 0.8 | 0.2 ± 0.1 | 10.1 ± 1.5 | 954 ± 156 | 4.6 ± 0.3 |
| B12-5 | 5.4 ± 1.1 | 0.7 ± 0.2 | 4.2 ± 0.8 | 0.2 ± 0.1 | 9.9 ± 1.2 | 847 ± 114 | 4.6 ± 0.4 |

The data represent the mean ± SD (n = 6 mice [8-12 wk old] per group). BW indicates body weight; DMSO, dimethyl sulfoxide; LY, lymphocyte; MO, monocyte; MPV, mean platelet volume; NE, neutrophil; PLT, platelet; RBC, red blood cell; WBC, white blood cell; and WT, wild type.

As assessed by intravital microscopy, intravenous injection of B12-5 (5 μg/g BW) into WT mice significantly inhibited platelet thrombus formation but did not affect fibrin generation at the site of laser-induced cremaster arteriolar injury (FIG. 19E and FIG. 19F). Consistent with a previous report, injection of eptifibatide (5 μg/g BW)—an αIIbβ3 integrin antagonist—inhibited platelet thrombus formation without reducing fibrin generation. To see the antithrombotic effect of B12-5 in a large artery, the compound was tested in a mouse model of FeCl₃-induced carotid arterial thrombosis. Compared with the vehicle control, both B112-5 and eptifibatide significantly prolonged the time to occlusion (FIG. 5G). However, treatment with eptifibatide markedly prolonged tail bleeding times and increased blood loss in mice, whereas B12-5 did not affect (FIG. 19H and FIG. 19I).

Ischemic stroke results from thrombus-mediated obstruction of cerebral blood flow and subsequent reperfusion injury in which platelets and immune cells contribute to disease pathology. To determine the role of ERO1α in ischemic stroke, megakaryocyte-specific Ero1α CKO and global KO mice were tested in a model of transient middle cerebral artery occlusion (for 45 minutes) and subsequent reperfusion (23 hours). Compared with WT control, Ero1α KO mice exhibited a significant reduction in infarct volume after transient middle cerebral artery occlusion and improvement in neurological deficits as assessed by the Bederson score and Grip test (FIG. 19J through FIG. 19M). Megakaryocyte-specific deletion of Ero1α minimally affected infarct volume and neurological deficits. A single intravenous injection of 5 μg/g BW of B12-5 into WT (C57BL/6) mice immediately after transient middle cerebral artery occlusion significantly reduced infarct volume and ameliorated neurological deficits compared with vehicle control (FIG. 19N through FIG. 19Q). These results suggest that ERO1α can be a pharmacological target for treating ischemic stroke.

### DISCUSSION

Platelet-released PDI binds to the platelet surface, enhancing the ligand-binding function of platelet surface molecules and promoting thrombogenesis. As disclosed herein, the following hypothesis was tested: that platelet ERO1α plays a role in arterial thrombosis by regulating extracellular PDI activity. Using a wide range of in vitro and in vivo studies with Ero1α CKO and KO mice, novel blocking antibodies, and small-molecule inhibitors, it was shown that the hypothesis was incorrect. Instead, it was demonstrated that ERO1α is neither released from platelets nor detected on the platelet surface and that deletion or inhibition of Ero1α in mice significantly reduces platelet thrombus formation in arterial thrombosis and infarct volume in ischemic stroke without affecting tail bleeding times. Intracellular ERO1α contributes to Ca²⁺ store content and promotes Ca²⁺ mobilization and platelet activation and aggregation in a PDI-independent manner. Mechanistically, ERO1α directly interacts with STIM1 and SERCA2, oxidizes an allosteric disulfide bond, and regulates their function. Ero1α, unlike Pdi, is dispensable for mouse survival. Treatment of mice with blocking anti-PDI antibodies prolongs tail bleeding times in mice. Therefore, the presently disclosed results suggest that compared with PDI, ERO1α can be a more attractive target for developing an antithrombotic agent.

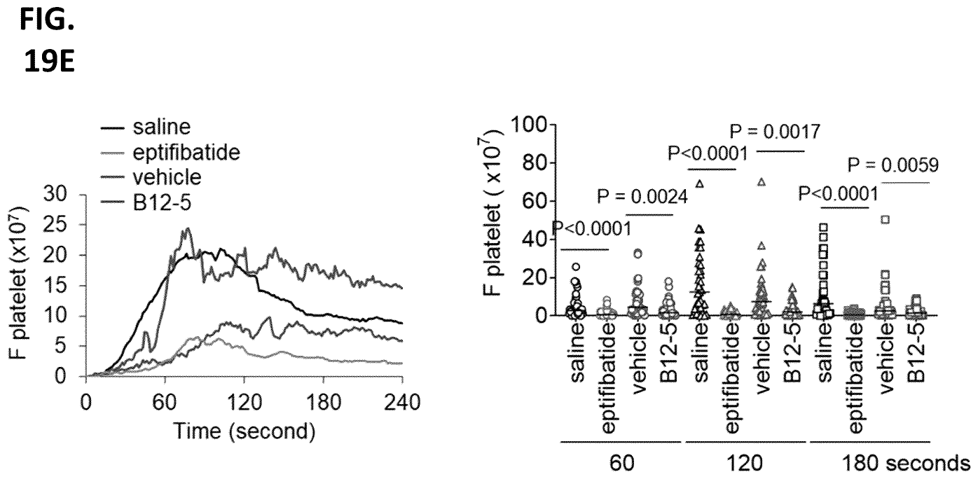

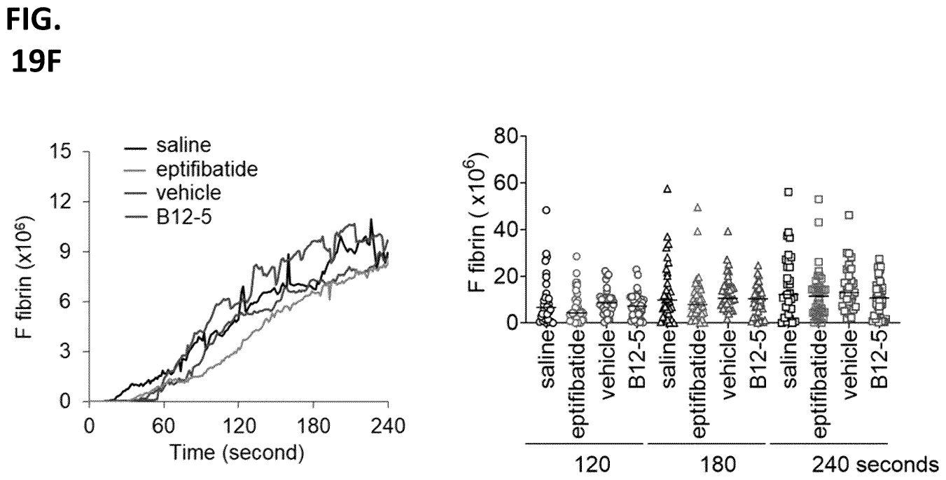

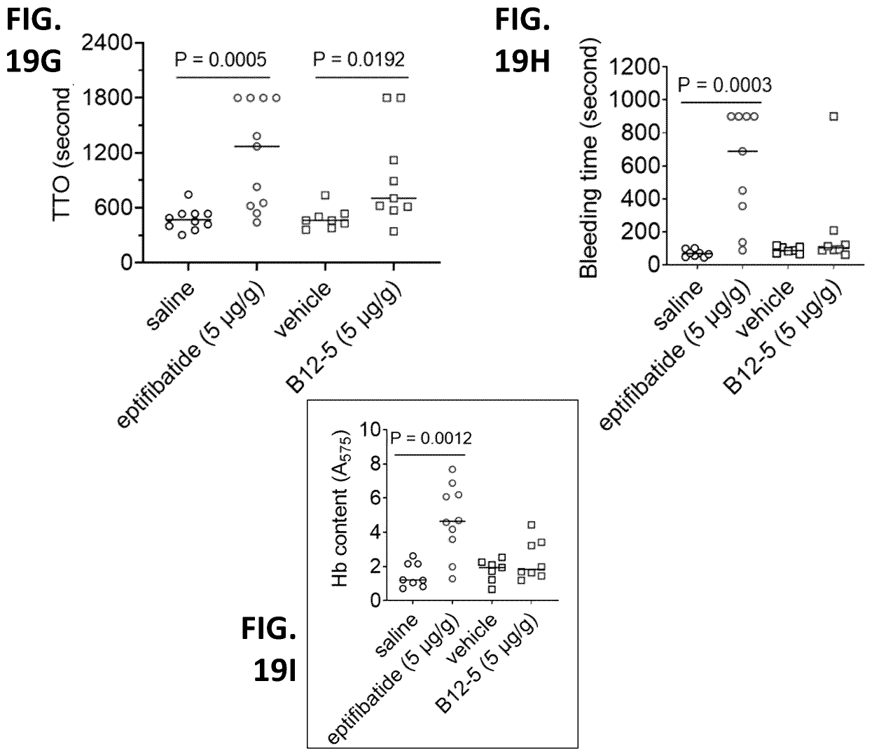

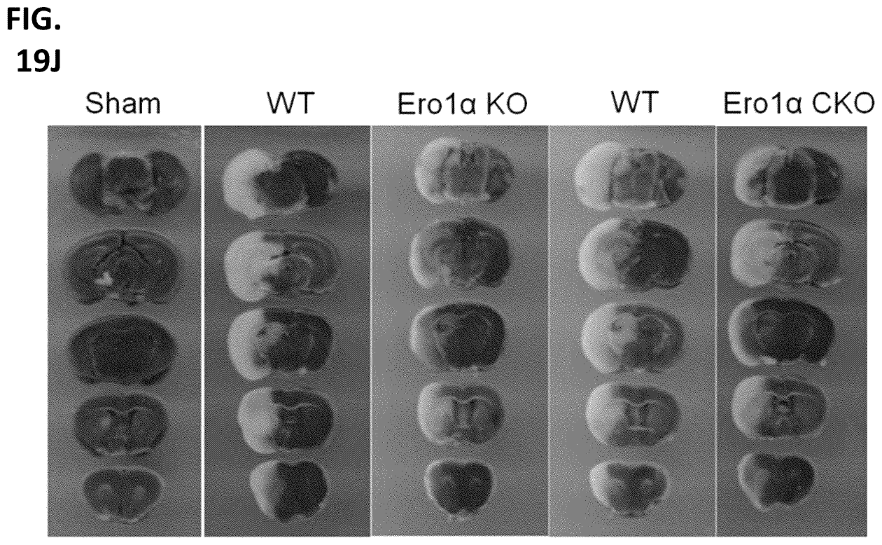

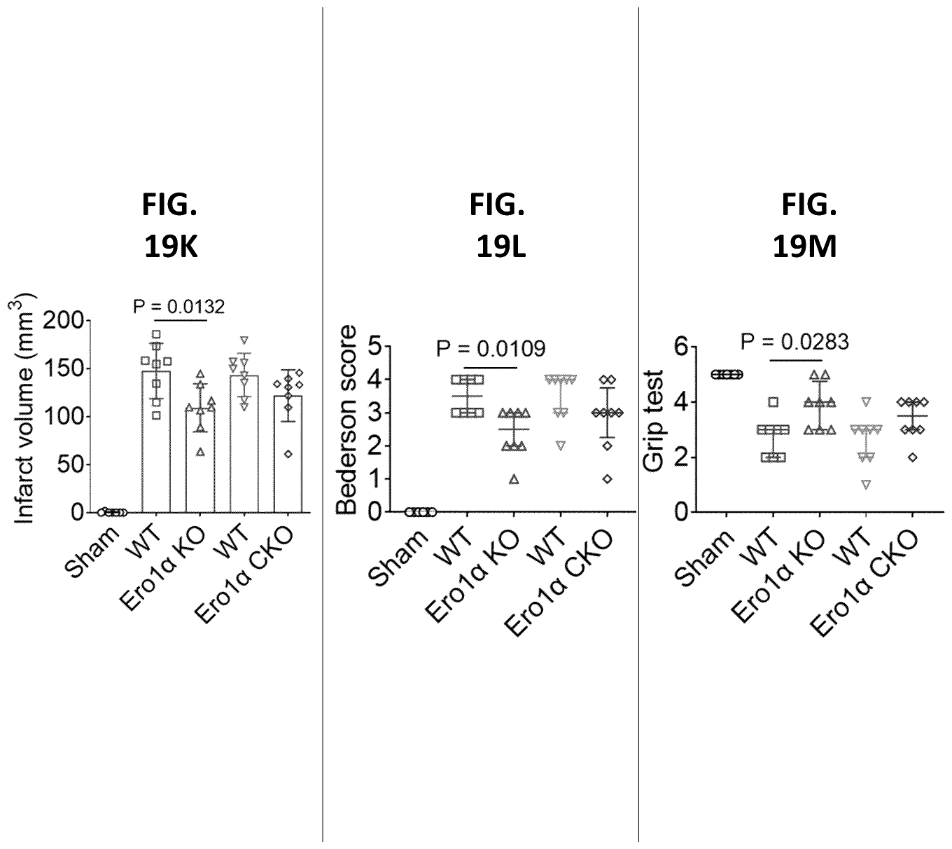

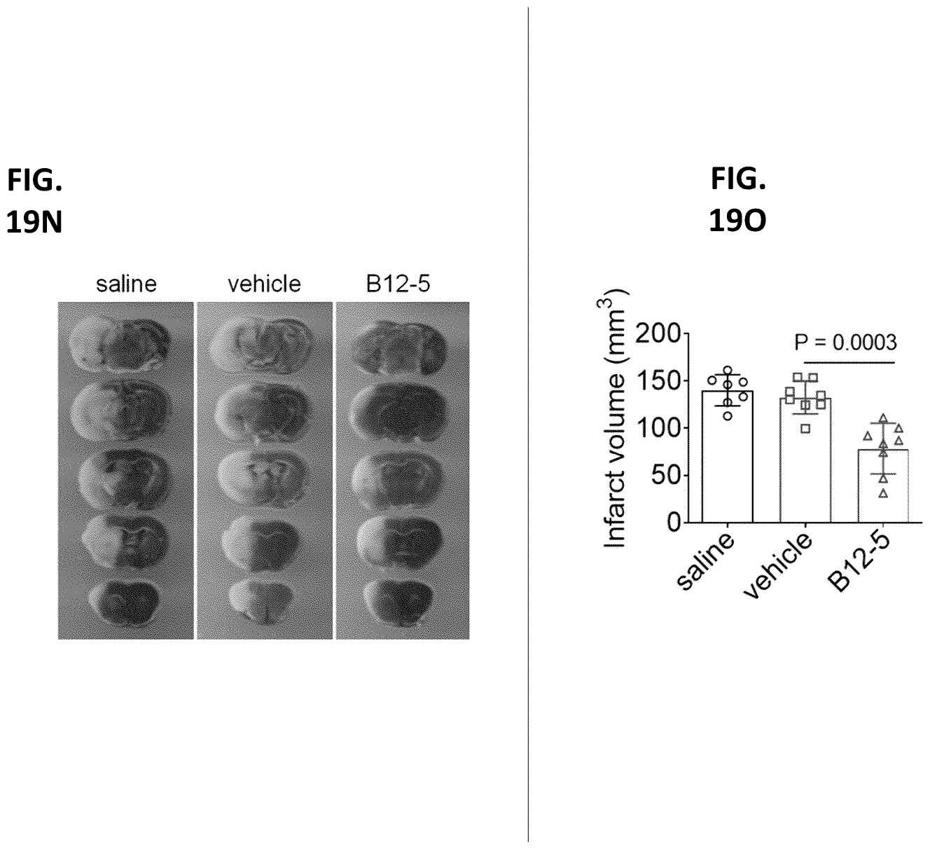

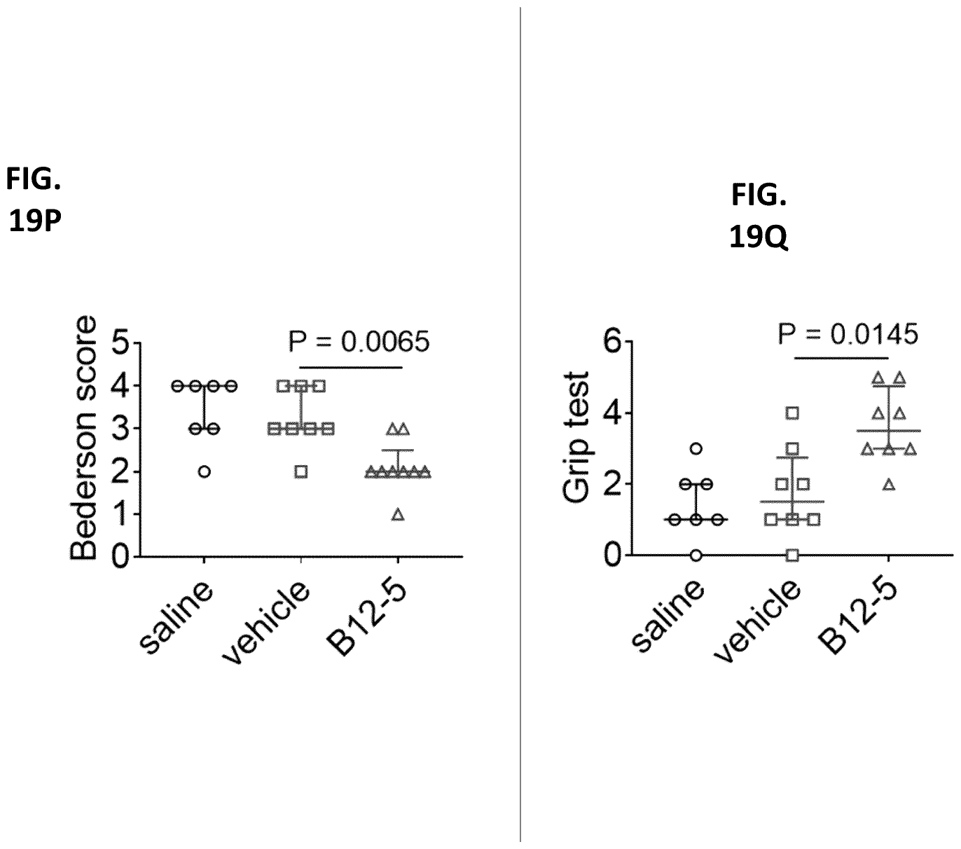

Pdi is detected at sites of laser-induced arteriolar injury and within developing platelet thrombi in mice. The kinetics of Pdi accumulation correlates with that of platelet accumulation, whereas the kinetics of Ero1α accumulation does not (FIG. 2B and FIG. 2C). Further, a similar level of extracellular Ero1α is detected at the site of laser injury in WT control and Ero1α CKO mice, but its level is negligible in Ero1α KO mice (FIG. 20). Although ERO1α may be present in circulation, plasma Ero1α was not detected by immunoblotting with a detection limit of 1 ng/mL (FIG. 21). These results suggest that while extracellular Pdi is derived from both laser-damaged cells and aggregating platelets, extracellular Ero1α is from laser-damaged cells, but not from platelets. Furthermore, inhibition of extracellular Pdi with blocking anti-PDI antibodies significantly reduces arteriolar thrombogenesis in mice. In contrast, treatment of mice with 15E9 does not affect platelet thrombus formation and fibrin generation (FIG. 7K and FIG. 7L). Overall, the findings of the present disclosure suggest that Ero1α detected at sites of vascular injury has a minimal role in thrombogenesis.

It is reported that platelet-released or surface ERO1α regulates αIIbβ3 integrin function and promotes platelet aggregation through PDI. However, the in vitro studies used high concentrations of recombinant PDI and ERO1α, which are >1000× greater than those found under healthy and disease conditions. Furthermore, the authors did not prove whether and how ERO1α is secreted from activated platelets. Using flow cytometry and immunogold electron microscopy, it was demonstrated that ERO1α is not detected on the platelet surface or released from activated platelets but localizes exclusively in the DTS (FIG. 5(A-C); FIG. 11G). Consistently, treatment with 15E9 does not affect platelet activation and aggregation (FIG. 10(A-E)). While the aggregation defect in Pdi-null platelets was restored to the WT level in the presence of recombinant wtPDI, such a rescue effect was not observed in Ero1α-null platelets by exogenously added wtERO1α or wtPDI (FIG. 7F). Moreover, unlike PDI, platelet ERO1α is crucial for platelet activation, Ca²⁺ store content, and SOCE (FIG. 7G, FIG. 7H, and FIG. 11D through FIG. 11F). These results suggest that intracellular ERO1α promotes platelet activation by regulating Ca²⁺ signaling in a PDI-independent manner.

Previously reported loss of platelet STIM1 contributes to Ca²⁺ store content and impairs Ca²⁺ mobilization induced by thapsigargin. This study also showed that deletion of hematopoietic cell STIM1 in mice prolongs the time to occlusion in FeCl₃-induced mesenteric arteriolar thrombosis. Similar results were obtained from Ero1α CKO mice, supporting the finding that platelet Ero1α positively regulates STIM1 function. Growing evidence indicates that a Cys49-Cys56 disulfide bond near the EF-hand 1 domain in STIM1 has an allosteric function. S-nitrosylation at the Cys49 and Cys56 residues suppresses Ca²⁺ depletion-dependent oligomerization of the endoplasmic reticulum luminal domain and inhibits SOCE in cardiomyocytes and HEK293 (human embryonic kidney 293) cells. Furthermore, ERp57 cleaves the Cys49-Cys56 disulfide bond, impairing STIM1-mediated SOCE. As shown herein, platelet ERO1α interacts with STIM1 through the Cys49 and Cys56 residues and forms the Cys49-Cys56 disulfide bond during cell activation, thereby contributing to Ca²⁺ store content in the DTS and promoting Ca²⁺ mobilization. Furthermore, loss of STIM1 significantly reduces SOCE in neutrophils stimulated with a Ca²⁺ ionophore. This study may explain how loss of Ero1α impairs SOCE in A23187-stimulated platelets. Overall, the results of the present disclosure provide the first evidence that ERO1α-mediated oxidation of the allosteric Cys49-Cys56 disulfide bond positively regulates STIM1 function.

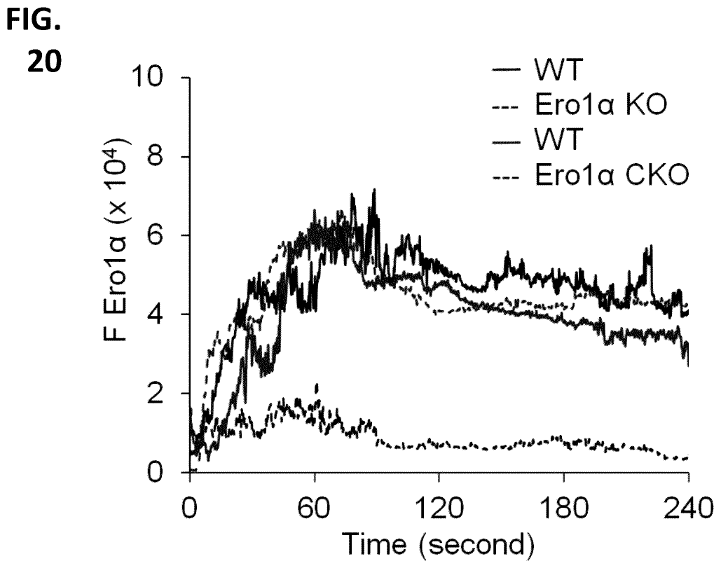

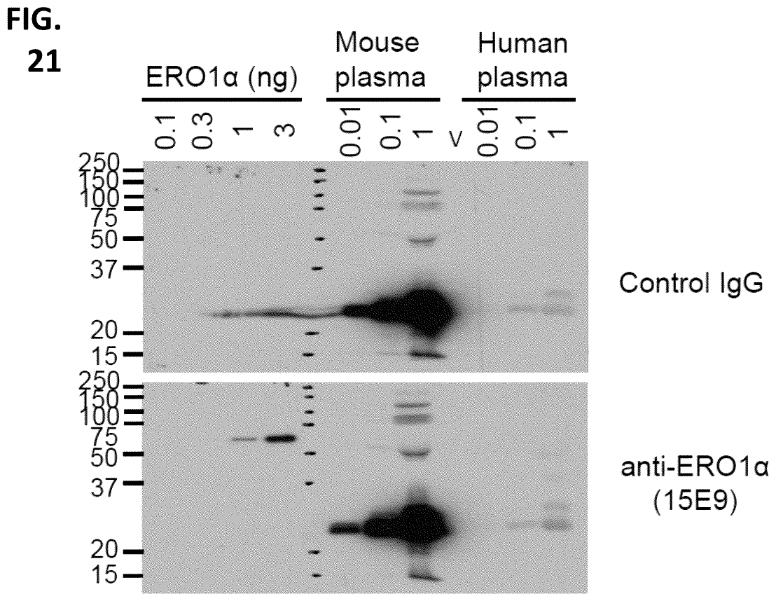

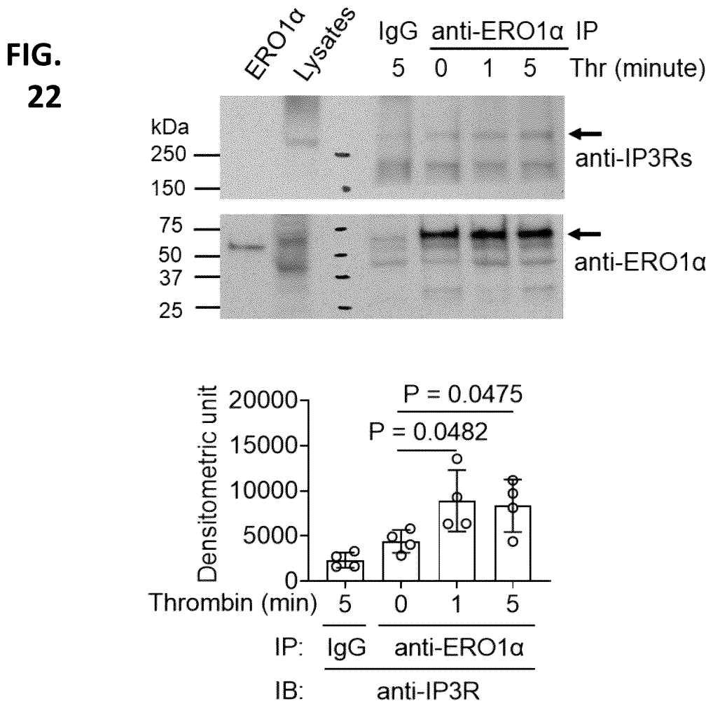

STIM1-null platelets showed defects in platelet activation and aggregation induced by CRP but not ADP or thrombin. However, Ero1α-null platelets exhibit the functional defect after stimulation with CRP and thrombin (FIG. 7B, FIG. 7C, FIG. 7G, and FIG. 7H). This result suggests that the regulatory function of ERO1α is not limited to STIM1. It is reported that the pump activity of SERCA2 is inhibited by ERp57-mediated oxidation of a Cys875-Cys887 disulfide bond in the intraluminal loop 4 and is enhanced by ERdj5-mediated cleavage of the disulfide bond. ERO1α interacts with SERCA2 and that their binding depends on Cys875 and Cys887 residues in SERCA2 (FIG. 11C and FIG. 11O). Surprisingly, mass spectrometric analysis shows increased N-ethylmaleimide labeling on Cys875 in the presence of wtERO1α (FIG. 11P and FIG. 11Q). Since Cys875 and Cys887 residues are already oxidized in recombinant SERCA2 and ERO1α acts as an oxidase but not a reductase, this may be due to a transient disulfide bond between ERO1α and the Cys875 or Cys887 residue in SERCA2. In addition, mass spectrometry combined with immunoprecipitation shows that ERO1α may interact with IP3Rs (inositol 1,4,5-trisphosphate receptors) that induce Ca²⁺ release after thrombin stimulation. It is reported that ERO1α stimulates IP3Rs during macrophage apoptosis and that IP3R function is regulated by thiol modification. Although increased ERO1α-IP3Rs in activated platelets was observed (FIG. 22), the mechanism was not immediately identified due to the difficulty of expressing this high-molecular-weight protein (320 kDa). Further studies are required to investigate whether ERO1α regulates the function of IP3Rs after thrombin stimulation.

Studies suggest that EN460 inhibits not only ERO1α (IC₅₀, 1.9 μM) but also other FAD-containing enzymes, including MAO-A (IC₅₀, 7.9 μM) and MAO-B (IC₅₀, 30.6 μM), and has thiol-reacting activity. Furthermore, the compound at 3 μM completely blocked agonist-induced Ero1α-null platelet aggregation (FIG. 9C). In contrast, B12-5 did not show an additional effect on Ero1α-null platelet aggregation up to 10 μM (FIG. 4M and FIG. 18N), indicating a specific inhibitory effect on Ero1α. Although the high concentration (100 μM) of B12-5 did not affect antioxidant, PDI, and thiol-reacting activities, it inhibited MAO-A activity (Table 4), which may affect platelet aggregation. In addition, since there is a high sequence homology (around 65%) in the FAD binding pocket of ERO1α and ERO1β, the B12-5 compound is likely to inhibit the activity of both ERO1 isoforms. Future studies will identify additional selective and potent ERO1α inhibitors.

TABLE 4
Identified compounds and characteristics.
General structure for B12 compound in Table 4: 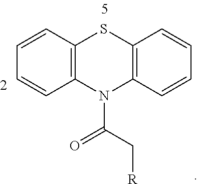
While B12-8* and B12-13* have a different head structure:

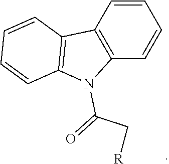

| | | | | ERO1α | PDI | H₂O₂ | GSH | MAO-A |
|---|---|---|---|---|---|---|---|---|
| | | | | inhibition | inhibition | inhibition | inhibition | inhibition |
| | | | | at | at | at | at | at |
| | | | | 20 μM | 100 μM | 100 μM | 100 μM | 100 μM |
| Name | R | 2 | 5 | (%) | (%) | (%) | (%) | (%) |
| B12 (D585- 0145) | 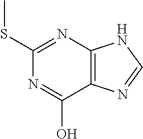 | H | S | 72.9 ± 12 | 4.8 ± 1 | 4.7 ± 1 | 5.1 ± 2 | 25.3 ± 5 |
| B12-1 (2683- 0226) | 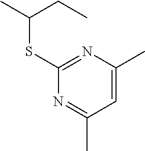 | H | S | 60.6 ± 21 | 19.1 ± 4 | 1.3 ± 1 | 39.1 ± 10 | 23.8 ± 5 |
| B12-2 (3219- 0020) | 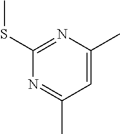 | H | S | 90.0 ± 23 | 17.3 ± 4 | 0.4 ± 0.1 | 26.8 ± 7 | 29.8 ± 5 |
| B12- 3 (3389- 0112) | 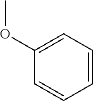 | H | S | 57.0 ± 19 | 10.2 ± 3 | 2.1 ± 1 | 22.3 ± 7 | 24.8 ± 4 |
| B12- 4 (3390- 0150) | 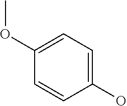 | H | S | 74.0 ± 17 | 13.2 ± 3 | 6.0 ± 2 | 27.2 ± 7 | 34.8 ± 4 |
| B12- 5 (3390- 0329) | 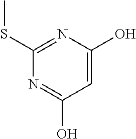 | H | S | 90.2 ± 2 1 | 8.2 ± 2 | 6.6 ± 2 | 9.7 ± 2 | 50.7 ± 9 |
| B12-6 (3653- 0102) | 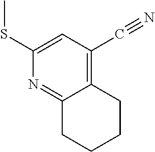 | H | S | 87.6 ± 17 | 20.2 ± 4 | 1.7 ± 1 | 30.1 ± 10 | 37.7 ± 8 |
| B12- 7 (4327- 3206) | 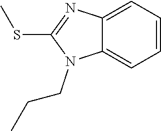 | H | S | 75.5 ± 20 | 30.3 ± 8 | 1.9 ± 1 | 33.3 ± 10 | 39.1 ± 8 |
| B12- 8* (4477- 0954) | 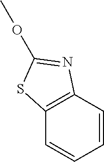 | H | C | 48.6 ± 10 | 8.6 ± 3 | 0.3 ± 0.1 | 13.6 ± 3 | 22.3 ± 5 |
| B12- 9 (4477- 2496) | 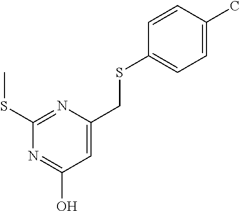 [SMILES: CSc1nc(O)cc(CSc2ccc(C)cc2)n1] | H | S | 87.5 ± 11 | 20.4 ± 7 | 1.7 ± 0.4 | 35.4 ± 9 | 22.9 ± 6 |
| B12- 10 (4934- 0242) | 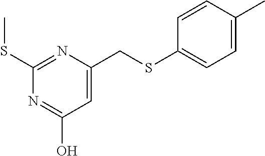 [SMILES: CSc1nc(O)cc(CSc2ccc(C)cc2)n1] | H | S | 86.7 ± 7.2 | 18.6 ± 4 | 7.8 ± 2 | 35.6 ± 5 | 31.1 ± 6 |
| B12- 11 (5227- 4869) | 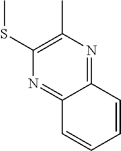 | H | S | 82.3 ± 20 | 23.1 ± 5 | 2.3 ± 1 | 33.5 ± 4 | 28.1 ± 5 |
| B12- 12 (5645- 0130) | 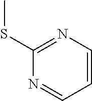 | Cl | S | 71.8 ± 10 | 20.1 ± 5 | 1.5 ± 1 | 24.1 ± 7 | 38.1 ± 7 |
| B12- 13* (6186- 3409) | 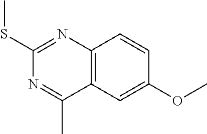 | C | H | 53.7 ± 9 | 19.1 ± 4 | 4.2 ± 1 | 33.1 ± 4 | 36.7 ± 8 |
| B12- 14 (8009- 4919) | 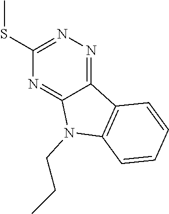 [SMILES: CCCn1c2ccccc2c2nnc(SC)nc21] | H | S | 59.0 ± 6 | 21.4 ± 6 | 3.4 ± 1 | 31.9 ± 5 | 28.3 ± 5 |
| B12- 15 (8010- 8466) | 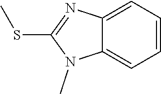 | H | S | 100.8 ± 22 | 21.3 ± 7 | 0.2 ± 0.1 | 34.7 ± 7 | 34.3 ± 6 |
| B12- 16 (8011- 7594) | 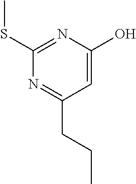 | H | S | 11.5 ± 3 | 11.9 ± 3 | 80.0 ± 18 | 21.9 ± 6 | 34.3 ± 6 |
| B12- 17 (8012- 8310) | 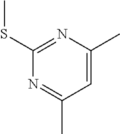 | Cl | S | 61.8 ± 13 | 10.2 ± 3 | −1.2 ± 0.2 | 20.5 ± 3 | 37.1 ± 8 |
| B12- 18 (8018- 4648) | 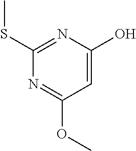 | H | S | 83.9 ± 16 | 12.4 ± 5 | 0.2 ± 0.1 | 25.9 ± 5 | 37.5 ± 7 |
| B12- 19 (Y020- 0073) | 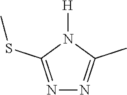 | H | S | 73.5 ± 16 | 6.2 ± 2 | −2.7 ± 1 | 16.4 ± 3 | 28.8 ± 6 |
| B12- 20 (Y020- 0074) | 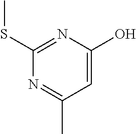 | H | S | 82.3 ± 15 | 6.7 ± 2 | 2.3 ± 1 | 10.2 ± 2 | 50.2 ± 7 |
| B12- 21 (Y020- 0238) | 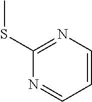 | H | S | 70.3 ± 8 | 2.6 ± 1 | 2.2 ± 0.4 | 3.6 ± 1 | 37.8 ± 6 |
| B12- 22 (Y020- 4978) | 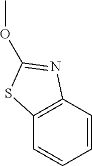 | H | S | 60.5 ± 8 | 38.5 ± 8 | 4.9 ± 1 | 62.5 ± 7 | 28.8 ± 6 |

A single intravenous injection of B112-5 or eptifibatide into mice was found to inhibit platelet thrombus formation without affecting fibrin generation at the site of laser-induced cremaster arteriolar injury (FIG. 19E and FIG. 19F). The presently disclosed finding that the ex vivo and in vivo effects of B12-5 on platelet activation and aggregation are maintained at least for 1 hour after injection (FIG. 19(A-Q)), indicating that B12-5 gets into circulating platelets and exhibits the effect. In addition to immune cells, platelets play a role in the pathology of ischemic stroke. Despite an understanding of disease pathology, ischemic stroke remains a major cause of death and disability in the United States, and there is no effective therapy. As disclosed herein, deletion of Ero1α and a single intravenous injection of B112-5 after transient middle cerebral artery occlusion significantly reduces infarct volume and improves neurological deficits in a mouse model of ischemic stroke, whereas megakaryocyte-specific deletion of Ero1α has a minimal effect (FIG. 19J through FIG. 19Q). These results imply that brain and intravascular cell Ero1α may contribute to the pathogenesis of ischemic stroke. Further studies are required to determine the role of other cellular ERO1α in ischemic brain injury.

Overall, platelet ERO1α was identified as a novel regulator of Ca²⁺ signaling during platelet activation. The present disclosure demonstrates proof of principle that targeting ERO1α can be a therapeutic strategy for preventing or treating thrombotic diseases, such as atherothrombosis and ischemic stroke.

What is claimed is:

1. A method of inhibiting ERO1α in a subject having a thrombotic disease, the method comprising administering to the subject at least one ERO1α inhibitor.

2. The method of claim 1, wherein the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.

3. The method of claim 1, wherein administering the at least one ERO1α inhibitor reduces at least one of:
platelet Ca²⁺ release,
ERO1α interaction with STIM1,
ERO1α interaction with SERCA2, and
Cys49-Cys56 disulfide bond reoxidation in STIM1.

4. The method of claim 1, wherein administering the at least one ERO1α inhibitor does not significantly affect at least one of:
initial platelet adhesion,
fibrin generation,
blood loss,
bleeding times, and
hemostasis in the subject.

5. The method of claim 1, wherein the at least one ERO1α inhibitor is selected from B12, B12-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B112-22.

6. The method of claim 5, wherein the at least one ERO1α inhibitor is B12-5.

7. The method of claim 1, wherein the at least one ERO1α inhibitor does not comprise an anti-ERO1α antibody.

8. A method of reducing platelet thrombus formation in subject having a thrombotic disease, the method comprising administering to the subject at least one ERO1α inhibitor.

9. The method of claim 8, wherein the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.

10. The method of claim 8, wherein administering the at least one ERO1α inhibitor reduces at least one of:
platelet Ca²⁺ release,
ERO1α interaction with STIM1,
ERO1α interaction with SERCA2, and
Cys49-Cys56 disulfide bond reoxidation in STIM1.

11. The method of claim 8, wherein administering the at least one ERO1α inhibitor does not significantly affect at least one of:
initial platelet adhesion,
fibrin generation,
blood loss,
bleeding times, and
hemostasis in the subject.

12. The method of claim 8, wherein the at least one ERO1α inhibitor is selected from B12, B12-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B12-22.

13. The method of claim 12, wherein the at least one ERO1α inhibitor is B12-5.

14. The method of claim 8, wherein the at least one ERO1α inhibitor does not comprise an anti-ERO1α antibody.

15. A composition for treating a thrombotic disease in a subject in need thereof, the composition comprising at least one ERO1α inhibitor.

16. The composition of claim 15, wherein the at least one ERO1α inhibitor is selected from B12, B12-1, B12-2, B12-3, B12-4, B12-5, B12-6, B12-7, B12-8, B12-9, B12-10, B12-11, B12-12, B12-13, B12-14, B12-15, B12-16, B12-17, B12-18, B12-19, B12-20, B12-21, and B12-22.

17. The composition of claim 16, wherein the at least one ERO1α inhibitor is B12-5.

18. The composition of claim 15, wherein the at least one ERO1α inhibitor does not comprise an anti-ERO1α antibody.

19. The composition of claim 15, wherein the at least one ERO1α inhibitor has an IC50 value of less than about 13 μM.

20. The composition of claim 15, wherein the thrombotic disease is selected from atherothrombosis, ischemic stroke, coronary artery disease, thromboinflammation, arteriolar injury, arterial injury, deep vein thrombosis (DVT), venous thromboembolism (VTE), pulmonary embolism, thrombophilia, peripheral artery disease, ischemic heart disease, porto-mesenteric vein thrombosis, Budd-Chiari syndrome, catheter-associated thrombosis, cerebral vein thrombosis, mesenteric ischemia, and cerebrovascular disease.
